(19) United States
(12) Patent Application Publication
(10) Pub. No.: US 2004/0214238 A1
(43) Pub. Date: Oct. 28, 2004

(54) Nociceptive neuron specific calcium channel isoform and uses thereof

(75) Inventors: Diane Lipscombe, Barrington, RI (US); Andrew J. Castiglioni, Arlington, MA (US); Thomas J. Bell, Turnersville, NJ (US); Christopher J. Thaler, Newtown, PA (US)

Correspondence Address: WOLF GREENFIELD & SACKS, PC, FEDERAL RESERVE PLAZA, 600 ATLANTIC AVENUE, BOSTON, MA, 02210-2211, US

(73) Assignee: Brown University Research Foundation, Providence, RI (US)

(21) Appl. No.: 10/736,883

(22) Filed: Dec. 15, 2003

Related U.S. Application Data
(60) Provisional application No. 60/443,474, filed on Jan. 29, 2003.

(51) Int. Cl. G01N 33/53; G01N 33/567; C12N 5/08; C07K 14/705

(52) U.S. Cl. 435/7.2; 435/368; 530/350

(57) ABSTRACT

The invention pertains to N-type calcium channel isoforms containing exon 37a (Cav2.2e[37a]), which are specifically expressed in nociceptive neurons, and nucleic acids relating thereto. Also included are methods for identifying antagonists of Cav2.2e[37a] subunit activity that act by inhibiting the expression or function of the subunit. The invention further relates to methods of using such nucleic acids, polypeptides, and inhibitor molecules in the treatment and/or diagnosis of disease, such as in methods for treating pain, particularly neuropathic pain.

### RELATED APPLICATIONS

This application claims the benefit under 35 U.S.C. § 19(e) of U.S. provisional application serial No. 60/443,474, filed Jan. 29, 2003, the disclosure of which is incorporated by reference herein.

### GOVERNMENT SUPPORT

This work was funded in part by the National Institutes of Health under grant numbers NS29967 and NS43082. The government may have certain rights in this invention.

### FIELD OF THE INVENTION

The invention pertains to N-type calcium channel subunit isoforms that are preferentially expressed in nociceptive neurons.

### BACKGROUND OF THE INVENTION

Nociceptive neurons in the dorsal root ganglia (DRG) respond to a wide variety of stimuli including heat, protons, and capsaicin (McCleskey and Gold, 1999). Nociceptive neurons have been identified based on a number of criteria including morphological, biophysical, and molecular properties. Capsaicin-responsiveness has been used as an important functional indicator of a sub-set of nociceptors that are also heat sensitive (Caterina and Julius, 2001; McCleskey and Gold, 1999).

Voltage gated calcium channels, also known as voltage dependent calcium channels, are multisubunit membrane spanning proteins which permit controlled calcium influx from an extracellular environment into the interior of a cell. Several types of voltage gated calcium channel have been described in different tissues, including N-type, P/Q-type, L-type and T-type channels. A voltage gated calcium channel permits entry into the cell of calcium upon depolarization of the membrane of the cell, which is a lessening of the difference in electrical potential between the outside and the inside of the cell.

Voltage-gated calcium (Ca) channels expressed in nociceptive neurons are present at presynaptic nerve terminals in the dorsal horn of the spinal cord where they regulate transmitter release. Consistent with this, the selective inhibitor of the N-type calcium channel, MVIIA which is also called SNX 111 or ziconotide, is a powerful analgesic (Bowersox et al., 1996; Brose et al., 1997; Chaplan et al., 1994; Cox, 2000). Further, mice lacking the N-type Ca_{V}2.2 subunit have higher pain thresholds compared to wild type (Hatakeyama et al., 2001; Kim et al., 2001; Saegusa et al., 2001; Saegusa et al., 2002). The N-type channel is also present at numerous other synapses and its importance in maintaining sympathetic tone is a major complication in analgesic therapy with N-type channel blockers (Ino et al., 2001; Miljanich and Ramachandran, 1995; Vanegas and Schaible, 2000). Consequently, there is considerable interest in establishing whether nociceptive neurons express a unique class of N-type channel. This possibility has gained momentum with growing appreciation that the Ca_{V}2.2 gene, which encodes the functional core of the N-type Ca channel, is subject to extensive alternative splicing that in some cases is tissue-specific (Lipscombe et al., 2002).

Previous studies have compared calcium currents in nociceptive and non-nociceptive neurons distinguished on the basis of various criteria (Blair and Bean, 2002; Cardenas et al., 1995; Petruska et al., 2000; Regan et al., 1991; Scroggs and Fox, 1992a). The most consistent difference reported is the presence of a larger low threshold T-type calcium current in normociceptive compared to nociceptive neurons. Recent studies suggest that the Ca_{V}3.2 T-type channel localizes to mechanoreceptors (Shin et al., 2003). Differences in high threshold, N-type currents of nociceptive compared to non-nociceptive neurons have not been reported.

Molecular analyses of RNA isolated from dorsal root ganglia have, nonetheless, provided evidence for the presence of multiple splice forms of the N-type Ca_{V}2.2 subunit that differ in their intracellular domains, and S3-S4 extracellular linkers (Lin et al., 1997; Lin et al., 1999; Lu and Dunlap, 1999; Pan and Lipscombe, 2000). None of the splice forms of Ca_{V}2.2 identified to date are exclusively expressed in dorsal root ganglia (Lipscombe et al., 2002).

Therefore, there is a need for the identification of splice isoforms of N-type calcium channel subunits that are preferentially or specifically expressed in nociceptive neurons. Identification of such differential expression of isoforms would permit the development of therapeutics which are specific for the distinct isoforms, thereby lessening side effects resulting from the use of therapeutics which are effective for more than one calcium channel isoform.

### SUMMARY OF THE INVENTION

Based upon RT-PCR expression analysis, functional screening for capsaicin-responsiveness, and biophysical analysis at the single cell level, we present herein the identification of a novel splice isoform of the Ca_{V}2.2α subunit of N-type calcium channels, which is preferentially expressed in a sub-set of nociceptive neurons, and which contains exon 37a. The subunit is referred to herein as “Ca_{V}2.2e[37a]”. The presence of exon 37a in Ca_{V}2.2 correlates with significantly larger N-type currents in neurons and non-neuronal expression systems.

We report the preferential expression of a splice variant of the Ca_{V}2.2 calcium channel in neurons sensitive to capsaicin. Given that distribution, it is clear that a screening assay for compounds that would preferentially block pain receptors can be established by comparing the compounds' activity on the Ca_{V}2.2 exon 37a variant and the Ca_{V}2.2 exon 37b variant, the latter being the control. Most ideally, the screening would be carried out in vitro on neurons genetically engineered to express either the 37a or 37b variant and acrivity of the compounds would be measured as a reduction in calcium current.

The invention provides isolated nucleic acid molecules that encode Ca_{V}2.2e[37a], fragments of those molecules, expression vectors containing the foregoing, and isolated host cells transfected with those molecules. The invention also provides isolated Ca_{V}2.2e[37a] polypeptides and fragments thereof, as well as inhibitors of the foregoing nucleic acids and polypeptides which modulate voltage-gated calcium influx. The foregoing can be used in the diagnosis or treatment of conditions characterized by increased or decreased N-type calcium channel activity correlated with the presence of the Ca_{V}2.2e[37a] subunit and can be used in methods to identify molecules that are therapeutically useful to modulate N-type calcium activity correlated with the presence of the Ca_{V}2.2e[37a] subunit, most particularly for treatment of pain (e.g., neuropathic pain), by modulating voltage regulated calcium influx.

The invention involves in one aspect recombinantly expressed Ca_{V}2.2 nucleic acids and polypeptides which include exon 37a or the amino acid sequence encoded thereby (CCRIHYKDMYSLLRCIAPPVGLGKNCPRRLAYK, SEQ ID NO:45). The first amino acid (Cys) in the amino acid sequence set forth as SEQ ID NO:45 is partly encoded by the end of exon 36 and partly encoded by beginning of exon 37a, as the reading frame is not aligned to the exon-exon junctions. The amino acid sequence set forth as SEQ ID NO:45 is identical in human, mouse and rat. The nucleotide sequence that encodes SEQ ID NO:45 is identical in human and rat, but not in mouse (which therefore has a degenerate nucleotide sequence).

In one embodiment, the polypeptide comprises the amino acid sequence of SEQ ID NO:45, and preferably consists of the amino acid sequence of SEQ ID NO:45. In another embodiment the Ca_{V}2.2 polypeptide is a fragment or variant of the foregoing polypeptides, wherein the fragment or variant includes additions, deletions or substitutions of the amino acid sequence of SEQ ID NO:45 which confer the same function as SEQ ID NO:45. Preferred variants include those having additions, substitutions or deletions relative to the Ca_{V}2.2e[37a] polypeptide sequence disclosed herein, particularly those variants which retain one or more of the activities of the Ca_{V}2.2e[37a], including subunits with various other splice variations.

According to another aspect of the invention, isolated cells that recombinantly express an N-type calcium channel comprising a Ca_{V}2.2 subunit that comprises exon e37a (Ca_{V}2.2e[37a]) are provided. In some embodiments, the Ca_{V}2.2e[37a] subunit has a human sequence, a mouse sequence or a rat sequence. In preferred embodiments, the cell is a neuron or an oocyte.

According to still another aspect of the invention, isolated neurons that express an N-type calcium channel comprising a Ca_{V}2.2 subunit that comprises exon e37a (Ca_{V}2.2e[37a]) are provided. In some embodiments, the neuron further expresses a marker of nociceptive neurons. Preferably the marker of nociceptive neurons is Na_{V}1.8 or vanilloid receptor VR1. In particularly preferred embodiments, the neuron expresses both Na_{V}1.8 and vanilloid receptor VR1.

In another aspect of the invention, methods are provided for identifying lead compounds for a pharmacological agent useful in the treatment of disease associated with increased or decreased voltage regulated calcium influx mediated by a N-type calcium channel containing a Ca_{V}2.2e[37a] subunit. The methods include providing a cell or other membrane-encapsulated space comprising a Ca_{V}2.2e[37a] polypeptide; contacting the cell or other membrane-encapsulated space with a candidate pharmacological agent under conditions which, in the absence of the candidate pharmacological agent, cause a first amount of voltage regulated calcium influx into the cell or other membrane-encapsulated space; and determining a test amount of voltage regulated calcium influx as a measure of the effect of the lead compounds for a pharmacological agent on the voltage regulated calcium influx mediated by a N-type calcium channel containing a Ca_{V}2.2e[37a] subunit. A test amount of voltage regulated calcium influx which is less than the first amount indicates that the candidate pharmacological agent is a lead compound for a pharmacological agent which reduces voltage regulated calcium influx. A test amount of voltage regulated calcium influx which is greater than the first amount indicates that the candidate pharmacological agent is a lead compound for a pharmacological agent which increases voltage regulated calcium influx. In some embodiments, the methods also include a step of loading the cell or other membrane-encapsulated space with a calcium-sensitive compound which is detectable in the presence of calcium, wherein the calcium-sensitive compound is detected as a measure of the voltage regulated calcium influx.

In a preferred embodiment, the pharmacological agent that specifically reduces voltage regulated calcium influx mediated by a N-type calcium channel containing a Ca_{V}2.2e[37a] subunit is an agent that reduces N-type calcium channel current densities in nociceptive neurons. In another preferred embodiment, the pharmacological agent that specifically reduces voltage regulated calcium influx mediated by a N-type calcium channel containing a Ca_{V}2.2e[37a] subunit is useful as an analgesic agent.

Methods for identifying compounds which selectively or preferentially bind a N-type calcium channel containing a Ca_{V}2.2e[37a] subunit also are provided in another aspect of the invention. The methods include providing a first cell or membrane encapsulated space which expresses a N-type calcium channel that contains a Ca_{V}2.2e[37a] subunit, providing a second cell or membrane encapsulated space which expresses a N-type calcium channel that does not contain a Ca_{V}2.2e[37a] subunit, wherein the second cell or membrane encapsulated space is identical to the first cell except for the N-type calcium channel expressed, contacting the first cell or membrane encapsulated space and the second cell or membrane encapsulated space with a compound, and determining the binding of the compound to the first cell or membrane encapsulated space and the second cell or membrane encapsulated space. A compound which binds the first cell or membrane encapsulated space but does not bind the second cell or membrane encapsulated space is a compound which selectively binds the N-type calcium channel that contains a Ca_{V}2.2e[37a] subunit. A compound which binds the first cell or membrane encapsulated space in an amount greater than the compound binds the second cell or membrane encapsulated space is a compound which preferentially binds the N-type calcium channel that contains a Ca_{V}2.2e[37a] subunit. In a preferred embodiment, the N-type calcium channel that does not contain a Ca_{V}2.2e[37a] subunit is a N-type calcium channel that contains a Ca_{V}2.2e[37b] subunit.

In a further aspect of the invention, methods for identifying compounds which selectively or preferentially bind to a Ca_{V}2.2e[37a] isoform are provided. The methods include providing a Ca_{V}2.2e[37a] isoform polypeptide or nucleic acid, providing a Ca_{V}2.2e[37b] isoform polypeptide or nucleic acid, contacting the Ca_{V}2.2e[37a] isoform polypeptide or nucleic acid and the Ca_{V}2.2e[37b] subunit isoform polypeptide or nucleic acid with a compound, and determining the binding of the compound to the Ca_{V}2.2e[37a] isoform polypeptide or nucleic acid and the Ca_{V}2.2e[37b] isoform polypeptide or nucleic acid. A compound which binds the Ca_{V}2.2e[37a] isoform polypeptide or nucleic acid but does not bind the human N-type calcium channel Ca_{V}2.2e[37b] isoform polypeptide or nucleic acid is a compound which selectively binds the Ca_{V}2.2e[37a] isoform, and wherein a compound which binds the Ca_{V}2.2e[37a] isoform polypeptide or nucleic acid in an amount greater than the compound binds the Ca_{V}2.2e[37b] isoform polypeptide or nucleic acid is a compound which preferentially binds the Ca_{V}2.2e[37a] isoform.

In the foregoing methods for identifying compounds which selectively or preferentially bind to a Ca_{V}2.2e[37a] isoform, the compound preferably is an antibody or a antigen-binding fragment thereof, a nucleic acid molecule or one or more compounds from a library of molecules. Preferably the library is a natural product library or a library generated by combinatorial chemistry.

According to yet another aspect of the invention, methods for preparing an analgesic agent are provided. The methods include identifying an agent that selectively or preferentially reduces calcium channel current densities in nociceptive neurons mediated by N-type calcium channels containing a Ca_{V}2.2e[37a] subunit, and formulating the agent for administration to a subject in need of such treatment. The methods also can include identifying a compound according to the foregoing methods, and formulating the compound for administration to a subject in need of such treatment.

In another aspect, the invention provides double stranded RNA molecules specific for Ca_{V}2.2e[37a] RNA. Preferably the double stranded RNA molecule is a siRNA molecule useful in RNA interference. Preferred embodiments include double stranded RNA molecules that are 21-23 nucleotides in length and/or that have a 3′ overhang. Preferably the 3′ overhang is 2 nucleotides in length. In still other embodiments, the double stranded RNA molecule is a single molecule that comprises a hairpin structure.

In a further aspect of the invention, methods for inhibiting calcium influx in a neuronal cell mediated by a N-type calcium channel containing a Ca_{V}2.2e[37a] subunit are provided. The methods include contacting the neuronal cell with an amount of a Ca_{V}2.2e[37a] inhibitor effective to inhibit calcium influx in the mammalian cell. In some embodiments, the inhibitor is selected from the group consisting of an antibody which selectively binds the Ca_{V}2.2e[37a] polypeptide, an antisense nucleic acid that reduces expression of a Ca_{V}2.2e[37a] polypeptide, a siRNA that reduces expression of a Ca_{V}2.2e[37a] polypeptide.

According to another aspect of the invention, methods for treating a subject afflicted by pain mediated by a N-type calcium channel containing a Ca_{V}2.2e[37a] subunit are provided. The methods include administering to a subject in need of such treatment an inhibitor of the Ca_{V}2.2e[37a] polypeptide in an amount effective to inhibit voltage regulated calcium influx and thereby to reduce the pain. In some embodiments, the inhibitor is selected from the group consisting of an antibody which selectively binds the Ca_{V}2.2e[37a] polypeptide, an antisense nucleic acid that reduces expression of a Ca_{V}2.2e[37a] polypeptide, a siRNA that reduces expression of a Ca_{V}2.2e[37a] polypeptide.

In the foregoing methods, the inhibitor is administered prophylactically to a subject at risk of being afflicted with pain. Preferably the pain is neuropathic pain.

Use of the foregoing compositions in the preparation of a medicament, and particularly in the preparation of a medicament for the treatment of stroke, pain (e.g., neuropathic pain), traumatic brain injury, or a condition which results from excessive or insufficient voltage regulated calcium influx, is provided.

These and other aspects of the invention are described in greater detail below.

### BRIEF DESCRIPTION OF THE FIGURES

FIG. 1 shows RT-PCR analysis of e37a and e37b splice variants.

FIG. 2 shows RT-PCR analysis of e37a and e37b in single DRG neurons.

FIG. 3 shows single cell RT-PCR (scRT-PCR) analysis of Ca_{V}2.2 e37a and e37b in capsaicin-responsive and capsaicin-non-responsive neurons.

FIG. 4 shows the experimental protocol used in various experiments.

FIG. 5 shows whole cell calcium currents in capsaicin-responsive and capsaicin-non-responsive neurons.

FIG. 6 shows that ω-Ctx GVIA-sensitive calcium currents in capsaicin-responsive and capsaicin-non-responsive neurons.

FIG. 7 shows that LVA currents rundown significantly over a 5 minute time period in capsaicin-non-responsive neurons.

FIG. 8 shows ω-Ctx GVIA-sensitive calcium currents in capsaicin-responsive neurons that contain and lack e37a.

FIG. 9 shows competitive RT-PCR analysis of e37a and e37b in whole tissue and single neurons.

FIG. 10 shows that multiple splice forms of Ca_{V}2.2 are expressed in dorsal root ganglia. FIG. 10a, Putative membrane topology of the Ca_{V}2.2 subunit. The approximate location of constitutively expressed exons (horizontal black lines) and alternatively spliced exons, e18a, e24a, e31a and e37a/e37b (blue circles) are shown. FIG. 10b, RT-PCR analysis of e18a, e24a, and e31a in mRNA isolated from rat DRG. Primers flanked each splice site and generated the following products: 227 and 290 bp for Δe18a and +e18a; 114 and 126 bp for Δe24a and +e24a; and 169 and 175 bp for Δe31 a and +e31 a. PCR-derived cDNA products were separated on a 2% agarose (e18a) or 4% Metaphor agarose gel (e24a and e31a). Results are consistent with previous analyses of these sites of alternative splicing by RT-PCR and ribonuclease protection assays (Lin et al., 1997; Lin et al., 1999; Pan and Lipscombe, 2000).

FIG. 11 shows that capsaicin-responsiveness in DRG neurons is correlated with the presence of VR1. DRG neurons were screened for capsaicin-responsiveness by whole cell recording (n=269 cells). Whole cell currents recorded from FIG. 11a, a non-responsive neuron and FIG. 11b, a capsaicin-responsive neuron. The membrane potential was voltageclamped at −60 mV. The horizontal bar indicates the time and duration of capsaicin application (2 μM). No inward current was detected in 141 neurons. Inward currents were induced in 128 neurons during capsaicin challenge, with an average amplitude of 986±118 pA. FIG. 1c, PCR-derived cDNA products amplified in two sets of reactions from 5 individual neurons (lanes 1-5) using VR1 and GAPDH-specific primers. The predicted size of PCR products was 125 bp and 274 bp, respectively. The capsaicin-responsiveness of each cell is indicated between gels (+ or −). FIG. 1d, Histogram showing the percentage of non-responsive cells (gray) and capsaicin-responsive cells (red) containing VR1. PCR products were amplified in 89% of capsaicin-responsive cells (25 of 28) with VR1 primers compared to 13% of non-responsive cells (2 of 15).

FIG. 12 shows that expression patterns of exons, e18a, e24a, and e31a, do not correlate with capsaicin-responsiveness. Representative gels showing single cell RT-PCR-derived cDNA products amplified using Ca_{V}2.2-specific primers flanking exons FIG. 12a, eb18a; FIG. 3b, e24a; and FIG. 12c, e31a, together with histograms summarizing the distribution of exons based on capsaicin-responsiveness. Control GAPDH-specific primers are used in each single cell reaction. Products amplified from four cells are shown for each primer pair (lanes 1-4). In FIG. 12c, the first two lanes show products amplified from Ca_{V}2.2e[Δ31a] and Ca_{V}2.2e[+e31 a] clones to establish that a 6 bp difference is resolvable in a 4% Metaphor gel. Sizes of cDNA products were respectively, 227 bp and 290 bp for Δe18 and +e18a; 114 bp and 126 for Δe24a and +e24a; and 169 bp and 175 bp for .e31a and +e31a. Histograms show percent cells that lack the specified exon (A) and that express both splice isoforms lacking and containing the exon (both). Histograms separate cells based on capsaicin-non-responsiveness (gray) and capsaicin-responsiveness (red). The total number of cells analyzed is shown below each histogram. Capsaicin responsiveness of each cell is indicated between gels (+ or −).

FIG. 13 shows that exon 37a is expressed exclusively in dorsal root ganglia. FIG. 13a, Splicing pattern of mutually exclusive exons e37b and e37a of Ca_{V}2.2e[37a] based on analysis of the public rat genomic sequence (accession number NW_{—}043710) and our sequencing (accession number AY211499). Exons are denoted with solid bars and introns with horizontal lines. Exon lengths are 128, 97, 97, and 109 bps for e36, e37a, e37b, and e38 respectively (accession numbers AY211499 and AY211500). 37a amino acid sequence is CCR1 YKDMYSLLRCIAPPVGLGKNCPRRLAY (SEQ ID NO:46); 37b amino acid is sequence CGRISYNDMFEMLKHMSPPLGLGKKCPARVAY (SEQ ID NO:47) FIG. 13b, Expression pattern of e37b and e37a in RNA isolated from various regions of the adult rat nervous system. SCG, superior cervical ganglia; DRG, dorsal root ganglia; SC, spinal cord; MD, medulla; MB, midbrain; CM, cerebellum; TH, thalamus; HC, hippocampus; CX, cortex. Primers were exon-specific for e37a and e37b. PCR-derived products were separated on a 3% agarose gel. Each lane contains equal amounts of PCR reaction. FIG. 13c and FIG. 13d, Levels of Ca_{V}2.2 mRNA containing e37a and e37b were estimated in P5 (FIG. 13c), and adult (FIG. 13d) DRG tissue by competitive RT-PCR. Each primer pair generated two PCR products, 108 bp from Ca_{V}2.2 cDNA and 135 bp from competitive template. Gel shows products amplified by RT-PCR of RNA isolated from whole DRG (500 pg per reaction=˜5 single cells) for e37a and e37b in the presence of serial dilutions of competitive template (10⁻¹⁸ to 10⁻²² M). In P5 tissue, FIG. 13c, the e37b competitive template product was completely depleted at 5×10⁻²¹ M by the tissue-derived e37b template. The two were approximately equal in intensity at 5×10⁻²⁰ M. The e37a competitive template product was completely depleted at 5×10⁻²² M by the tissue-derived e37a template. The two were approximately equal in intensity at 5×10⁻²¹ M. In adult tissue, FIG. 13d, the e37b competitive template product was completely depleted at 1×10⁻²¹ M by the tissue-derived e37b template. The two were approximately equal in intensity at 5×10⁻²⁰ M. The e37a competitive template product was completely depleted at 1×10⁻²² M by the tissue-derived e37a template. The two were approximately equal in intensity at 5×10⁻²¹ M. These gels are representative of three experiments that gave similar results.

FIG. 14 shows that exon 37a is preferentially expressed in nociceptive neurons. Single neurons were analyzed by RT-PCR and the expression pattern of e37a correlated with capsaicin-responsiveness. FIG. 14a and FIG. 14b, Histogram summary showing the number of cells expressing e37b and e37a in capsaicin-non-responsive neurons (gray) and responsive neurons (red). e37a-specific primers amplified products in 32 of 58 capsaicin-responsive and 5 of 27 non-responsive neurons. FIG. 14c, Histogram summary of the number of cells expressing e37a, Na_{V}1.8, and both e37a and Na_{V}1.8, in 24 capsaicin-responsive cells. FIG. 14d, Representative gels showing RT-PCR products amplified with e37a, e37b and GAPDH-specific primers from four single cells (lanes 1-4). The capsaicin-responsiveness of each cell is indicated between gels (+ or −). FIG. 14e, Gels showing RT-PCR products amplified with Na_{V}1.8, e37a, and GAPDH-specific primers from four neurons (lanes 1-4). The capsaicin-responsiveness of each neuron is indicated between gels (+ or −).

FIG. 15 depicts a comparison of calcium channel currents in capsaicin-non-responsive and responsive neurons. FIG. 15a, Average, peak current-voltage relationships for whole cell calcium currents measured in capsaicin-responsive (●) and non-responsive (∘) neurons of dorsal root ganglia. Average, peak current density and capacitance were, for capsaicin-responsive neurons: 135±19 pA/pF and 18±2 pF, n=20; for capsaicin-non-responsive neurons: 123±17 pA/pF and 27±3 pF, n=9. Curves are fit with the sum of two Boltzmann-GHK functions. Estimated V_{1/2} values were ˜−45 mV and ˜−15 mV for low and high voltage-activated currents, respectively. Upper inset: Representative, low voltage-activated and high voltage-activated whole cell calcium currents activated by voltage steps to −40 mV and −5 mV, respectively, from a holding potential of −80 mV from a capsaicin-non-responsive neuron. Lower inset: Same as upper inset from a capsaicin-responsive neuron. Scale bars: 1 nA, 10 ms. FIG. 15b, Average, peak current voltage relationships for ω-Ctx GVIA-subtracted calcium current in capsaicin-responsive (●) and non-responsive (∘) neurons. Average, peak current densities were 111±12 pA/pF (n=20) for capsaicin-responsive compared to 72±8 pA/pF (n=9) for non-responsive neurons. These values are significantly different (p<0.05). The ω-Ctx GVIA-sensitive current was 71±2% of the total whole cell calcium current in capsaicin-responsive neurons and 68±2% of whole cell current in non-responsive neurons. Curves are fit with the sum of two Boltzmann-GHK functions. Average V_{1/2} and k values were calculated from fits of individual N-type current-voltage relationships. In capsaicin-non-responsive cells, for the low voltage-activated componefit, V_{1/2} and k values were −25±4 mV and 4.8±0.5 compared to −21+2 mV and 6±0.6 for capsaicin-responsive cells. In capsaicin non-responsive neurons average V_{1/2} and k values were, for the high voltage-activated component: −16+2 mV and 5.4±0.6 compared to −15±1 mV and 5.2±0.3 for capsaicin-responsive cells. Values of V_{1/2} and k were not significantly different between capsaicin-responsive and capsaicin-non-responsive neurons (p>0.05). Inset, Representative ω-Ctx GVIA-sensitive current recorded at −5 mV from a capsaicin-responsive neuron (lower trace) and non-responsive neuron (upper trace). Scale bar: 25 pA/pF, 10 ms. Data are mean±se.

FIG. 16 show that exon 37a expression is associated with larger N-type currents in capsaicin-responsive neurons. FIG. 16a, Average, peak current-voltage relationships of ω-Ctx GVIA-sensitive calcium current in capsaicin-responsive neurons that contain (●) and lack (∘) e37a. Average peak current density at 0 mV and capacitance of responsive neurons that contain e37a were 122±11 pA/pF and 20±3 pF (n=8) compared to 76±3 pA/pF and 18±1 pF for neurons that lack e37a (n=8). Peak current densities are significantly greater in neurons containing e37a (p<0.05). Current densities were significantly different between splice isoforms when compared at −10 mV, −5 mV, 0 mV, +5 mV, and +10 mV (p<0.05). Curves are Boltzmann-linear IV fits. Average V_{1/2} and k values are −12.7±1.8 mV and 4.6±0.4, n=8, for neurons containing e37a compared to −13.6±1.7 mV and 5.4±0.3, n=8, for neurons lacking e37a. V_{1/2} and k values are not significantly different between the two groups (p>0.05). Inset shows examples of toxin-subtracted currents from neurons containing (●) and lacking (∘) exon 37a. Scale bars are 10 ms and 20 pA/pF. FIG. 16b, Averages of time constants estimated from fits of the activation phase of toxin-subtracted N-type currents induced by step depolarizations to indicated test potentials, from capsaicin-responsive neurons containing (●) and lacking (∘) exon 37a. FIG. 16c, Average time constants estimated from fits of the inactivation kinetics of toxin-subtracted N-type currents induced by step depolarizations to indicated test potentials, from capsaicin-responsive neurons containing (●) and lacking (∘) exon 37a. FIG. 16d, Representative gels showing RT-PCR products amplified from four single cells (lanes 1-4) with primers specific for e37a, e37b, and GAPDH. Cells were used in the analysis shown in FIG. 16a. Data are mean±se.

FIG. 17 shows that Ca_{V}2.2e[37a] clones induce N-type currents in Xenopus oocytes that are significantly larger compared to Ca_{V}2.2e[37b]. FIG. 17a, Average peak current-voltage relationships in oocytes expressing Ca_{V}2.2e[37a] ( ) and Ca_{V}2.2e[37b] (∘). After 5 days post injection, average Ca_{V}2.2e[37a] peak currents were 211±2 nA (n=8) compared to 134+4 nA for Ca_{V}2.2e[37b] (n=8). Peak Ca_{V}2.2e[37a] currents were significantly greater than Ca_{V}2.2e[37b] at day 4, 5 and 6 after injection (p<0.05). The dotted line shows the predicted current voltage-relationship of Ca_{V}2.2e[37b] calculated using the Boltzmann activation curve of Ca_{V}2.2e[37a] shown in FIG. 17b. This predicted curve demonstrates that an 8 mV left shift in voltage-dependence of channel activation (see FIG. 17b) is insufficient to account for the significantly larger currents of Ca_{V}2.2e[37a] compared to Ca_{V}2.2e[37b]. Inset: Representative Ca_{V}2.2e[37a] and Ca_{V}2.2e[37b] currents induced by step depolarizations to peak current (−5 mV for Ca_{V}2.2e[37a] and 0 mV for Ca_{V}2.2e[37b]) from a holding potential of −80 mV. Scale bar: 50 nA, 20 ms. V_{1/2} and k values were estimated from Boltzmann-GHK fits to individual data sets. Average V_{1/2} values are −17.9±0.6 mV, n=8, for Ca_{V}2.2e[37a] and −9.7±0.4 mV, n=8, for Ca_{V}2.2e[37b]. k values are 5.3±0.1 for Ca_{V}2.2e[37a] and 5.1±0.1 for Ca_{V}2.2e[37b]. Average, macroscopic activation time constants Tact are 7.2±0.5 ms for Ca_{V}2.2e[37a], n=8, and 10.6±0.5 ms for Ca_{V}2.2 e[37b], n=9. These values are significantly different (p<0.05). Peak currents in oocytes expressing Ca_{V}2.2e[37a] were 186±2 nA (n=4), 211±2 nA (n=8), and 387±20 nA (n=8) at days 4, 5 and 6 days post injection, respectively. Compared to 68±2 nA (n=3), 134±2 nA (n=8), and 204±10 nA (n=8) at 4, 5 and 6 days post injection, respectively, in oocytes expressing Ca_{V}2.2e[37b]. In all cases values between splice isoforms were significantly different on a given day (p<0.05). FIG. 17b, Normalized, averaged activation curves for N-type currents in oocytes expressing Ca_{V}2.2e[37a] (●) and Ca_{V}2.2e[37b] (∘). Curves were generated from slope conductances calculated from peak current-voltage relationships shown in FIG. 8a, and assuming a reversal potential of +40 mV. Boltzmann functions were fit to individual curves and used to calculate average values for V_{1/2} and k. These were for Ca_{V}2.2e[37a]: −19.7±0.6 mV and 4.4±0.2; and for Ca_{V}2.2e[37b]: −11.7±0.5 mV and 4.7±0.1. V_{1/2} values are significantly different (p<0.05); k values are not significantly different. FIG. 17c, Normalized, averaged steady-state inactivation curves for N-type currents in oocytes expressing Ca_{V}2.2e[37a] (●) and Ca_{V}2.2e[37b] (∘). Curves were generated from peak currents elicited by 300 ms test pulses to −5 mV (Ca_{V}2.2e[37a], n=12) or 0 mV (Ca_{V}2.2e[37b], n=11) after 20 second conditioning prepulses to voltages ranging from −100 mV to +20 mV. Barium (5 mM) was the charged carrier. Peak currents are plotted as a fraction of the maximum current at the indicated holding potentials. V_{1/2} and k values were estimated from Boltzmann fits to data from individual cells. Average V_{1/2} and k values were for Ca_{V}2.2e[37a]: −72.7±0.8 mV and 8.1±0.4; and for Ca_{V}2.2e[37b]: −72.0±0.4 mV 8.1±0.6. Values are not significantly different. Inactivation kinetics were also measured, Ca_{V}2.2e[37a]: τ_{inact-1}=393±17 ms and τ_{inact-2}=89±5 ms compared to 384±8 ms and 82±2 ms for Ca_{V}2.2e[37b]. Values are not significantly different between splice isoforms. These data are representative of four separate injections. Data are mean±se.

### DETAILED DESCRIPTION OF THE INVENTION

The present invention in one aspect involves the discovery that a particular splice isoform of the N-type calcium channel Ca_{V}2.2α (gene CACNA1B) containing the amino acid sequence encoded by exon 37a, referred to herein as the Ca_{V}2.2e[37a] isoform, is preferentially and specifically expressed in nociceptive neurons. The invention also pertains to the use of these isoforms in drug discovery to identify agents that selectively inhibit the function of N-type calcium channels containing a Ca_{V}2.2e[37a] subunit, particularly in cell-based assays, and agents identified by such methods. The invention also includes agents that modulate expression of the Ca_{V}2.2e[37a] gene transcript (transcription or translation), such as siRNA that selectively reduces expression of Ca_{V}2.2e[37a], and it use in treating conditions that involve this subunit, particularly pain.

As used herein, Ca_{V}2.2e[37a] refers to any N-type calcium channel Ca_{V}2.2 subunit that contains an amino acid sequence encoded by exon 37a, or nucleic acid molecules encoding such subunits. The human, mouse and rat Ca_{V}2.2e exon 37a-encoded amino acid sequence is presented as SEQ ID NO:45; the equivalent exon in other species can be identified by routine experimental procedures including homology analysis of a species' genomic sequence, transcript sequences (e.g., mRNA), etc. as will be understood by those of skill in the art. N-type calcium channel Ca_{V}2.2 subunits that do not contain exon 37a have been deposited in GenBank under the following accession numbers.

TABLE 1
Species Homologs

| | Accession | SEQ ID |
|---|---|---|
| Species | number | NO |
| Rattus norvegicus | AF055477 | 27, 28 |
| | M92905 | 29, 30 |
| Homo sapiens | M94172 | 31, 32 |
| | M94173 | 33, 34 |
| Mus musculus | AF042317 | 37, 38 |
| | U04999 | 39, 40 |
| Oryctolagus cuniculus (rabbit) | D14157 | 41, 42 |
| Bos taurus | AF173882 | 43, 44 |

The human nucleotide sequence of exon 37a (then called exon 42) was identified and deposited under accession number AF238295; it is provided here as SEQ ID NO:35. The amino acid sequence encoded by this sequence is provided here as SEQ ID NO:36. The amino acid sequence (YDPAACCRIHYKDMYSLLRCIAPPVGLGKNCPRRLAYK; SEQ ID NO:36) includes amino acids (double underline) encoded by the 3′ end of exon 41 (nucleotides 1-17) and amino acids (underlined) encoded by exon 42 (nucleotides 4510-4606). Note that amino acid 6 (Cys) is partly encoded by both exon nucleotide sequences.

Because the present Ca_{V}2.2e[37a] isoform is a splice variant of the N-type calcium channel Ca_{V}2.2 subunit, it is apparent that the invention is meant to embrace N-type calcium channel Ca_{V}2.2 subunit variants which vary by alternative splicing of sequences other than the exon 37a insert (SEQ ID NO:45), and nucleotide sequence encoding such polypeptides. For example, the invention embraces the splice variant polypeptides, and nucleic acid molecules encoding such splice variant polypeptides, as described in U.S. Pat. No. 6,353,091. As shown in the Examples, the Ca_{V}2.2e[37a] subunit is significantly and differentially expressed in certain subsets of neurons that exert different function in different parts of the brain. This opens the possibility for the selective treatment of disorders which involve those parts of the brain.

The invention involves in one aspect Ca_{V}2.2e[37a] nucleic acids and polypeptides, as well as therapeutics relating thereto. The invention also embraces isolated functionally equivalent variants, useful analogs and fragments of the foregoing nucleic acids and polypeptides; complements of the foregoing nucleic acids; and molecules which selectively bind the foregoing nucleic acids and polypeptides.

The Ca_{V}2.2e[37a] nucleic acids and polypeptides of the invention are isolated. The term “isolated”, as used herein in reference to a nucleic acid molecule, means a nucleic acid sequence: (i) amplified in vitro by, for example, polymerase chain reaction (PCR); (ii) synthesized by, for example, chemical synthesis; (iii) recombinantly produced by cloning; or (iv) purified, as by cleavage and electrophoretic or chromatographic separation. The term “isolated”, as used herein in reference to a polypeptide, means a polypeptide encoded by an isolated nucleic acid sequence, as well as polypeptides synthesized by, for example, chemical synthetic methods, and polypeptides separated from biological materials, and then purified, using conventional protein analytical or preparatory procedures, to an extent that permits them to be used according to the methods described herein.

As used herein a Ca_{V}2.2e[37a] nucleic acid refers to an isolated nucleic acid molecule which codes for a Ca_{V}2.2 subunit protein that includes exon 37a. Ca_{V}2.2e[37a] nucleic acids are those nucleic acid molecules which code for Ca_{V}2.2e[37a] polypeptides which include the sequence of SEQ ID NO: 45. The Ca_{V}2.2e[37a] nucleic acids of the invention also include alleles and species homologs of the foregoing nucleic acids, as well as fragments of the foregoing nucleic acids, provided that the allele, species isoform or fragment contains exon 37a (i.e., the pertinent exon of the particular species that is homologous to those described herein). Such fragments can be used, for example, as probes in hybridization assays, as primers in a polymerase chain reaction (PCR), and for reconbination with exon 37b containing nucleic acids. In a preferred embodiment, exon 37a-specific fragments and their complements can be used in the preparation of double stranded RNA molecules (such as siRNA molecules and hairpins) useful for inhibition of expression by RNA interference (RNAi). Preferred Ca_{V}2.2e[37a] nucleic acids include nucleotides 4510-4606 of SEQ ID NO:35 (Accession number AF238295). Complements of the foregoing nucleic acids also are embraced by the invention.

As used herein “Ca_{V}2.2e[37a] activity” refers to an ability of a molecule to modulate voltage regulated calcium influx. As shown in the Examples, neurons and other cells that express N-type calcium channels containing Ca_{V}2.2e[37a] have significantly larger N-type currents. Thus, a molecule which inhibits Ca_{V}2.2e[37a] activity (an antagonist) is one which inhibits voltage regulated calcium influx via this calcium channel and a molecule which increases Ca_{V}2.2e[37a] activity (an agonist) is one which increases voltage regulated calcium influx via this calcium channel. Changes in Ca_{V}2.2e[37a] activity can be measured by changes in voltage regulated calcium influx by in vitro assays such as those disclosed herein, including patch-clamp assays, and other assays known to those of skill in the art, such as assays employing calcium sensitive fluorescent compounds such as fura-2.

Alleles of the Ca_{V}2.2e[37a] nucleic acids of the invention can be identified by conventional techniques. For example, alleles of Ca_{V}2.2e[37a] can be isolated by hybridizing a probe which includes nucleotides 4510-4606 of SEQ ID NO:35, or another nucleotide sequence that encodes SEQ ID NO:45 (except the first amino acid, which as noted herein is encoded partly by exon 36 and partly by exon 37a), under stringent conditions with a cDNA library and selecting positive clones. Thus, an aspect of the invention is those nucleic acid sequences which code for Ca_{V}2.2e[37a] polypeptides and which hybridize to a nucleic acid molecule consisting of Ca_{V}2.2 exon 37a under stringent conditions. The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄(pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, the membrane upon which the DNA is transferred is washed at 2×SSC at room temperature and then at 0.1×SSC/0.1×SDS at temperatures up to 65° C.

There are other conditions, reagents, and so forth which can be used, which result in a similar degree of stringency. The skilled artisan will be familiar with such conditions, and thus they are not given here. It will be understood, however, that the skilled artisan will be able to manipulate the conditions in a manner to permit the clear identification of alleles of Ca_{V}2.2e[37a] nucleic acids of the invention. The skilled artisan also is familiar with the methodology for screening cells and libraries for expression of such molecules which then are routinely isolated, followed by isolation of the pertinent nucleic acid molecule and sequencing.

In screening for Ca_{V}2.2e[37a] nucleic acids, nucleic acid amplification techniques such as PCR can be performed as known to one of ordinary skill in the art. In addition, hybridization techniques such as Southern and Northern blots may be performed using the foregoing stringent conditions, together with a labeled (e.g., fluorescently, radioactively) probe. After washing the membrane to which the DNA is finally transferred, the membrane can be placed against various photon capture devices (e.g., phosphorimager) or X-ray film to detect the radioactive signal.

Alleles of Ca_{V}2.2e[37a] nucleic acids also can be identified using in silico techniques of database mining, sequence comparison, etc. as will be known to one of ordinary skill in the art.

The Ca_{V}2.2e[37a] nucleic acids of the invention also include degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating Ca_{V}2.2e[37a] polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides isolated fragments of Ca_{V}2.2e[37a] nucleic acids, which include at least part of the nucleotide sequence that encodes SEQ ID NO:45. The fragments can be used as probes in Southern blot assays to identify such nucleic acids, or can be used in amplification assays such as those employing PCR. Smaller fragments are those comprising 12, 13, 14, 15, 16, 17, 18, 20, 22, 25, 30, 40, 50, or 75 nucleotides, and every integer therebetween and are useful e.g. as primers for nucleic acid amplification procedures. As known to those skilled in the art, larger probes such as 100, 200, 250, 300, 400 or more nucleotides are preferred for certain uses such as Southern blots, while smaller fragments will be preferred for uses such as PCR. Fragments also can be used to produce fusion proteins for generating antibodies or determining binding of the polypeptide fragments. Likewise, fragments can be employed to produce non-fused fragments of the Ca_{V}2.2e[37a] polypeptides, useful, for example, in the preparation of antibodies, in immunoassays, and the like. The foregoing nucleic acid fragments further can be used as antisense molecules or siRNA molecules to inhibit the expression of Ca_{V}2.2e[37a] nucleic acids and polypeptides, particularly for therapeutic purposes as described in greater detail below.

The invention also includes functionally equivalent variants of the Ca_{V}2.2e[37a], which include variant nucleic acids and polypeptides that retain one or more of the functional properties of the Ca_{V}2.2e[37a], such as the different current densities as described in greater detail below. For example, functionally equivalent variants include a Ca_{V}2.2e[37a] molecule which has had a portion of the extracellular domain removed or replaced by a similar domain from another calcium channel α₁ subunit (e.g. a “domain-swapping” variant). Other functionally equivalent variants will be known to one of ordinary skill in the art, as will methods for preparing such variants. The activity of a functionally equivalent variant can be determined using the methods provided, e.g., herein, in Lin et al., Neuron 18:153-166, 1997, and in U.S. Pat. No. 5,429,921. Such variants are useful, inter alia, in assays for identification of compounds which bind and/or regulate the calcium influx function of the Ca_{V}2.2e[37a], and for determining the portions of the Ca_{V}2.2e[37a] which are required for calcium influx activity.

Variants that are non-functional also can be prepared as described above. Such variants are useful, for example, as negative controls in experiments testing subunit activity, and as inhibition of nociceptive-specific N-type calcium channel activity.

A Ca_{V}2.2e[37a] nucleic acid, in one embodiment, is operably linked to a gene expression sequence which directs the expression of the Ca_{V}2.2e[37a] nucleic acid within a eukaryotic or prokaryotic cell. The “gene expression sequence” is any regulatory nucleotide sequence, such as a promoter sequence or promoter-enhancer combination, which facilitates the efficient transcription and translation of the Ca_{V}2.2e[37a] nucleic acid to which it is operably linked. The gene expression sequence may, for example, be a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, β-actin promoter and other constitutive promoters. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney murine leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In general, the gene expression sequence shall include, as necessary, 5′ non-transcribing and 5′ non-translating sequences involved with the initiation of transcription and translation, respectively, such as a TATA box, capping sequence, CAAT sequence, and the like. Especially, such 5′ non-transcribing sequences will include a promoter region which includes a promoter sequence for transcriptional control of the operably joined Ca_{V}2.2e[37a] nucleic acid. The gene expression sequences optionally includes enhancer sequences or upstream activator sequences as desired.

The Ca_{V}2.2e[37a] nucleic acid sequence and the gene expression sequence are said to be “operably linked” when they are covalently linked in such a way as to place the transcription and/or translation of the Ca_{V}2.2e[37a] coding sequence under the influence or control of the gene expression sequence. If it is desired that the Ca_{V}2.2e[37a] sequence be translated into a functional protein, two DNA sequences are said to be operably linked if induction of a promoter in the 5′ gene expression sequence results in the transcription of the Ca_{V}2.2e[37a] sequence and if the nature of the linkage between the two DNA sequences does not (1) result in the introduction of a frame-shift mutation, (2) interfere with the ability of the promoter region to direct the transcription of the Ca_{V}2.2e[37a] sequence, or (3) interfere with the ability of the corresponding RNA transcript to be translated into a protein. Thus, a gene expression sequence would be operably linked to a Ca_{V}2.2e[37a] nucleic acid sequence if the gene expression sequence were capable of effecting transcription of that Ca_{V}2.2e[37a] nucleic acid sequence such that the resulting trariscript might be translated into the desired protein or polypeptide.

The Ca_{V}2.2e[37a] nucleic acid and the Ca_{V}2.2e[37a] polypeptide (including the Ca_{V}2.2e[37a] inhibitors described below) of the invention can be delivered to the eukaryotic or prokaryotic cell alone or in association with a vector. In its broadest sense, a “vector” is any vehicle capable of facilitating: (1) delivery of a Ca_{V}2.2e[37a] nucleic acid or polypeptide to a target cell or (2) uptake of a Ca_{V}2.2e[37a] nucleic acid or polypeptide by a target cell. Preferably, the vectors transport the Ca_{V}2.2e[37a] nucleic acid or polypeptide into the target cell with reduced degradation relative to the extent of degradation that would result in the absence of the vector. Optionally, a “targeting ligand” can be attached to the vector to selectively deliver the vector to a cell which expresses on its surface the cognate receptor (e.g. a receptor, an antigen recognized by an antibody) for the targeting ligand. In this manner, the vector (containing a Ca_{V}2.2e[37a] nucleic acid or a Ca_{V}2.2e[37a] polypeptide) can be selectively delivered to a specific cell. In general, the vectors useful in the invention are divided into two classes: biological vectors and chemical/physical vectors. Biological vectors are more useful for delivery/uptake of Ca_{V}2.2e[37a] nucleic acids to/by a target cell. Chemical/physical vectors are more useful for delivery/uptake of Ca_{V}2.2e[37a] nucleic acids or Ca_{V}2.2e[37a] proteins to/by a target cell.

Biological vectors include, but are not limited to, plasmids, phagemids, viruses, other vehicles derived from viral or bacterial sources that have been manipulated by the insertion or incorporation of the nucleic acid sequences of the invention, and free nucleic acid fragments which can be attached to the nucleic acid sequences of the invention. Viral vectors are a preferred type of biological vector and include, but are not limited to, nucleic acid sequences from the following viruses: retroviruses, such as Moloney murine leukemia virus; Harvey murine sarcoma virus; murine mammary tumor virus; Rous sarcoma virus; adenovirus; adeno-associated virus; SV40-type viruses; polyoma viruses; Epstein-Barr viruses; papilloma viruses; herpes virus; vaccinia virus; and polio virus. One can readily employ other vectors not named but known in the art.

Preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W.H. Freeman C.O., New York (1990) and Murry, E. J. Ed. “Methods in Molecular Biology,” vol. 7, Humana Press, Inc., Clifton, N.J. (1991).

Another preferred virus for certain applications is the adeno-associated virus, a double-stranded DNA virus. The adeno-associated virus can be engineered to be replication-deficient and is capable of infecting a wide range of cell types and species. It further has advantages, such as heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding a Ca_{V}2.2e[37a] polypeptide or fragment or variant thereof. That heterologous DNA (RNA) is placed under operable control of transcriptional elements to permit the expression of the heterologous DNA in the host cell.

Preferred systems for mRNA expression in mammalian cells are those such as the pcDNA series of vectors (available from Invitrogen, Carlsbad, Calif.) that contain a selectable marker such as a gene that confers G418 resistance (which facilitates the selection of stably transfected cell lines) and the human cytomegaloviius (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992).

In addition to the biological vectors, chemical/physical vectors may be used to deliver a Ca_{V}2.2e[37a] nucleic acid or polypeptide to a target cell and facilitate uptake thereby. As used herein, a “chemical/physical vector” refers to a natural or synthetic molecule, other than those derived from bacteriological or viral sources, capable of delivering the isolated Ca_{V}2.2e[37a] nucleic acid or polypeptide to a cell.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vesicles which are useful as a delivery vector in vivo or in vitro. It has been shown that large unilamellar vesicles (LUV), which range in size from 0.2-4.0μ can encapsulate large macromolecules. RNA, DNA, and intact virions can be encapsulated within the aqueous interior and be delivered to cells in a biologically active form (Fraley, et al., Trends Biochem. Sci., v. 6, p. 77 (1981)). In order for a liposome to be an efficient nucleic acid transfer vector, one or more of the following characteristics should be present: (1) encapsulation of the nucleic acid of interest at high efficiency with retention of biological activity; (2) preferential and substantial binding to a target cell in comparison to non-target cells; (3) delivery of the aqueous contents of the vesicle to the target cell cytoplasm at high efficiency; and (4) accurate and effective expression of genetic information.

Liposomes may be targeted to a particular tissue by coupling the liposome to a specific ligand such as a monoclonal antibody, sugar, glycolipid, or protein. Ligands which may be useful for targeting a liposome to a particular cell will depend on the particular cell or tissue type. Additionally when the vector encapsulates a nucleic acid, the vector may be coupled to a nuclear targeting peptide, which will direct the Ca_{V}2.2e[37a] nucleic acid to the nucleus of the host cell.

Liposomes are commercially available from Gibco BRL, for example, as LIPOFECTIN™ and LIPOFECTACE™, which are formed of cationic lipids such as N-[1-(2, 3 dioleyloxy)-propyl]-N,N, N-trimethylammonium chloride (DOTMA) and dimethyl dioctadecylammonium bromide (DDAB). Methods for making liposomes are well known in the art and have been described in many publications. Liposomes also have been reviewed by Gregoriadis, G. in Trends in Biotechnology, V. 3, p. 235-241 (1985).

Other exemplary compositions that can be used to facilitate uptake by a target cell of the Ca_{V}2.2e[37a] nucleic acids include calcium phosphate and other chemical mediators of intracellular transport, microinjection compositions, electroporation and homologous recombination compositions (e.g., for integrating a Ca_{V}2.2e[37a] nucleic acid into a preselected location within a target cell chromosome).

The invention also embraces so-called expression kits, which allow the artisan to prepare a desired expression vector or vectors. Such expression kits include at least separate portions of the previously discussed coding sequences. Other components may be added, as desired, as long as the previously mentioned sequences, which are required, are included.

It will also be recognized that the invention embraces the use of the Ca_{V}2.2e[37a] cDNA sequences in expression vectors, as well as to transfect host cells and cell lines, be these prokaryotic (e.g., E. coli), or eukaryotic (e.g., neurons, oocytes, COS cells, yeast expression systems and recombinant baculovirus expression in insect cells). Especially useful are mammalian cells such as human, pig, goat, primate, mouse, rat, etc., which can be used for the identification of molecules that regulate the function of Ca_{V}2.2e[37a] selectively (e.g., by screening chemical compounds libraries) They may be of a wide variety of tissue types, and include primary cells and cell lines. Specific examples include neuronal cells including PC12 cells, Xenopus oocytes, bone marrow stem cells and embryonic stem cells. The expression vectors require that the pertinent sequence, i.e., those nucleic acids described supra, be operably linked to a promoter.

The invention also provides isolated Ca_{V}2.2e[37a] polypeptides which include the amino acid sequence of SEQ ID NO:45, encoded by the Ca_{V}2.2e[37a] nucleic acids described above. The preferred Ca_{V}2.2e[37a] polypeptide has the amino acid sequence of any of the N-type calcium channel subunit proteins disclosed herein (see e.g., Table 1), with the amino acid sequence of exon 37a (SEQ ID NO:45) substituted for that of exon 37b. Ca_{V}2.2e[37a] polypeptides also embrace alleles, isoforms (such as splice variants), functionally equivalent variants and analogs (those non-allelic polypeptides which vary in amino acid sequence from SEQ ID NO:45 by 1, 2, 3, 4, or 5 amino acids) provided that such polypeptides retain Ca_{V}2.2e[37a] activity. Non-functional variants also are embraced by the invention; these are useful as antagonists of calcium channel function, as negative controls in assays, and the like. Such alleles, isoforms, variants, analogs and fragments are useful, for example, alone or as fusion proteins for a variety of purposes such as to generate antibodies, or as a component of an immunoassay.

Fragments of a polypeptide preferably are those fragments which retain a distinct functional capability of the Ca_{V}2.2e[37a] polypeptide, in particular voltage regulated calcium influx. Other functional capabilities which can be retained in an allele, isoform, variant, analog or fragment (generically called “variant” herein) of a Ca_{V}2.2e[37a] polypeptide include interaction with antibodies and interaction with other polypeptides (such as other subunits of the human N-type calcium channel). Those skilled in the art are well versed in methods for selecting alleles, isoforms, variants, analogs or fragments which retain a functional capability of the Ca_{V}2.2e[37a]. Confirmation of the functional capability of the fragment can be carried out by synthesis of the fragment and testing of the capability according to standard methods. For example, to test the voltage regulated calcium influx of a Ca_{V}2.2e[37a] fragment, one inserts or expresses the fragment in a cell in which calcium influx can be measured. Such methods, which are standard in the art, are described further in the examples.

The invention embraces variants of the Ca_{V}2.2e[37a] polypeptides described above. As used herein, a “variant” of a Ca_{V}2.2e[37a] polypeptide is a polypeptide which contains one or more modifications to the primary amino acid sequence of a Ca_{V}2.2e[37a] polypeptide. Modifications which create a Ca_{V}2.2e[37a] variant can be made to a Ca_{V}2.2e[37a] polypeptide for a variety of reasons, including 1) to reduce or eliminate an activity of a Ca_{V}2.2e[37a] polypeptide, such as voltage gated calcium influx; 2) to enhance a property of a Ca_{V}2.2e[37a] polypeptide, such as protein stability in an expression system or the stability of protein-protein binding; 3) to provide a novel activity or property to a Ca_{V}2.2e[37a] polypeptide, such as addition of an antigenic epitope or addition of a detectable moiety; or 4) to establish that an amino acid substitution, such as a different sequence encoded by an alternatively spliced exon, does or does not affect voltage gated calcium influx. Modifications to a Ca_{V}2.2e[37a] polypeptide are typically made to the nucleic acid which encodes the Ca_{V}2.2e[37a] polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the Ca_{V}2.2e[37a] amino acid sequence, but always including the amino acid sequence encoded by exon 37a.

One of skill in the art will be familiar with methods for predicting the effect on protein conformation of a change in protein sequence, and can thus “design” a variant Ca_{V}2.2e[37a] according to known methods. One example of such a method is described by Dahiyat and Mayo in Science 278:82-87, 1997, whereby proteins can be designed de novo. Additional computer-based modeling methods also are available. The method can be applied to a known protein to vary a only a portion of the polypeptide sequence. By applying the computational methods of Dahiyat and Mayo, specific variants of a Ca_{V}2.2e[37a] polypeptide can be proposed and tested to determine whether the variant retains a desired conformation.

Variants include Ca_{V}2.2e[37a] polypeptides which are modified specifically to alter a feature of the polypeptide unrelated to its physiological activity. For example, cysteine residues can be substituted or deleted to prevent unwanted disulfide linkages. Similarly, certain amino acids can be changed to enhance expression of a Ca_{V}2.2e[37a] polypeptide by eliminating proteolysis by proteases in an expression system (e.g., dibasic amino acid residues in yeast expression systems in which KEX2 protease activity is present).

Mutations of a nucleic acid which encode a Ca_{V}2.2e[37a] polypeptide preferably preserve the amino acid reading frame of the coding sequence, and preferably do not create regions in the nucleic acid which are likely to hybridize to form secondary structures, such as hairpins or loops, which can be deleterious to expression of the variant polypeptide.

Mutations can be made by selecting an amino acid substitution, or by random mutagenesis (e.g., directed molecular evolution, optionally of a selected site) in a nucleic acid which encodes the polypeptide. Variant polypeptides are then expressed and tested for one or more activities to determine which mutation provides a variant polypeptide with a desired property. Further mutations can be made to variants (or to non-variant Ca_{V}2.2e[37a] polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a Ca_{V}2.2e[37a] gene or cDNA clone to enhance expression of the polypeptide.

The activity of variants of Ca_{V}2.2e[37a] polypeptides can be tested by cloning the gene encoding the variant Ca_{V}2.2e[37a] polypeptide into a bacterial or mammalian expression vector, introducing the vector into an appropriate host cell, expressing the variant Ca_{V}2.2e[37a] polypeptide, and testing for a functional capability of the Ca_{V}2.2e[37a] polypeptides as disclosed herein. For example, the variant Ca_{V}2.2e[37a] polypeptide can be tested for ability to provide voltage regulated calcium influx, as set forth below in the examples. Preparation of other variant polypeptides may favor testing of other activities, as will be known to one of ordinary skill in the art.

The skilled artisan will also realize that conservative amino acid substitutions may be made in Ca_{V}2.2e[37a] polypeptides to provide functionally equivalent variants of the foregoing polypeptides, i.e. variants which retain the functional capabilities of the Ca_{V}2.2e[37a] polypeptides. As used herein, a “conservative amino acid substitution” refers to an amino acid substitution which does not alter the relative charge or size characteristics of the polypeptide in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants of the Ca_{V}2.2e[37a] polypeptides include conservative amino acid substitutions of the Ca_{V}2.2 sequences described herein, but excluding the portion of the polypeptide consisting of the amino acid sequence encoded by exon 37a. Other variants can include amino acid substitutions of the exon 37a-encoded sequence, which may be useful for determining which amino acid residues are of importance in the functional activity of Ca_{V}2.2e[37a] protein; such variants can be used in assays to screen molecules for Ca_{V}2.2e[37a] antagonist or agonist activity as well. Exemplary conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Conservative amino-acid substitutions in the amino acid sequence of Ca_{V}2.2e[37a] polypeptide to produce functionally equivalent variants of Ca_{V}2.2e[37a] polypeptides typically are made by alteration of a nucleic acid sequence encoding Ca_{V}2.2e[37a] polypeptides (e.g., nucleotides 5212-5308 of SEQ ID NO:23). Such substitutions can be made by a variety of methods known to one of ordinary skill in the art. For example, amino acid substitutions may be made by PCR-directed mutation, site-directed mutagenesis according to the method of Kunkel (Proc. Nat. Acad. Sci. U.S.A. 82: 488-492, 1985), or by chemical synthesis of a gene encoding a Ca_{V}2.2e[37a] polypeptide. Where amino acid substitutions are made to a small unique fragment of a Ca_{V}2.2e[37a] polypeptide, such as a specific domain, the substitutions can be made by directly synthesizing the peptide. The activity of functionally equivalent variants of Ca_{V}2.2e[37a] polypeptides can be tested by cloning the gene encoding the altered Ca_{V}2.2e[37a] polypeptide into a bacterial or mammalian expression vector, introducing the vector into an appropriate host cell, expressing the altered Ca_{V}2.2e[37a] polypeptide, and testing for the ability of the Ca_{V}2.2e[37a] polypeptide to transduce voltage regulated calcium influx. Peptides which are chemically synthesized can be tested directly for function.

A variety of methodologies well-known to the skilled practitioner can be utilized to obtain isolated Ca_{V}2.2e[37a] molecules. The polypeptide may be purified from cells which naturally produce the polypeptide by chromatographic means or immunological recognition. Alternatively, an expression vector may be introduced into cells to cause production of the polypeptide. In another method, mRNA transcripts may be microinjected or otherwise introduced into cells to cause production of the encoded polypeptide. Translation of mRNA in cell-free extracts such as the reticulocyte lysate system also may be used to produce polypeptide. Those skilled in the art also can readily follow known methods for isolating Ca_{V}2.2e[37a] polypeptides. These include, but are not limited to, immunochromatography, HPLC, size-exclusion chromatography, ion-exchange chromatography and immune-affinity chromatography.

The invention also embraces agents which bind selectively to the Ca_{V}2.2e[37a] molecules (having or encoding SEQ ID NO:45) and agents which bind preferentially to the Ca_{V}2.2e[37a] (having or encoding SEQ ID NO:45) as well as agents which bind to variants and fragments of the polypeptides and nucleic acids as described herein. Selective binding means that the agent binds to the Ca_{V}2.2e[37a] but not to N-type calcium channel non-Ca_{V}2.2e[37a] subunits (i.e., those subunits which do not have or encode SEQ ID NO:45). Preferential binding means that the agent binds more to the Ca_{V}2.2e[37a] than to N-type calcium channel non-Ca_{V}2.2e[37a] subunits, e.g., the agent binds with greater affinity or avidity to the Ca_{V}2.2e[37a] having or encoding SEQ ID NO:45. The agents include polypeptides which bind to Ca_{V}2.2e[37a], antisense nucleic acids, and siRNA molecules. The agents can inhibit or increase Ca_{V}2.2e[37a] activity (antagonists and agonists, respectively).

Some of the agents are inhibitors. A Ca_{V}2.2e[37a] inhibitor is an agent that inhibits Ca_{V}2.2e[37a] mediated voltage gated calcium influx. Ca_{V}2.2e[37a] inhibitors include dominant negative peptides and modified known N-type calcium channel inhibitors including modified ω-conotoxin peptides, such as analogs of ziconotide (SNX-111). Small organic molecule calcium channel inhibitors, such as fluspirilene, NNC 09-0026(−)-trans-1-butyl-4-(4-dimethylaminophenyl)-3-[(4-trifluoromethyl-phenoxy) methyl] piperidinedihydrochloride); SB 201823-A (4-[2-(3,4-dichlorophenoxy)ethyl]-1-pentyl piperidinehydrochloride); NS 649 (2-amino-1-(2,5-dimethoxyphenyl)-5-trifluoromethyl benzimidazole); CNS 1237 (N-acenaphthyl-N′-4-methoxynaphth-1-yl guanidine), riluzole and analogs thereof may also exhibit specificity for the Ca_{V}2.2e[37a].

The invention facilitates the development of analogs of these and other calcium channel inhibitors by providing a specific target molecule that is selectively expressed in nociceptive inhibitors. Useful assays are described below and in the Examples in greater detail. The assays of the invention permit the selection of inhibitors, including those mentioned above, analogs and derivatives of those mentioned above, and other molecules that are selective or preferential antagonists (or agonists) of Ca_{V}2.2e[37a].

Calcium influx assays can be performed to screen and/or determine whether a Ca_{V}2.2e[37a] inhibitor has the ability to inhibit Ca_{V}2.2e[37a] activity, and whether the inhibition is selective or preferential. As used herein, “inhibit” refers to inhibiting by at least 10% voltage gated calcium influx, preferably inhibiting by at least 25% voltage gated calcium influx, and more preferably inhibiting by at least 40% voltage gated calcium influx mediated by Ca_{V}2.2e[37a] as measured by any of the methods well known in the art. An exemplary assay of voltage gated calcium influx is described below in the Examples. By “selective inhibition” is meant that the inhibitor inhibits voltage gated calcium influx mediated by Ca_{V}2.2e[37a] but does not significantly inhibit voltage gated calcium influx mediated by the Ca_{V}2.2e[37b] alternatively spliced subunit. By “preferential inhibition” is meant that the inhibitor inhibits voltage gated calcium influx mediated by Ca_{V}2.2e[37a] by at least about 5% more that voltage gated calcium influx mediated by the Ca_{V}2.2e[37b] alternatively spliced subunit. Preferably, the preferential inhibition is at least about 10% more for Ca_{V}2.2e[37a], more preferably at least about 20% more for Ca_{V}2.2e[37a], still more preferably at least about 30% more for Ca_{V}2.2e[37a], yet more preferably at least about 40% more for Ca_{V}2.2e[37a], and most preferably at least about 50% more for Ca_{V}2.2e[37a]. Greater differences in inhibition of Ca_{V}2.2e[37a] than Ca_{V}2.2e[37b] is contemplated, from 51% all the way up to about 99%, at which point the inhibition may be considered selective.

Inhibitors may selectively or preferentially inhibit Ca_{V}2.2e[37a] based on the state of depolarization of the membrane with which the Ca_{V}2.2e[37a] is associated. It is well known that certain compounds preferentially bind to voltage-gated calcium channels at particular voltages. For example, dihydropyridine compounds preferentially bind to L-type voltage-gated calcium channels when the membrane is depolarized. Bean (Proc. Nat'l. Acad. Sci. 81:6388, 1984) described the binding of nitrendipine to cardiac L-type channels only when the membrane is depolarized. Similar results have been found for nimodipine action in sensory neurons (McCarthy & TanPiengco, J. Neurosci. 12:2225, 1992).

Activators of Ca_{V}2.2e[37a] activity also are enhanced by the invention. Activators may be identified and/or tested using methods described above for inhibitors.

In screening for modulators of Ca_{V}2.2e[37a], including inhibitors and activators (i.e. antagonists and agonists), it is preferred that compounds (e.g., libraries of potential channel inhibitors) are tested for modulation of Ca_{V}2.2e[37a] activity at a variety of voltages which cause partial or complete membrane depolarization, or hyperpolarization. These assays are conducted according to standard procedures of testing calcium channel function (e.g. patch clamping, fluorescent Ca²⁺ influx assays) which require no more than routine experimentation. Using such methods, modulators of Ca_{V}2.2e[37a] activity which are active at particular voltages (e.g., complete membrane depolarization) can be identified. Such compounds are useful for selectively modulating calcium channel activity in conditions which may display voltage dependence. For example, following a stroke membranes are depolarized and such compounds may be active in selectively blocking calcium channel activity for treatment of stroke. Other uses will be apparent to one of ordinary skill in the art.

The invention further provides efficient methods of identifying pharmacological agents or lead compounds for agents useful in the treatment of conditions associated with voltage gated cell calcium influx mediated by calcium channels containing Ca_{V}2.2e[37a] subunits and the compounds and agents so identified. Generally, the screening methods involve assaying for compounds which inhibit or enhance voltage gated calcium influx through human N-type calcium channels. Such methods are adaptable to automated, high throughput screening of compounds. Examples of such methods are described in U.S. Pat. No. 5,429,921.

A variety of assays for pharmacological agents are provided, including, labeled in vitro protein binding assays, Ca²⁺ influx assays, etc. For example, protein binding screens are used to rapidly examine the binding of candidate pharmacological agents to a Ca_{V}2.2e[37a] polypeptide. The candidate pharmacological agents can be derived from, for example, combinatorial peptide libraries, combinatorial chemical compound libraries, and natural products libraries. Convenient reagents for such assays are known in the art. An exemplary cell-based assay of calcium influx involves contacting a neuronal cell that expresses Ca_{V}2.2e[37a] polypeptides in conjunction with other polypeptides that constitute functional N-type calcium channels with a candidate pharmacological agent under conditions whereby the influx of calcium can be stimulated by application of a voltage to the test system, i.e., by membrane depolarization. Specific conditions are well known in the art and are described in Lin et al., Neuron 18:153-166, 1997, and in U.S. Pat. No. 5,429,921. A reduction in the voltage gated calcium influx in the presence of the candidate pharmacological agent indicates that the candidate pharmacological agent reduces the induction of calcium influx in cells expressing the Ca_{V}2.2e[37a] subunit in response to the voltage stimulus. An increase in the voltage gated calcium influx in the presence of the candidate pharmacological agent indicates that the candidate pharmacological agent increases the induction of calcium influx in cells expressing the Ca_{V}2.2e[37a] subunit in response to the voltage stimulus. Methods for determining changes in the intracellular calcium concentration are known in the art and are described elsewhere herein.

Ca_{V}2.2e[37a] molecules used in the methods of the invention can be added to an assay mixture as an isolated polypeptide (where binding of a candidate pharmaceutical agent is to be measured) or as a cell or other membrane-encapsulated space which includes a Ca_{V}2.2e[37a] polypeptide. In the latter assay configuration, the cell or other membrane-encapsulated space can contain the Ca_{V}2.2e[37a] molecule as a preloaded polypeptide or as a nucleic acid (e.g. a cell transfected with an expression vector containing a nucleic acid that encodes a Ca_{V}2.2e[37a] polypeptide). In the assays described herein, the Ca_{V}2.2e[37a] polypeptide can be produced recombinantly, or isolated from biological extracts, but preferably is synthesized in vitro. Ca_{V}2.2e[37a] polypeptides encompass chimeric proteins comprising a fusion of a Ca_{V}2.2e[37a] polypeptide with another polypeptide, e.g., a polypeptide capable of providing or enhancing protein-protein binding, or enhancing stability of the Ca_{V}2.2e[37a] polypeptide under assay conditions. A polypeptide fused to a Ca_{V}2.2e[37a] polypeptide or fragment thereof may also provide means of readily detecting the fusion protein, e.g., by immunological recognition or by fluorescent labeling.

In certain embodiments, the assay includes a control to account for binding and/or modulation of activity of Ca_{V}2.2e[37b] molecules. As above, this can be prepared by expressing a Ca_{V}2.2e[37b] nucleic acid in cells and screening the library of compounds with the cells in parallel with the screening using the Ca_{V}2.2e[37a]-expressing cells. The Ca_{V}2.2e[37b] control cells also can be used to test only positive hit compounds, i.e., those that modulate Ca_{V}2.2e[37a] activity, for their effect on Ca_{V}2.2e[37b] activity. In some instances, the cells can express both Ca_{V}2.2e[37a] and Ca_{V}2.2e[37b] molecules.

For the cell-based assay described herein, preferred cell types are neurons and other cells that permit expression of N-type calcium channels including Ca_{V}2.2e[37a] molecules, including oocytes as shown in the Examples.

The assay mixture also comprises a candidate pharmacological agent. Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection. Candidate agents encompass numerous chemical classes, although typically they are organic compounds. Preferably, the candidate pharmacological agents are small organic compounds, i.e., those having a molecular weight of more than 50 yet less than about 2500. Candidate agents comprise functional chemical groups necessary for structural interactions with polypeptides, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid, the agent typically is a DNA or RNA molecule, although modified nucleic acids having non-natural bonds or subunits are also contemplated. Thus, antisense and siRNA molecules can be tested for inhibition of Ca_{V}2.2e[37a] expression by these assays and other standard assays of nucleic acid expression, such as PCR. Utilizing the cell-based assays described above allows the identification of antisense and siRNA molecules that inhibit function of Ca_{V}2.2e[37a].

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily modified through conventional chemical, physical, and biochemical means. Further, known pharmacological agents may be subjected to directed or random chemical modifications such as acylation, alkylation, esterification, amidification, etc. to produce structural analogs of the agents.

Candidate agents can be selected randomly or can be based on existing compounds which bind to and/or modulate the function of N-type calcium channels. For example, compounds which are known to inhibit N-type calcium channels include fluspirilene, ziconotide (SNX-111), the ω-conotoxin peptides, as well as small organic molecule calcium channel inhibitors, such as fluspirilene, NNC 09-0026(−)-trans-1-butyl-4-(4-dimethylaminophenyl)-3-[(4-trifluoromethyl-phenoxy) methyl] piperidinedihydrochloride); SB 201823-A (4-[2-(3,4-dichlorophenoxy)ethyl]-1-pentyl piperidinehydrochloride); NS 649 (2-amino-1-(2,5-dimethoxyphenyl)-5-trifluoromethyl benzimidazole); CNS 1237 (N-acenaphthyl-N′-4-methoxynaphth-1-yl guanidine) and riluzole. Therefore, a source of candidate agents are libraries of molecules based on the foregoing N-type calcium channel inhibitors, in which the structure of the inhibitor is changed at one or more positions of the molecule to contain more or fewer chemical moieties or different chemical moieties. The structural changes made to the molecules in creating the libraries of analog inhibitors can be directed, random, or a combination of both directed and random substitutions and/or additions. One of ordinary skill in the art in the preparation of combinatorial libraries can readily prepare such libraries based on the existing N-type calcium channel inhibitors.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

The mixture of the foregoing assay materials is incubated under conditions whereby, but for the presence of the candidate pharmacological agent, N-type calcium channels containing the Ca_{V}2.2e[37a] subunit transduces a control amount of voltage gated calcium influx. For determining the binding of a candidate pharmaceutical agent to a Ca_{V}2.2e[37a] molecule, the mixture is incubated under conditions which permit binding. The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 1 minute and 10 hours.

After incubation, the level of voltage gated calcium influx or the level of specific binding between the Ca_{V}2.2e[37a] polypeptide and the candidate pharmaceutical agent is detected by any convenient method available to the user. For cell free binding type assays, a separation step is often used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximize signal to noise ratios, primarily to minimize background binding, as well as for ease of separation and cost.

Separation may be effected for example, by removing a bead or dipstick from a reservoir, emptying or diluting a reservoir such as a microtiter plate well, rinsing a bead, particle, chromatographic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet.

Detection may be effected in any convenient way for cell-based assays such as a calcium influx assay. The calcium influx resulting from voltage stimulus of a N-type calcium channel containing the Ca_{V}2.2e[37a] polypeptide typically alters a directly or indirectly detectable product, e.g., a calcium sensitive molecule such as fura-2-AM. For cell free binding assays, one of the components usually comprises, or is coupled to, a detectable label. A wide variety of labels can be used, such as those that provide direct detection (e.g., radioactivity, luminescence, optical or electron density, etc). or indirect detection (e.g., epitope tag such as the FLAG epitope, enzyme tag such as horseradish peroxidase, etc.). The label may be bound to a Ca_{V}2.2e[37a] polypeptide or the candidate pharmacological agent.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, streptavidin-biotin conjugates, etc. Methods for detecting the labels are well known in the art.

Thus the invention provides numerous assays using Ca_{V}2.2e[37a] molecules to identify modulators, preferably inhibitors, of Ca_{V}2.2e[37a] function. In one embodiment the. Ca_{V}2.2e[37a] inhibitor is an antisense oligonucleotide or siRNA molecule that selectively binds to a Ca_{V}2.2e[37a] nucleic acid molecule, to reduce the expression of Ca_{V}2.2e[37a] in a cell. This is desirable in virtually any medical condition wherein a reduction of Ca_{V}2.2e[37a] activity is desirable, e.g., pain.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

As used herein, a “siRNA molecule” is a double stranded RNA molecule (dsRNA) consisting of a sense and an antisense strand, which are complementary (Tuschl, T. et al., 1999, Genes & Dev., 13:3191-3197; Elbashir, S. M. et al., 2001, EMBO J., 20:6877-6888). In one embodiment the last nucleotide at the 3′ end of the antisense strand may be any nucleotide and is not required to be complementary to the region of the target gene. The siRNA molecule may be 19-23 nucleotides in length in some embodiments. In other embodiments, the siRNA is longer but forms a hairpin structure of 19-23 nucleotides in length. In still other embodiments, the siRNA is formed in the cell by digestion of double stranded RNA molecule that is longer than 19-23 nucleotides. The siRNA molecule preferably includes an overhang on one or both ends, preferably a 3′ overhang, and more preferably a two nucleotide 3′ overhang on the sense strand. In another preferred embodiment, the two nucleotide overhang is thymidine-thymidine (TT). The siRNA molecule corresponds to at least a portion of the Ca_{V}2.2e[37a] gene, but preferably a portion that includes or overlaps at least a portion of exon 37. In a preferred embodiment the first nucleotide of the siRNA molecule is a purine. Many variations of siRNA and other double stranded RNA molecules useful for RNAi inhibition of gene expression will be known to one of ordinary skill in the art.

The siRNA molecules can be plasmid-based. In a preferred method, a polypeptide encoding sequence of Ca_{V}2.2e[37a] is amplified using the well known technique of polymerase chain reaction (PCR). The use of the entire polypeptide encoding sequence is not necessary; as is well known in the art, a portion of the polypeptide encoding sequence is sufficient for RNA interference. For example, the PCR fragment can be inserted into a vector using routine techniques well known to those of skill in the art. The insert can be placed between two promoters oriented in opposite directions, such that two complementary RNA molecules are produced that hybridize to form the siRNA molecule. Alternatively, the siRNA molecule is synthesized as a single RNA molecule that self-hybridizes to form a siRNA duplex, preferably with a non-hybridizing sequence that forms a “loop” between the hybridizing sequences. Preferably the nucleotide encoding sequence is part of the coding sequence of Ca_{V}2.2e[37a], specifically exon 37a to confer specificity. The siRNA can be expressed from a vector introduced into cells.

Vectors comprising any of the nucleotide coding sequences of the invention are provided for production of siRNA, preferably vectors that include promoters active in mammalian cells. Non-limiting examples of vectors are the pSUPER RNAi series of vectors (Brummelkamp, T. R. et al., 2002, Science, 296:550-553; available commercially from OligoEngine, Inc., Seattle, Wash.). In one embodiment a partially self-complementary nucleotide coding sequence can be inserted into the mammalian vector using restriction sites, creating a stem-loop structure. In a preferred embodiment, the mammalian vector comprises the polymerase-III H1-RNA gene promoter. The polymerase-III H1-RNA promoter produces a RNA transcript lacking a polyadenosine tail and has a well-defined start of transcription and a termination signal consisting of five thymidines (T5) in a row. The cleavage of the transcript at the termination site occurs after the second uridine and yields a transcript resembling the ends of synthetic siRNAs containing two 3′ overhanging T or U nucleotides. Other promoters useful in siRNA vectors will be known to one of ordinary skill in the art.

Vector systems for siRNA expression in mammalian cells include pSUPER RNAi system described above. Other examples include but are not limited to pSUPER.neo, pSUPER.neo+gfp and pSUPER.puro (OligoEngine, Inc.); BLOCK-iT T7-TOPO linker, pcDNA1.2/V5-GW/lacZ, pENTRIU6, pLenti6-GW/U6-laminshrna and pLenti6/BLOCK-iT-DEST (Invitrogen). These vectors and others are available from commercial suppliers.

It is preferred that the antisense oligonucleotide or siRNA molecule be constructed and arranged so as to bind selectively with the target under physiological conditions, i.e., to hybridize substantially more to the target sequence than to any other sequence in the target cell under physiological conditions. Based upon sequences encoding Ca_{V}2.2e[37a] polypeptides, or upon allelic or homologous genomic and/or cDNA sequences, one of skill in the art can easily choose and synthesize any of a number of appropriate antisense or siRNA molecules for use in accordance with the present invention. In order to be sufficiently selective and potent for inhibition, such antisense oligonucleotides should comprise at least and, more preferably, at least 15 consecutive bases which are complementary to the target, although in certain cases modified oligonucleotides as short as 7 bases in length have been used successfully as antisense oligonucleotides (Wagner et al., Nature Biotechnol. 14:840-844, 1996). Most preferably, the antisense oligonucleotides comprise a complementary sequence of 20-30 bases. For siRNA molecules, it is preferred that the molecules be 21-23 nucleotides in length, with a 3′ 2 nucleotide overhang, although shorter and longer molecules and molecules without overhangs are also contemplated as usefull in accordance with the inveniton.

In accordance with the identification of Ca_{V}2.2e[37a] as selectively or preferentially expressed in nociceptive neurons, oligonucleotides useful in selectively reducing expression of Ca_{V}2.2e[37a] are chosen to be complementary to the region of the mRNA transcripts that encode exon 37a for the preparation of antisense or siRNA molecules. In addition, the antisense is targeted, preferably, to sites in which mRNA secondary structure is not expected (see, e.g., Sainio et al., Cell Mol. Neurobiol. 14(5):439-457, 1994) and at which polypeptides are not expected to bind. Other methods for selecting preferred siRNA sequences are known to those of skill in the art (e.g., the “siRNA Selection Program” of the Whitehead Institute for Biomedical Research (2003)).

In one set of embodiments, the antisense oligonucleotides or siRNA molecules of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors, including in situ.

In preferred embodiments, however, the antisense oligonucleotides or siRNA molecules of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose. The present invention, thus, contemplates pharmaceutical preparations containing modified antisense molecules that are complementary to and hybridizable with, under physiological conditions, nucleic acids encoding Ca_{V}2.2e[37a] polypeptides, together with pharmaceutically acceptable carriers.

Antisense oligonucleotides or siRNA molecules may be administered as part of a pharmaceutical composition. Such a pharmaceutical composition may include the antisense oligonucleotides or siRNA molecules in combination with any standard pharmaceutically acceptable carriers which are known in the art. The compositions should be sterile and contain a therapeutically effective amount of the antisense oligonucleotides or siRNA molecules in a unit of weight or volume suitable for administration to a patient. The term “pharmaceutically acceptable” means a non-toxic material that does not interfere with the effectiveness of the biological activity of the active ingredients. The characteristics of the carrier will depend on the route of administration. Pharmaceutically acceptable carriers include diluents, fillers, salts, buffers, stabilizers, solubilizers, and other materials which are well known in the art.

Agents which bind Ca_{V}2.2e[37a] also include binding peptides which bind to the Ca_{V}2.2e[37a] and complexes containing the Ca_{V}2.2e[37a]. When the binding polypeptides are inhibitors, the polypeptides bind to and inhibit the activity of Ca_{V}2.2e[37a]. To determine whether a Ca_{V}2.2e[37a] binding peptide binds to Ca_{V}2.2e[37a], any known binding assay may be employed. For example, the peptide may be immobilized on a surface and then contacted with a labeled Ca_{V}2.2e[37a] polypeptide. The amount of Ca_{V}2.2e[37a] which interacts with the Ca_{V}2.2e[37a] binding peptide or the amount which does not bind to the Ca_{V}2.2e[37a] binding peptide may then be quantitated to determine whether the Ca_{V}2.2e[37a] binding peptide binds to Ca_{V}2.2e[37a]. Further, the binding of a Ca_{V}2.2e[37a] polypeptide and a Ca_{V}2.2e[37b] peptide can be compared to determine if the binding peptide binds selectively or preferentially.

The Ca_{V}2.2e[37a] binding peptides include peptides of numerous size and type that bind selectively or preferentially to Ca_{V}2.2e[37a] polypeptides, and complexes of both Ca_{V}2.2e[37a] polypeptides and their binding partners. These peptides may be derived from a variety of sources. For example, Ca_{V}2.2e[37a] binding peptides may include known N-type calcium channel inhibitors such as the ω-conotoxin peptides or modified variants of the foregoing peptides. Binding peptides for Ca_{V}2.2e[37a] polypeptides also can be identified by screening degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to the Ca_{V}2.2e[37a] polypeptide. This process can be repeated through several cycles of reselection of phage that bind to the Ca_{V}2.2e[37a] polypeptide. Repeated rounds lead to enrichment of phage bearing particular sequences. DNA sequence analysis can be conducted to identify the sequences of the expressed polypeptides. The minimal linear portion of the sequence that binds to the Ca_{V}2.2e[37a] polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the Ca_{V}2.2e[37a] polypeptides. Thus, the Ca_{V}2.2e[37a] polypeptides of the invention, or a fragment thereof, can be used to screen peptide libraries, including phage display libraries, to identify and select peptide binding partners of the Ca_{V}2.2e[37a] polypeptides of the invention. Such molecules can be used as described, for screening assays, for purification protocols, for interfering directly with the functioning of Ca_{V}2.2e[37a] and for other purposes that will be apparent to those of ordinary skill in the art.

Peptides may easily be synthesized or produced by recombinant means by those of skill in the art. Using routine procedures known to those of ordinary skill in the art, one can determine whether a peptide which binds to Ca_{V}2.2e[37a] is useful according to the invention by determining whether the peptide is one which inhibits the activity of Ca_{V}2.2e[37a] such as in a voltage gated calcium influx assay, as discussed herein.

The Ca_{V}2.2e[37a] binding peptide agent may also be an antibody or a functionally active antibody fragment. Antibodies are well known to those of ordinary skill in the science of immunology. As used herein, the term “antibody” means not only intact antibody molecules but also fragments of antibody molecules retaining Ca_{V}2.2e[37a] binding ability (antigen binding fragments). Such fragments are also well known in the art and are regularly employed both in vitro and in vivo. In particular, as used herein, the term “antibody” means not only intact immunoglobulin molecules but also well-known active fragments such as F(ab′)₂ and Fab and Fv (including single chain antibodies).

An antibody refers to a glycoprotein comprising at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding fragment” of an antibody as used herein, refers to one or more portions of an antibody that retain the ability to specifically bind to an antigen. It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546) which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference as well as by other techniques known to those with skill in the art. The fragments are screened for utility in the same manner as are intact antibodies.

An “isolated antibody”, as used herein, is intended to refer to an antibody which is substantially free of other antibodies having different antigenic specificities (e.g., a population of isolated antibodies that specifically binds to Ca_{V}2.2e[37a], is substantially free of antibodies that specifically bind antigens other than a Ca_{V}2.2e[37a] molecule). An isolated antibody that specifically binds to an epitope, isoform or variant of Ca_{V}2.2e[37a] may, however, have cross-reactivity to other related antigens, e.g., from other species (e.g., species homologs). Moreover, an isolated antibody may be substantially free of other cellular material and/or chemicals. As used herein, “specific binding” refers to antibody binding to a predetermined antigen. Typically, the antibody binds Ca_{V}2.2e[37a] or a closely-related antigen with an affinity that is at least two-fold greater than its affinity for binding to a non-specific antigen (e.g., BSA, casein) or Ca_{V}2.2e[37b].

The isolated antibodies of the invention encompass various antibody isotypes, such as IgG1, IgG2, IgG3, IgG4, IgM, IgA1, IgA2, IgAsec, IgD, IgE. As used herein, “isotype” refers to the antibody class (e.g. IgM or IgG1) that is encoded by heavy chain constant region genes. The antibodies can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, a F(ab′)₂ fragment, and a Fv fragment.

As used herein, antibodies also include single chain antibodies (e.g., scFvs). In some embodiments, the single chain antibodies are disulfide-free antibodies having mutations e.g., in disulfide bond forming cysteine residues. The antibodies may be prepared by starting with any of a variety of methods, including administering Ca_{V}2.2e[37a]protein, fragments of Ca_{V}2.2e[37a] protein that include SEQ ID NO:45, cells expressing the protein or fragments thereof and the like to an animal to induce polyclonal antibodies. Such antibodies or antigen-binding fragments thereof may be used in the preparation of scFvs and disulfide-free variants thereof. The antibodies or antigen-binding fragments thereof may be used for example to modulate the activity of a target protein (e.g. Ca_{V}2.2e[37a]).

Various forms of the antibody polypeptide or encoding nucleic acid can be administered and delivered to a mammalian cell (e.g., by virus or liposomes, or by any other suitable methods known in the art). The method of delivery can be modified to target certain cells, and in particular, cell surface receptor molecules or antigens present on specific cell types. Methods of targeting cells to deliver nucleic acid constructs, for intracellular expression of the antibodies, are known in the art. The antibody polypeptide sequence can also be delivered into cells by expressing a recombinant protein fused with peptide carrier molecules. These carrier molecules, which are also referred to herein as protein transduction domains (PTDs), and methods for their use, are known in the art. Examples of PTDs, though not intended to be limiting, are tat, antennapedia, and synthetic poly-arginine. These delivery methods are known to those of skill in the art and are described in U.S. Pat. No. 6,080,724, and U.S. Pat. No. 5,783,662, the entire contents of which are hereby incorporated by reference.

The antibodies of the present invention can be polyclonal, monoclonal, or a mixture of polyclonal and monoclonal antibodies. The antibodies can be produced by a variety of techniques well known in the art. Procedures for raising polyclonal antibodies are well known and are disclosed for example in E. Harlow, et. al., editors, Antibodies: A Laboratory Manual (1988), which is hereby incorporated by reference.

Monoclonal antibody production may be effected by techniques which are also well known in the art. The term “monoclonal antibody,” as used herein, refers to a preparation of antibody molecules of single molecular composition. A monoclonal antibody displays a single binding specificity and affinity for a particular epitope. The process of monoclonal antibody production involves obtaining immune somatic cells with the potential for producing antibody, in particular B lymphocytes, which have been previously immunized with the antigen of interest either in vivo or in vitro and that are suitable for fusion with a B-cell myeloma line.

Mammalian lymphocytes typically are immunized by in vivo immunization of the animal (e.g., a mouse) with the desired protein or polypeptide, e.g., with a Ca_{V}2.2e[37a] polypeptide. Such immunizations are repeated as necessary at intervals of up to several weeks to obtain a sufficient titer of antibodies. Once immunized, animals can be used as a source of antibody-producing lymphocytes. Following the last antigen boost, the animals are sacrificed and spleen cells removed. See; Goding (in Monoclonal Antibodies: Principles and Practice, 2d ed., pp. 60-61, Orlando, Fla., Academic Press, 1986).

The antibody-secreting lymphocytes are then fused with (mouse) B cell myeloma cells or transformed cells, which are capable of replicating indefinitely in cell culture, thereby producing an immortal, immunoglobulin-secreting cell line. The resulting fused cells, or hybridomas, are cultured, and the resulting colonies screened for the production of the desired monoclonal antibodies. Colonies producing such antibodies are cloned, and grown either in vivo or in vitro to produce large quantities of antibody. A description of the theoretical basis and practical methodology of fusing such cells is set forth in Kohler and Milstein, Nature 256:495 (1975), which is hereby incorporated by reference.

Myeloma cell lines suited for use in hybridoma-producing fusion procedures preferably are non-antibody-producing, have high fusion efficiency, and enzyme deficiencies that render them incapable of growing in certain selective media which support the growth of the desired hybridomas. Examples of such myeloma cell lines that may be used for the production of fused cell lines include P3-X63/Ag8, X63-Ag8.653, NS1/1.Ag 4.1, Sp2/0-Ag14, FO, NSO/U, MPC-11, MPC11-X45-GTG 1.7, S194/5XX0 Bul, all derived from mice; R210.RCY3, Y3-Ag 1.2.3, IR983F and 4B210 derived from rats and U-266, GM1500-GRG2, LICR-LON-HMy2, UC729-6, all derived from humans (Goding, in Monoclonal Antibodies: Principles and Practice, 2d ed., pp. 65-66, Orlando, Fla., Academic Press, 1986; Campbell, in Monoclonal Antibody Technology, Laboratory Techniques in Biochemistry and Molecular Biology Vol. 13, Burden and Von Knippenberg, eds. pp. 75-83, Amsterdam, Elseview, 1984).

Fusion with mammalian myeloma cells or other fusion partners capable of replicating indefinitely in cell culture is effected by standard and well-known techniques, for example, by using polyethylene glycol (“PEG”) or other fusing agents (See Milstein and Kohler, Eur. J. Immunol. 6:511 (1976), which is hereby incorporated by reference).

In other embodiments, the antibodies can be recombinant antibodies. The term “recombinant antibody”, as used herein, is intended to include antibodies that are prepared, expressed, created or isolated by recombinant means, such as antibodies isolated from an animal (e.g., a mouse) that is transgenic for another species' immunoglobulin genes, antibodies expressed using a recombinant expression vector transfected into a host cell, antibodies isolated from a recombinant, combinatorial antibody library, or antibodies prepared, expressed, created or isolated by any other means that involves splicing of immunoglobulin gene sequences to other DNA sequences.

In yet other embodiments, the antibodies can be chimeric or humanized antibodies. As used herein, the term “chimeric antibody” refers to an antibody, that combines the murine variable or hypervariable regions with the human constant region or constant and variable framework regions. As used herein, the term “humanized antibody” refers to an antibody that retains only the antigen-binding CDRs from the parent antibody in association with human framework regions (see, Waldmann, 1991, Science 252:1657). Such chimeric or humanized antibodies retaining binding specificity of the murine antibody are expected to have reduced immunogenicity when administered in vivo for diagnostic, prophylactic or therapeutic applications according to the invention.

In certain embodiments, the antibodies are human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse have been grafted onto human framework sequences (referred to herein as “humanized antibodies”). Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals results in the production of fully human antibodies to the antigen of interest. Following immunization of these mice (e.g., XenoMouse (Abgenix), HuMAb mice (Medarex/GenPharm)), monoclonal antibodies are prepared according to standard hybridoma technology. These monoclonal antibodies have human immunoglobulin amino acid sequences and therefore will not provoke human anti-mouse antibody (HAMA) responses when administered to humans. In particular, mouse strains that have human immunoglobulin genes inserted in the genome (and which cannot produce mouse immunoglobulins) are preferred. Such mice produce fully human immunoglobulin molecules in response to immunization.

According to the invention Ca_{V}2.2e[37a] inhibitors also include “dominant negative” polypeptides. A dominant negative polypeptide is an inactive variant of a polypeptide, which, by interacting with the cellular machinery, displaces an active polypeptide from its interaction with the cellular machinery or competes with the active polypeptide, thereby reducing the effect of the active polypeptide. For example, a dominant negative Ca_{V}2.2e[37a] subunit of an active complex (e.g. N-type calcium channel) can interact with the complex but prevent the activity of the complex (e.g., voltage gated calcium influx).

Alternatively, non-human transgenic animal can be prepared using the Ca_{V}2.2e[37a] nucleic acid described herein. A “transgenic animal” is an animal having cells that contain DNA which has been artificially inserted into a cell, which DNA becomes part of the genome of the animal which develops from that cell, or which contain nucleic acid molecules that are expressed in a cell transiently. Preferred transgenic animals are primates, mice, rats, cows, pigs, horses, goats, sheep, dogs and cats. A variety of methods for the production of transgenic animals are well known in the art. Animals suitable for transgenic experiments can be obtained from standard commercial sources such as Charles River (Wilmington, Mass.), Taconic (Germantown, N.Y.), Harlan Sprague Dawley (Indianapolis, Ind.), etc. Transgenic animals having a particular property associated with a particular disease can be used to study the affects of a variety of drugs and treatment methods on the disease, and thus serve as genetic models for the study of a number of human diseases. The invention, therefore, contemplates the use of Ca_{V}2.2e[37a] transgenic animals as models for the study of disorders involving voltage gated calcium influx in nociceptive neurons.

Inactivation or replacement of exon 37a of the endogenous Ca_{V}2.2 subunit gene can be achieved, for example, by a homologous recombination system using embryonic stem cells. The resultant transgenic non-human mammals (preferably primates) having a knockout Ca_{V}2.2e[37a] characteristic may be used as a model for the effects of compounds that selectively or preferentially inhibit Ca_{V}2.2e[37a] activity. A similar effect can be achieved by testing the antisense or siRNA molecules described herein in order to prepare “knockdown” animals in which expression of Ca_{V}2.2e[37a] is reduced or eliminated. Such animals also can be used to assess the effects of antisense or siRNA therapies.

The compositions of the invention are also useful for therapeutic purposes. Accordingly the invention encompasses a method for modulating (reducing or increasing) Ca_{V}2.2e[37a] activity in a mammalian cell. In preferred embodiments, the method involves contacting the mammalian cell with an amount of a Ca_{V}2.2e[37a] inhibitor effective to inhibit voltage gated calcium influx in the mammalian cell. Such methods are useful in vitro for altering voltage gated calcium influx for the purpose of, for example, elucidating the mechanisms involved in pain, e.g., neuropathic pain, and for restoring normal voltage gated calcium influx in a cell having a defective (excessively active) Ca_{V}2.2e[37a]. In vivo, such methods are useful, for example, for reducing N-type voltage gated calcium influx, e.g., to treat pain, e.g., neuropathic pain, or any condition in which Ca_{V}2.2e[37a] activity is elevated.

An amount of a Ca_{V}2.2e[37a] inhibitor which is effective to inhibit voltage gated calcium influx in the mammalian cell is an amount which is sufficient to reduce voltage gated calcium influx by at least 10%, preferably at least 20%, more preferably 30% and still more preferably 40%. An amount of a Ca_{V}2.2e[37a] which is effective to increase voltage gated calcium influx in the mammalian cell is an amount which is sufficient to increase voltage gated calcium influx by at least 10%, preferably at least 20%, more preferably 30% and still more preferably 40%. Such alterations in voltage gated calcium influx can be measured by the assays described herein.

As described above with respect to inhibitors, modulators of Ca_{V}2.2e[37a] may selectively or preferentially inhibit or increase Ca_{V}2.2e[37a] function based on the state of depolarization of the membrane with which the Ca_{V}2.2e[37a] polypeptide is associated. Therefore, in screening for modulators of Ca_{V}2.2e[37a], it is preferred that compounds (e.g. synthetic combinatorial libraries, natural products, peptide libraries, etc.) are tested for modulation of Ca_{V}2.2e[37a] activity at a variety of voltages which cause partial or complete membrane depolarization, or hyperpolarization. These assays are conducted according to standard procedures of testing calcium channel function (e.g. patch clamping, fluorescent Ca²⁺ influx assays) which require no more than routine experimentation. Using such methods, modulators of Ca_{V}2.2e[37a] activity which are active at particular voltages (e.g. complete membrane depolarization) can be identified. Such compounds are useful for selectively modulating calcium channel activity in conditions which may display voltage dependence.

The invention also encompasses a method for increasing Ca_{V}2.2e[37a] expression in a cell. It is desirable to increase Ca_{V}2.2e[37a] in a cell, for example, to prepare cells useful in the assays described herein. The amount of Ca_{V}2.2e[37a] can be increased in such cell by contacting the cell with a Ca_{V}2.2e[37a] nucleic acid or a Ca_{V}2.2e[37a] polypeptide of the invention in an amount effective to increase voltage gated calcium influx in the cell. An increase in Ca_{V}2.2e[37a] activity can be measured by the assays described herein, e.g., assays of calcium influx.

The preparations of the invention are administered in effective amounts. An effective amount is that amount of a pharmaceutical preparation that alone, or together with further doses, produces the desired response. In the case of treating a condition characterized by aberrant voltage gated calcium influx, the desired response is reducing or increasing calcium influx to a level which is within a normal range. Preferably, the change in calcium influx produces a detectable reduction in a physiological function related to the condition, e.g., a reduction in pain. The responses can be monitored by routine methods. In the case of a condition where an increase in voltage gated calcium influx is desired, an effective amount is that amount necessary to increase said influx in the target tissue. The converse is the case when a reduction in influx is desired. When administered for treatment of pain conditions, the effectiveness of inhibitors of Ca_{V}2.2e[37a] can be assessed directly by querying the patient regarding, e.g., the extent and duration of pain relief.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. It is preferred generally that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Generally, doses of active compounds would be from about 0.01 ng/kg per day to 1000 mg/kg per day. It is expected that doses ranging from 50 g-500 mg/kg will be suitable and in one or several administrations per day. Lower doses can result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compound, although fewer doses typically will be given when compounds are prepared as slow release or sustained release medications.

When administered, the pharmaceutical preparations of the invention are applied in pharmaceutically-acceptable amounts and in pharmaceutically-acceptably compositions. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The Ca_{V}2.2e[37a] inhibitors or Ca_{V}2.2e[37a] nucleic acids and polypeptides useful according to the invention may be combined, optionally, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; and phosphoric acid in a salt.

The pharmaceutical compositions also may contain, optionally, suitable preservatives, such as: benzalkonium chloride; chlorobutanol; parabens and thimerosal.

A variety of administration routes are available. The particular mode selected will depend, of course, upon the particular compound selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intrathecal, intramuscular, or infusion.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous preparation of the Ca_{V}2.2e[37a] inhibitor or Ca_{V}2.2e[37a] nucleic acids and polypeptides, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intrathecal, intramuscular, etc. administrations can be found in Remington's Pharmaceutical Sciences, Mack Publishing Co., Easton, Pa.

Other delivery systems can include time-release, delayed release or sustained release delivery systems. Such systems can avoid repeated administrations of the active compound, increasing convenience to the subject and the physician. Many types of release delivery systems are available and known to those of ordinary skill in the art. Use of a long-term sustained release implant may be desirable. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

The invention will be more fully understood by reference to the following examples. These examples, however, are merely intended to illustrate the embodiments of the invention and are not to be construed to limit the scope of the invention.

### EXAMPLES

### Example 1

### Analysis of N-type Calcium Current and Ca_{V}2.2a1 Splice Variants in Nociceptive Neurons

N-type calcium channels are important for pain transduction. The N-type Ca_{V}2.2 α1 gene contains a number of exons that are subject to tissue-specific alternative splicing (Lipscombe et al., 2002). Dorsal root ganglia (DRG) neurons express multiple Ca_{V}2.2 α1 splice isoforms (Bell et al., 2001). Here we report that exon 37a (e37a), located in the C-terminus of Ca_{V}2.2 α1, is expressed in a subset of nociceptive neurons. E37a encodes 32 amino acids in the C-terminus, is expressed in a mutually exclusive manner with e37b, and disrupts a putative EF-hand motif. The cell-specific expression of e37a prompted us to compare the functional properties of N-type calcium channel currents in dorsal root ganglia. Nociceptive neurons express signature markers such as the non-specific cation channel VR-1 (Caterina et al., 1997). DRG neurons were therefore operationally defined as nociceptive if they responded to the VR-1 receptor ligand capsaicin. We found that capsaicin-responsive neurons containing e37a have a statistically greater peak N-type current density compared to capsaicin-responsive neurons that lack e37a.

Materials and Methods

Calcium Current Recordings

Acutely dissociated DRG neurons were placed into the recording chamber containing Tyrodes solution (140 mM NaCl, 5 mM KCl, 2 mM CaCl₂, 0.5 mM MgCl₂, 10 mM HEPES, and 15 mM Dextrose; the pH was adjusted to 7.2 with NaOH). Patch electrodes were fabricated from borosilicate glass and polished to a resistance of 1-2 MΩ. The internal solution for the perforated patch recordings consisted of 135 mM CsCl, 10 mM HEPES, 1 mM EGTA, 1 mM EDTA, 4 mM MgCl₂ 1.2 pg/ml amphotericin and adjusted to a pH of 7.2 with TEA-OH. Seals were formed in Tyrodes solution. The series resistance decreased over a 20 min period and deemed acceptable when it fell below 12 MΩ. The bath solution was then replaced with 135 mM TEA-Cl, 2 mM CaCl₂, 10 mM HEPES, 100 nM TTX and adjusted to a pH of 7.2 with TEA-OH. Whole cell calcium currents were recorded under constant bath perfusion. A current voltage protocol from a holding potential of −80 mV with 50 ms test pulses was then delivered. ω-Ctx GVIA (3 μM) was then applied via a second pipette using positive pressure. Test pulses at 20 second intervals were applied during toxin application. When toxin block was maximal (three test pulses generating equal sized currents) a second current voltage protocol was delivered. Finally the bath solution was exchanged with Tyrodes and the cell was held at −60 mV. 2 μM capsaicin in Tyrodes was then exposed to the cell. The entire cell was then harvested and stored in first-strand buffer at −80 until needed. After each application of ω-Ctx GVIA, the recording chamber was soaked in 2% SDS and 10 mM DTT for 15 min to remove any residual ω-Ctx GVIA. Data acquisition was performed using pClamp V8.1 software and the Axopatch 200A (Axon Instruments). For recordings of calcium current data was filtered at 2 kHz and sampled at 20 kHz. The series resistance was compensated by 70-90% with a 10 ms lag. Leak correction was performed with a p/−4 protocol. For recordings of VR-1 current data was filtered at 2 kHz and sampled at 2 kHz. No leak subtraction or series resistance compensation was performed. All recordings were obtained at room temperature.

Capsaicin Screening

Viable neurons were identified using whole cell voltage clamp recordings based on the presence of robust voltage-gated sodium and potassium currents. Neurons were then exposed to 2 μM capsaicin (Sigma). After exposure, the bath was washed and the cell allowed to recover. Capsaicin-responsive cells had to recover after wash to be included. Capsaicin-non-responsive neurons were expose at least twice to capsaicin before the cell was collected and counted as capsaicin-non-responsive. Coverslips in the study were limited to two exposures of capsaicin to avoid desensitization. 1-2 MΩ microelectrodes were filled with an internal solution consisting of 90 mM KCl, 10 mM NaCl, 5 mM MgCl₂, 40 mM HEPES, 5 mM EGTA and the pH was adjusted to 7.4 with KOH. The bath solution contained 140 mM NaCl, 5 mM KCl, 2 mM CaCl₂, 0.5 mM MgCl₂, 10 mM HEPES, and 15 mM Dextrose. The pH was adjusted to 7.2 with NaOH. At the conclusion of the recording the entire cell was harvested via the recording pipette by negative pressure and stored in first-strand buffer at −80° C. until needed. Data acquisition was performed using PClamp V8.1 software and Axopatch 200A (Axon Instruments). Data was filtered at 2 kHz and sampled at 2 kHz. No leak correction or series resistance compensation was performed. The recordings were obtained at room temperature.

Single Cell RT-PCR Protocol

The electrode tip and its contents (˜2 μl) were broken into a thin-walled PCR tube containing water and First-strand buffer cDNA was synthesized from single cells according the Superscript II protocol with minor modifications. cDNA from single cells was then used as a template for subsequent PCR reactions with primers for GAPDH (0.1 of cDNA) and Ca_{V}2.2 α1 e37a or e37b (˜0.5 of cDNA). Products were then run out on 2% standard agarose and stained with EthBr. Competitive PCR templates were spiked into PCR reactions for either e37a and e37b. For 1 ng DRG tissue (˜10 cells) competitor dilutions of 10⁻¹⁸-10⁻²² and for single cell cDNA 10-20 were used. Products were then run out on 4% Metaphor agarose gels and stained with EthBr. Band intensities were directly quantified from gels using a digital analysis system (Alpha Innotech).

Results

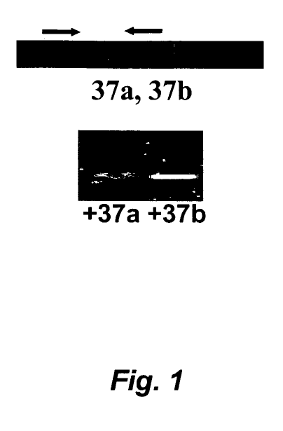

FIG. 1 shows RT-PCR analysis of e37a and e37b splice variants. RT-PCR analysis of e37a and e37b splice variants of Ca_{V}2.2 from rat dorsal root ganglia showed that Ca_{V}2.2 containing exons 37a or e37b were expressed in rat DRG tissue (P5).

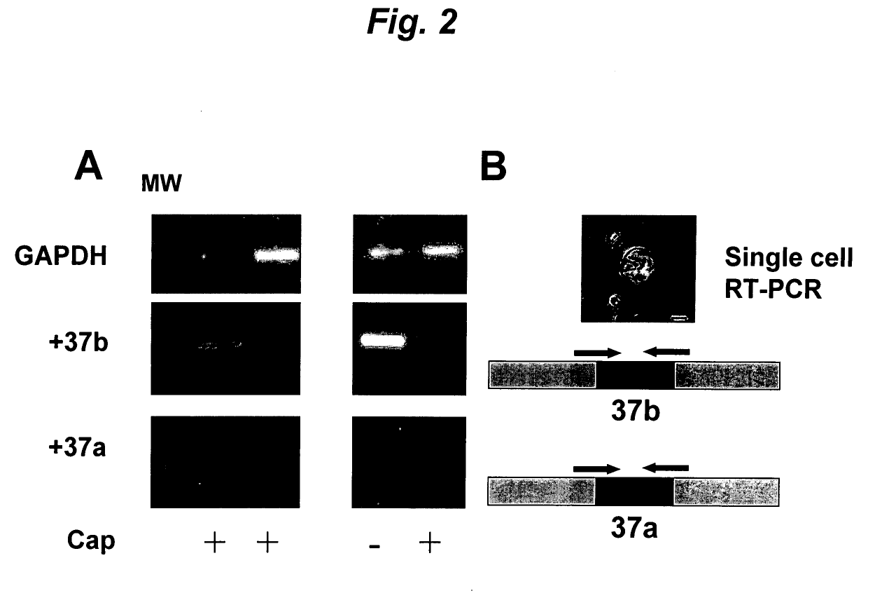

FIG. 2 shows RT-PCR analysis of e37a and e37b in single DRG neurons. The PCR primer pairs were exon-specific for e37a and e37b. The predicted PCR product was 108 bp for both e37a and e37b. FIG. 2A, Representative gel for scRT-PCR for e37a, e37b and GAPDH in the same neuron. FIG. 2B, Diagrams of the PCR primer locations for each site.

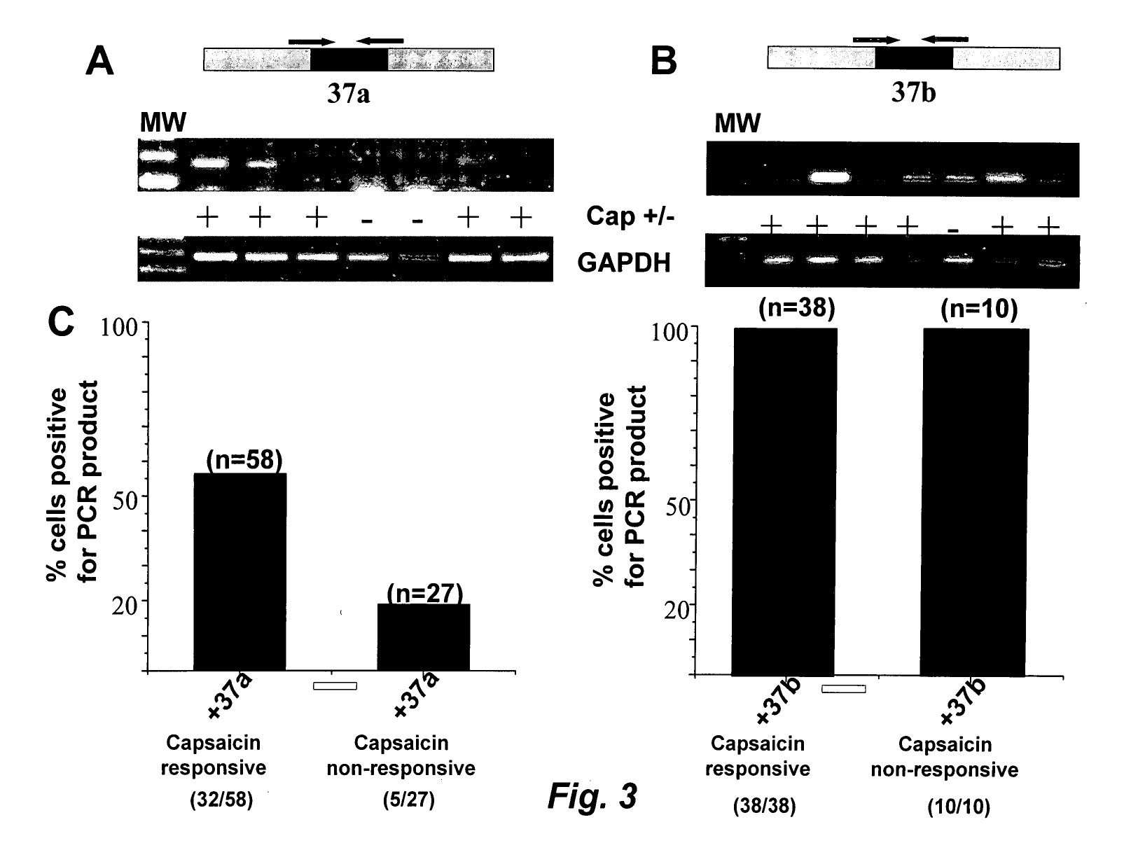

FIG. 3 shows single cell RT-PCR (scRT-PCR) analysis of Ca_{V}2.2 e37a and e37b in capsaicin-responsive and capsaicin-non-responsive neurons. FIG. 3A, Representative gel for scRT-PCR for e37a and GAPDH. FIG. 3B, Representative gel for scRT-PCR for e37b and GAPDH. FIG. 3C, Bar graph summary of e37a and e37b results.

Exon 37a was expressed only in a subset of neurons. e37a PCR products were detected in 55% of capsaicin-responsive neurons (32 of 58) but in only 19% (5 of 27) of non-responsive neurons. This result suggests that exon 37a is expressed in a subset of nociceptive neurons in the DRG.

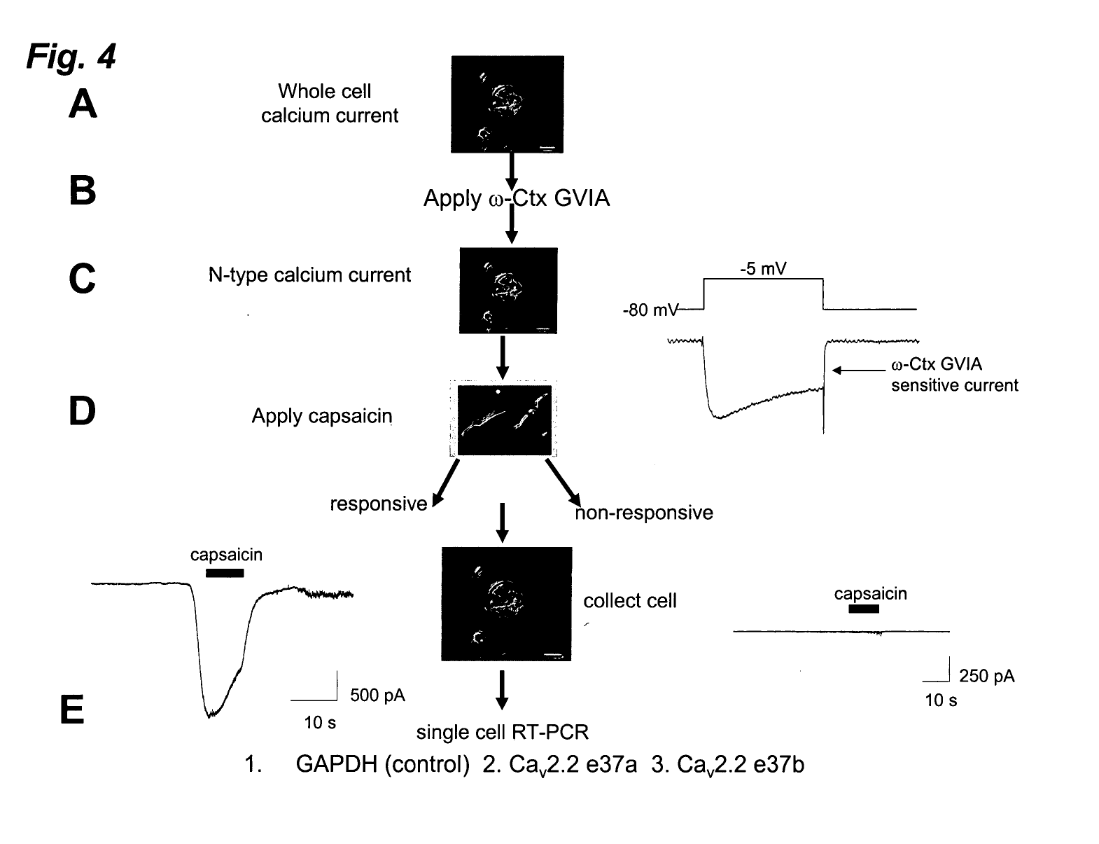

FIG. 4 shows the experimental protocol for certain experiments. DRG neurons from P5-8 rats were dissociated and immediately plated on glass coverslips. FIG. 4A, Whole cell voltage-clamp recordings of calcium currents were performed using the perforated patch technique. Current-voltage curves were obtained from each cell (holding potential=−80 mV). FIG. 4B, 3 mM ω-Ctx GVIA was then applied via a micropipette and the onset of block assessed by test pulses to peak current every 20 seconds. At maximum block a second I-V curve was obtained. FIG. 4C, Post-toxin currents were digitally subtracted from pre-toxin currents to reveal the toxin-sensitive component of the whole cell calcium current. FIG. 4D, Finally, 2 mM capsaicin was applied and the presence of an inward current of at least 25 pA at a holding potential of −60 mV, defined a capsaicin-responsive neuron. FIG. 4E, Neurons were collected for single cell RT-PCR analysis of e37a and 37b of Ca_{V}2.2, and GAPDH.

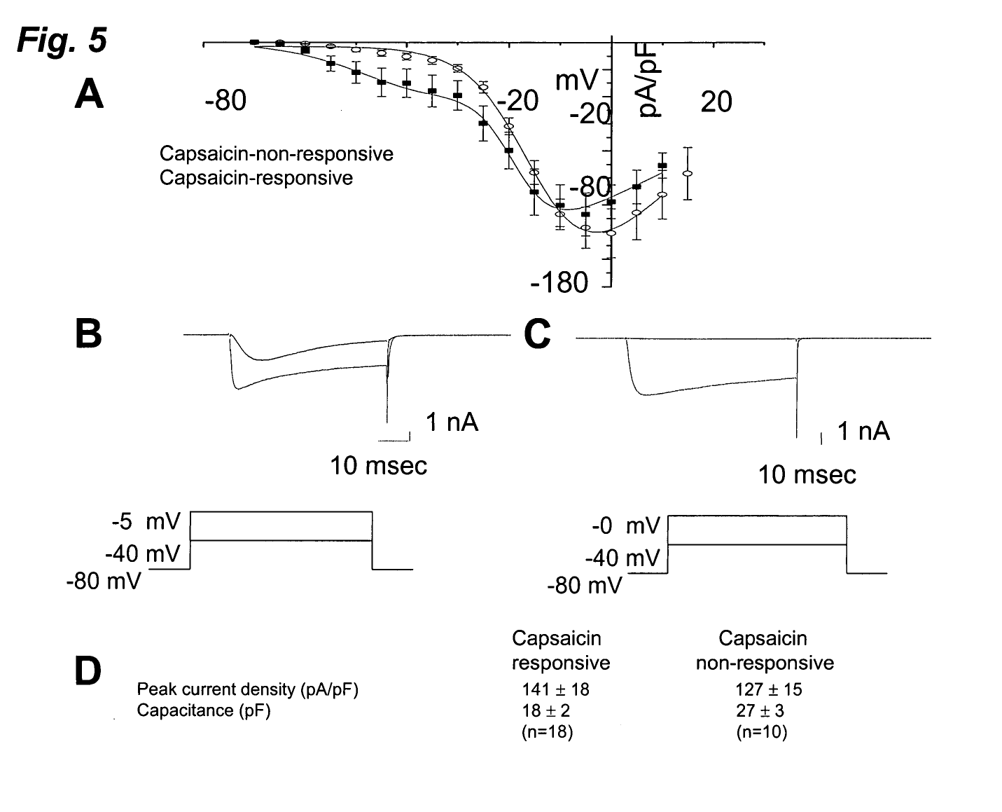

FIG. 5 shows whole cell calcium currents in capsaicin-responsive and capsaicin-non-responsive neurons. FIG. 5A, I-V plots of the whole cell calcium current in capsaicin-responsive (∘) and capsaicin-non-responsive (▪) DRG neurons. FIG. 5B, Representative low voltage-activated (LVA) and high voltage-activated (HVA) whole cell calcium currents from a capsaicin-non-responsive neuron. FIG. 5C, Same as FIG. 5B from a capsaicin-responsive neuron. FIG. 5D, Summary of peak calcium current density and capacitance of capsaicin-responsive (V_{1/2}˜−˜15 mV) and non-responsive neurons (V_{1/2}˜−45 mV and −15 mV for LVA and HVA, respectively. The whole cell capacitance of capsaicin-non-responsive neurons is significantly greater compared to capsaicin-responsive neurons (Students t test; p<0.001).

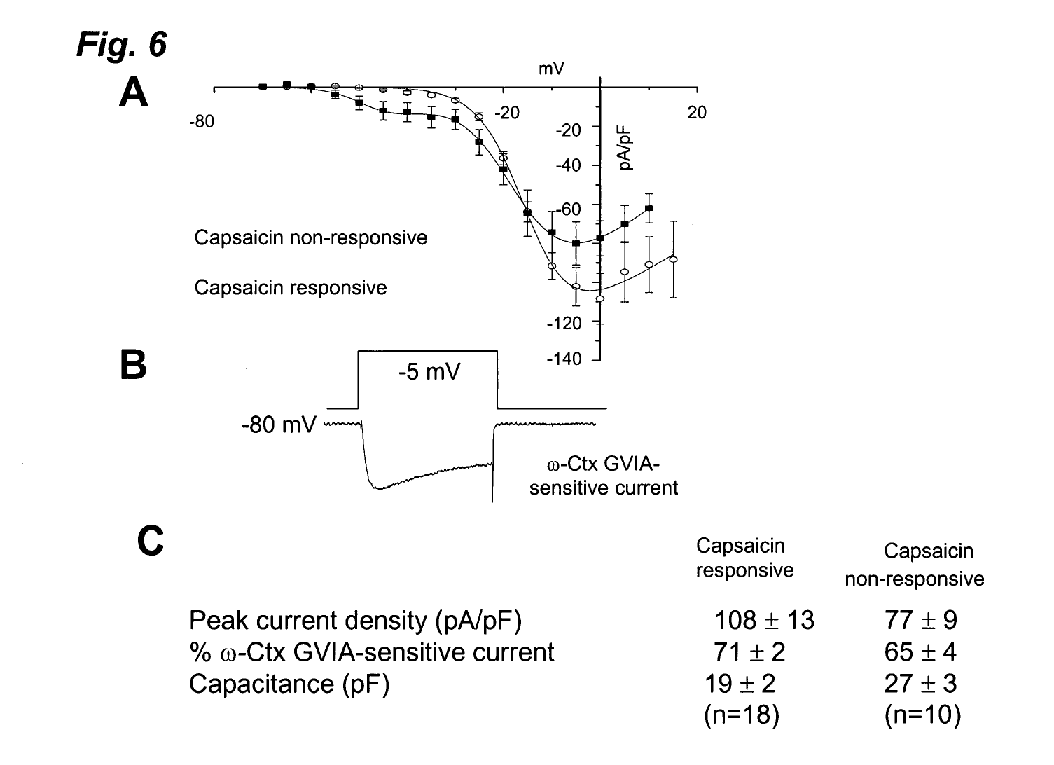

FIG. 6 shows that ω-Ctx GVIA-sensitive calcium currents in capsaicin-responsive and capsaicin-non-responsive neurons. FIG. 6A, I-V plots of ω-Ctx GVIA-sensitive calcium current in capsaicin-responsive (∘) and capsaicin-non-responsive (▪) DRG neurons. V_{1/2}=−15 mV for capsaicin-responsive and V_{1/2}=˜−45 mV and −15 mV for LVA and HVA currents in capsaicin-non-responsive neurons. FIG. 6B, Representative ω-Ctx GVIA-sensitive calcium currents. FIG. 6C, Summary of peak calcium channel current density, % ω-Ctx GVIA-sensitive calcium current, and capacitance in capsaicin-responsive and non-responsive neurons.

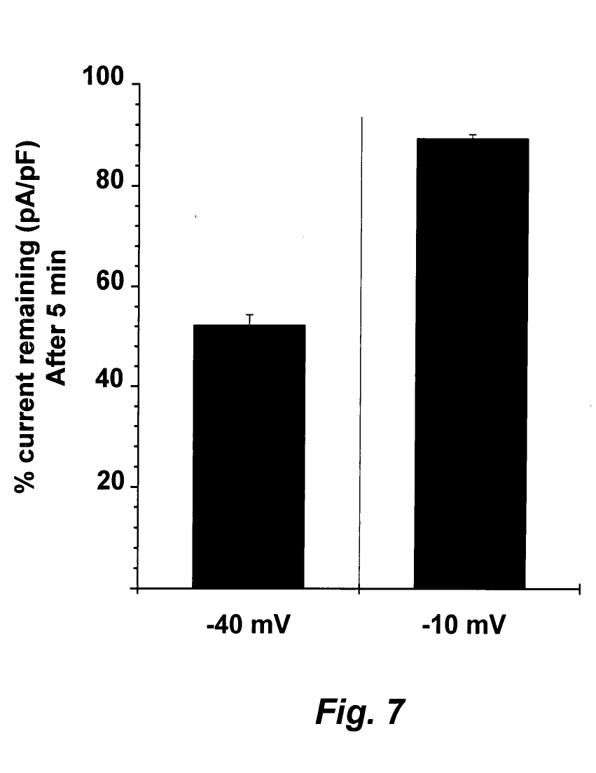

FIG. 7 shows that LVA currents rundown significantly over a 5 minute time period in capsaicin-non-responsive neurons. The presence of a low voltage-activated GVIA-sensitive current in capsaicin-non-responsive cells is surprising (FIG. 6A). GVIA is well known for is selective effects on N-type HVA currents. We therefore tested the hypothesis that the LVA current ran down over the time period of the experiment even in the absence of GVIA. The same voltage protocol used during the toxin application experiments was employed. Only 52±2% of the LVA current (−40 mV) remained after 5 minutes compared to 89±1% of HVA current (−10 mV). The apparent presence of GVIA-sensitive LVA currents in capsaicin-non-responsive neurons in FIG. 6 is simply a function of preferential rundown of LVA currents.

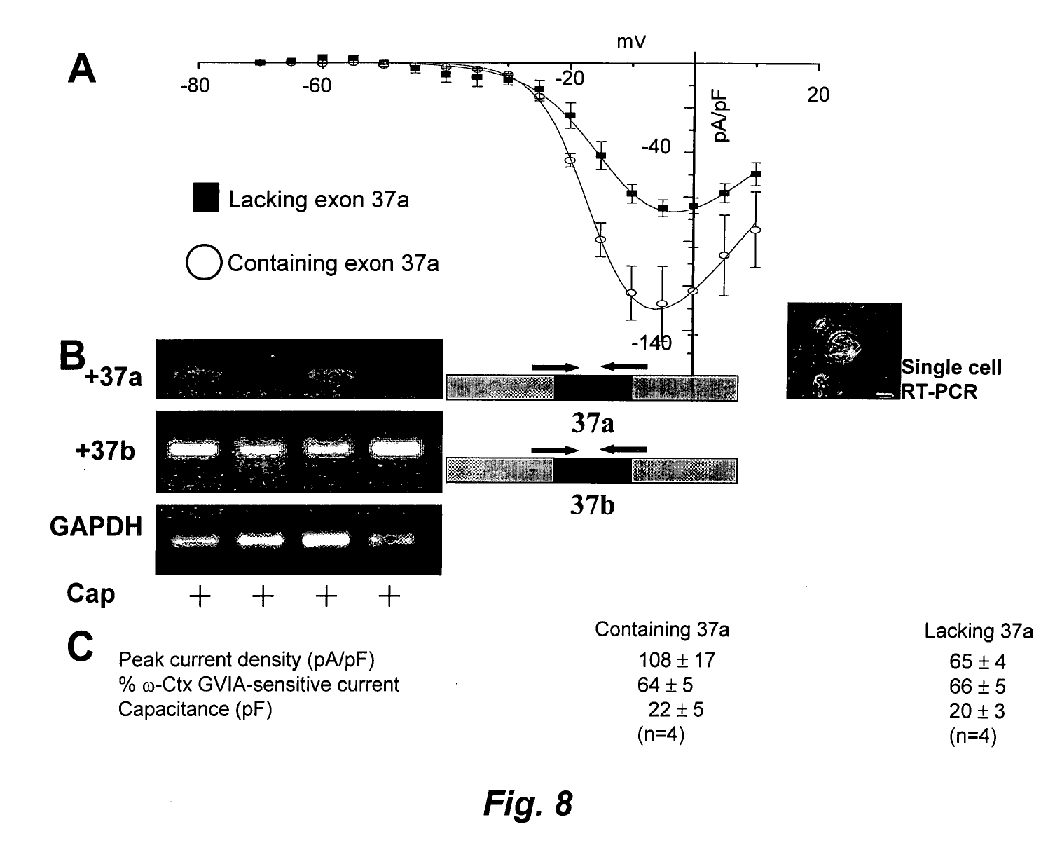

FIG. 8 shows ω-Ctx GVIA-sensitive calcium currents in capsaicin-responsive neurons that contain and lack e37a. FIG. 8A, I-V plots of ω-Ctx GVIA-sensitive calcium currents in capsaicin-responsive neurons that contain (∘) and lack (▪) e37a. FIG. 8B, Representative gel from scRT-PCR for e37a, e37b, and GAPDH in the same neuron. PCR primer locations are shown. FIG. 8C, Summary of GVIA-sensitive peak calcium current density, % ω-Ctx GVIA-sensitive calcium current, and capacitance in capsaicin-responsive and non-responsive neurons. The peak N-type current density was significantly greater in capsaicin-responsive neurons containing e37a compared to exon 37a-lacking (Students t test; p<0.05). V_{1/2}˜−15 mV for both groups.

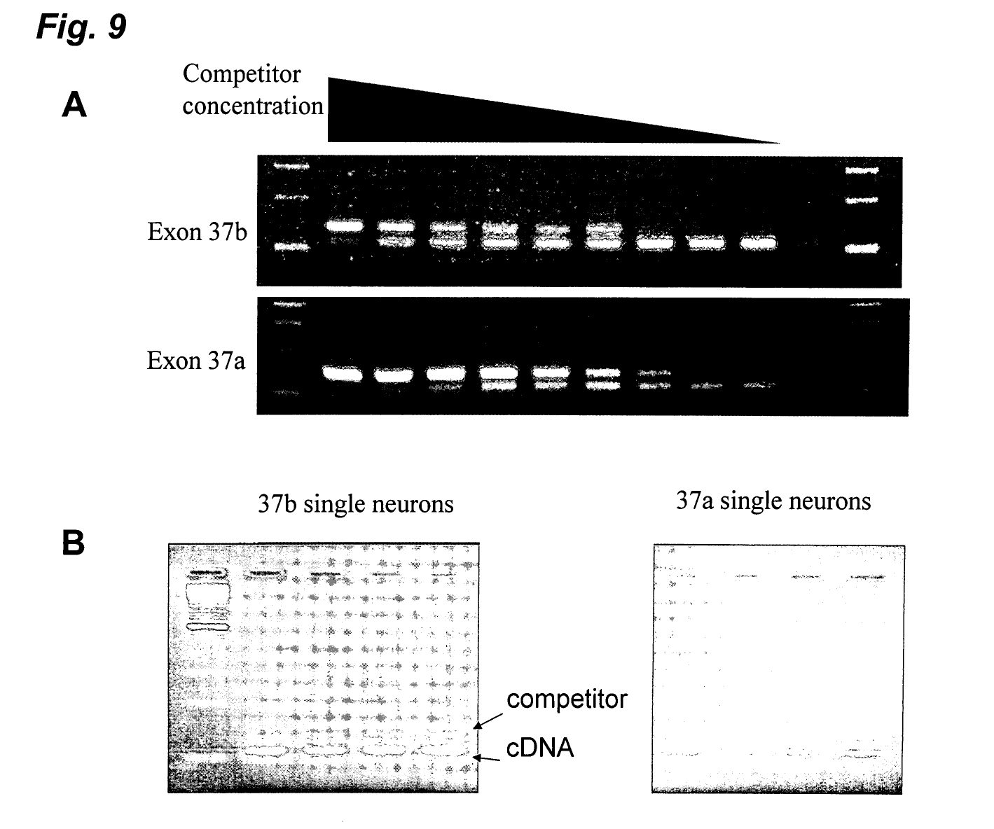

FIG. 9 shows competitive RT-PCR analysis of e37a and e37b in whole tissue and single neurons. Using different exon-specific PCR primers for e37a and e37b, we were not able to determine relative levels of each exon. To estimate approximate levels of e37a and e37b we used a competitive single cell RT-PCR protocol. Each primers pair generated two PCR products; a 108 bp product from endogenous Ca_{V}2.2 cDNA and a 135 bp product from the competitive template added in known amounts to PCR reactions. FIG. 9A, Representative gel for RT-PCR of whole DRG tissue for e37a and e37b in the presence of serial dilutions of competitive template (10⁻¹⁸-10⁻²² M). FIG. 9B, Representative gel for scRT-PCR for e37a and e37b with the same competitive template dilution (10⁻²⁰ M) from the same neuron.

### SUMMARY

Exon 37a of Ca_{V}2.2α1 is restricted to a subset of DRG neurons. 55% of capsaicin-responsive neurons expressed e37a while only 19% of capsaicin-non-responsive neurons e37a.

Analysis of whole cell calcium current revealed that capsaicin-non-responsive neurons express a low voltage-activating current that is less prominent in capsaicin-responsive neurons. The N-type current density was slightly, but not significantly greater in capsaicin-responsive (108±13 pA/pF) compared to non-responsive neurons (77±9 pA/pF)

Capsaicin-responsive neurons that contain e37a have a significantly larger N-type current (108±16 pA/pF) compared to capsaicin-responsive neurons that lack e37a (65±4 pA/pF). Students t test; p<0.05).

Conclusions

We have demonstrated that Ca_{V}2.2α1 splice variants containing e37a are preferentially expressed in a subset of nociceptive neurons of the dorsal root ganglia.

The functional significance of exon 37a is not yet known, however, capsaicin-responsive neurons that contain e37a have a larger N-type current compared to those lacking e37a.

It is possible that e37a promotes surface expression of Ca_{V}2.2. The greater N current density in nociceptive neurons expressing e37a may have significance specific to the physiology of pain.

### Example 2

### Cell-Specific Alternative Splicing Increases Calcium Channel Current Density in the Pain Pathway

Experimental Procedures

Whole Tissue RT-PCR

Total RNA was isolated from various parts of the rat nervous system with Trizol reagent (Invitrogen, Carlsbad, Calif.). 2 μg of total RNA from each tissue was reverse transcribed with Superscript II (Invitrogen). 10 ng of the first strand cDNA mixture was analyzed using the Advantage 2 PCR system (BD Biosciences). PCR products were cloned and sequenced to confirm their molecular identity. RT-PCR primers for e37a were: rat37a-up, 5′gctgcgtgttgccggattcattat (SEQ ID NO:1); rat37a-dw, 5′ttcattcgaaccaggcgcttgtag (SEQ ID NO:2) (122 bp) and for e37b: rat37b-up, 5′ctgaatacgacccagctgcgtgtg (SEQ ID NO:3); rat37b-dw,5′ccaggcgcttgtatgcaactcgag (SEQ ID NO:4) (126 bp). PCR was performed using a Robocycler (Stratagene) programmed as follows: 1 cycle of 94° C. for 1 min; followed by 30 cycles of 94° C. for 30 sec, 59° C. for 30 sec, and 72° C. for 30 sec; followed by a final 7 min extension at 72° C. Control amplifications were performed with cDNAs to ensure the primers were specific for e37a and e37b. RT-PCR products were run on 3% agarose gels pre-stained with ethidium bromide and imaged on a digital analysis system (Alpha Innotech, San Leandro, Calif.).

Cell Isolation

Dorsal root ganglia were harvested from all spinal levels of P5 to P8 rats (Sprague Dawley, Charles River, Wilmington, Mass.). Ganglia were dissociated in a solution containing supplemented L-15 media with collagenase (20 mg/ml, Sigma Chemical Co., St. Louis, Mo.) and dispase (96 mg/ml, Sigma). Enzymatic digestion was carried out at 37° C. for 45 min and cells triturated periodically with a fire polished pipette to facilitate dissociation. Neurons were washed twice in 1×PBS (Gibco Invitrogen) and plated on poly-D-lysine (Sigma) coated covers slips in DMEM (Gibco) containing 10% FBS (Gibco) and NGF (1 ng/ml, Sigma). Neurons were maintained at 37° C. with 5% CO₂ and used within 1 to 6 hrs after isolation.

Capsaicin Screening

Viable neurons were identified by the presence of robust voltage-gated sodium and potassium currents during brief depolarizations from a holding potential of −80 mV. Cells were then maintained at a holding potential of −60 mV and challenged with 2 μM capsaicin to identify responsive and non-responsive neurons. Neurons were then washed with capsaicin-free media. For inclusion in the analysis, capsaicin-induced inward currents had to be at least 25 pA and recover after capsaicin removal. Capsaicin induced currents ranged from 25 pA to 4500 pA. Capsaicin-non-responsive neurons were exposed to capsaicin at least twice before the cell was harvested and counted as capsaicin-non-responsive. Neurons were limited to two exposures of capsaicin to avoid desensitization. For capsaicin screening, currents were recorded using 1-2 MΩ microelectrodes filled with an internal solution consisting of 90 mM KCl, 10 mM NaCl, 5 mM MgCl₂ 40 mM HEPES, and 5 mM EGTA adjusted to a pH of 7.4 with KOH. The bath solution contained 140 mM NaCl, 5 mM KCl, 2 mM CaCl₂, 0.5 mM MgCl₂, 10 mM HEPES, and 15 mM dextrose, adjusted to a pH of 7.2 with NaOH. At the conclusion of the recording the entire cell was harvested via the recording pipette by negative pressure and stored in first-strand buffer at −80° C. until needed. Data acquisition was performed using PClamp V8.1 software and the Axopatch 200A (Axon Instruments, Union City, Calif.). Data was filtered at 2 kHz (−3 dB) and sampled at 2 kHz. No leak correction or series resistance compensation was performed. Recordings were performed at room temperature.

Calcium Current Recordings

Acutely dissociated DRG neurons were placed into the recording chamber containing bathing solution (see above). Patch electrodes were fire polished to a resistance of 1-2 MΩ. The internal solution for perforated patch recordings consisted of 135 mM CsCl, 10 mM HEPES, 1 mM EGTA, 1 mM EDTA, 4 mM MgCl₂ and 1.2 mg/ml amphotericin (Sigma), adjusted to a pH of 7.2 with TEA-OH. Micropipettes were pre-filled with amphotericin-free solution and backfilled with internal solution containing amphotericin. Seals were formed in bathing solution. The series resistance decreased over a 20 min period and was considered acceptable when <12 MΩ. The bath solution was then replaced with 135 mM TEA-Cl, 2 mM CaCl₂, 10 mM HEPES, 100 nM TTX (Sigma), adjusted to a pH of 7.2 with TEA-OH. Whole cell calcium currents were recorded under constant bath perfusion. A current-voltage protocol from a holding potential of −80 mV with 50 ms test pulses was then delivered. ω-Ctx GVIA (3 μM; Calbiochem, San Diego, Calif.) was applied directly onto the cell via a pipette using positive pressure. Test pulses were applied every 20 seconds to monitor toxin block which was considered saturated when current amplitudes were constant over a period of 1 minute. ω-Ctx GVIA was completely removed from the bath and a second current-voltage relationship obtained. Finally, the bath was exchanged with Na-containing solution, the cell voltage clamped to −60 mV, and challenged with 2 μM capsaicin. The entire cell was then harvested and stored in first-strand buffer at −80° C. After each application of ω-Ctx GVIA, the recording chamber was soaked in 2% SDS and 10 mM DTT for 15 min to remove any residual ω-Ctx GVIA. Data acquisition was performed using pClamp V8.1 software and the Axopatch 200A (Axon Instruments). For recordings calcium currents, data was filtered at 2 kHz (−3 dB) and sampled at 20 kHz. The series resistance was compensated by 70-90% with a 10 μs lag. Leak correction was performed with a P/−4 protocol. All recordings were obtained at room temperature.

The Low Threshold Component in Toxin-Subtracted Currents from Capsaicin-Nonresponsive Cells

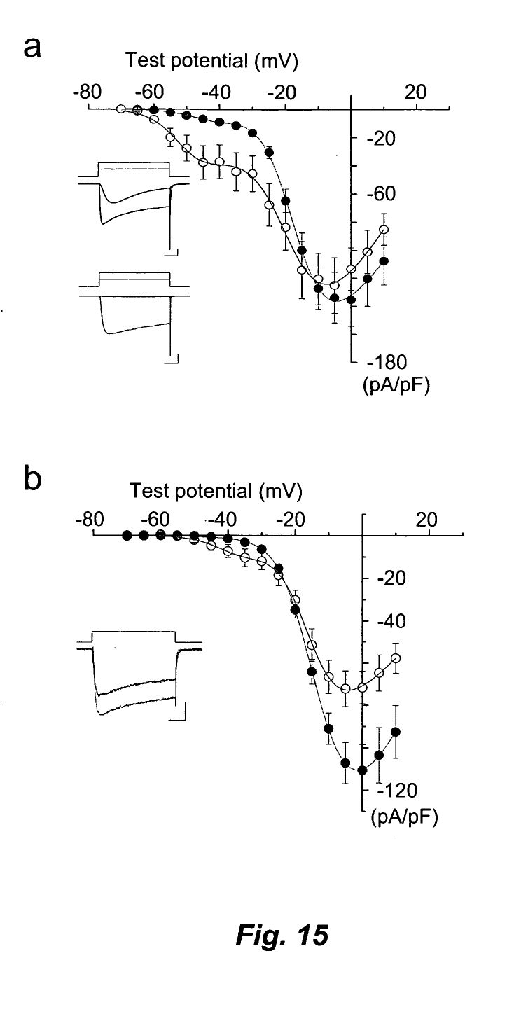

A low threshold calcium current was prominent in capsaicin-non-responsive cells (FIG. 15a). This current deactivated with slow kinetics, typical of T-type currents but it was also labile. Current amplitudes declined to 52±2% of initial values over a 5 minute period (measured at −40 mV, n=3) compared to a decline to 89±1% for the high threshold current (at −10 mV, n=3). Relatively rapid run-down of the low threshold current likely explains the presence of a low threshold current showing up in the ω-Ctx GVIA-sensitive current voltage relationship. It is also possible that other low threshold calcium currents contribute to this component of the current voltage-relationship. For example, Ca_{V}1.3 L-type currents that activate at relatively negative voltages (Lipscombe, 2002). There are reports that the Ca_{V}1.3 L-type current is inhibited by high concentrations of ω-Ctx GVIA (Williams et al., 1992b), however, our studies of neuronal Ca_{V}1.3 L-type channels suggest that they are not ω-Ctx GVIA-sensitive (Xu and Lipscombe, 2001).

Single Cell RT-PCR

After harvesting the entire neuron into the recording pipette via negative pressure, the tip of the pipette and its contents (˜2 μl) were broken into a thin-walled PCR tube containing water and first-strand buffer. cDNA was synthesized from single cells using Superscript II (Invitrogen) and used as template for subsequent PCR reactions with the Advantage2 PCR system (BD Biosciences Clontech, Palo Alto, Calif.). One round of PCR was performed using a Robocyler (Stratagene, La Jolla, Calif.) on single cell cDNA. For all sets of primers the PCR reaction conditions were 1 cycle of 94° C. for 3 min; 35 cycles of 94° C. for 1 min, 59° C. for 1.5 min, 72° C. for 1.5 min, and a final 7 min extension at 72° C. Products were then run out on 2% standard agarose or 4% Metaphor agarose gels. Band intensities were directly quantified from gels using a digital analysis system (Alpha Innotech). PCR products from single cells were subcloned and sequenced to confirm their identity. Negative controls lacking cDNA were performed and processed in parallel with single cell RT-PCR reactions (1 negative control for every 6 cells). The negative controls included recording pipettes used for whole cell recording. To prevent contamination all PCR reactions were performed in a separate room not previously exposed to Ca_{V}2.2 clones. Internal solutions were RNAse free. RT-PCR primers were located in different exons to avoid amplification of genomic DNA. RT-PCR reagents and enzymes were always divided into aliquots to screen single cells in groups of ˜50 neurons with identical stocks of RT-PCR reagents and enzymes. This reduced variability. The PCR primer sequences and their predicted PCR product sizes were as follows:

| | |
|---|---|
| r18a-up, | |
| 5′cgcaggttctggagccttagct; | (SEQ ID NO: 5) |
| r18a-dw, | |
| 5′ggccattgctgtggacaacctt. | (SEQ ID NO: 6) |
| (227 and 290 bp for .18a and +18a) | |
| r24a-up, | |
| 5′cattctggacttcattgttgtcagtgga; | (SEQ ID NO: 7) |
| r24a-dw, | |
| 5′tcgcaggactctcagagacttgatggta. | (SEQ ID NO: 8) |
| (114 and 126 bp for .24a and +24a) | |
| r31a-up, | |
| 5′cagagatgcctggaacgtctttgac; | (SEQ ID NO: 9) |
| r31a-dw, | |
| 5′ataacaagatgcggatggtgtagcc. | (SEQ ID NO: 10) |
| (169 and 175 bp for .31a and +31a) | |
| r37a-up, | |
| 5′agctgcgtgttgccggattcattataagga; | (SEQ ID NO: 11) |
| r37a-dw, | |
| 5′gcgcttgtaggccaacctacgagggcagtt. | (SEQ ID NO: 12) |
| (108 bp) | |
| r37b-up, | |
| 5′agctgcgtgtgggcgcatcagttacaatga; | (SEQ ID NO: 13) |
| r37b-dw, | |
| 5′gcgcttgtatgcaactcgagccgggcattt. | (SEQ ID NO: 14) |
| (108 bp) | |
| rVR1-up, | |
| 5′tcaattcccacacacctcccagttcc; | (SEQ ID NO: 15) |
| rVR1-dw, | |
| 5′tttgggtggtctgcttagggaaccag. | (SEQ ID NO: 16) |
| (125 bp) | |
| rNaV1.8-up, | |
| 5′gcgctggttgtaagggtcagattg; | (SEQ ID NO: 17) |
| rNaV1.8-dw, | |
| 5′agccaggcaacaattgcagaaatc. | (SEQ ID NO: 18) |
| (245 bp) | |
| rGAPDH-up, | |
| 5′cagcaccagcatcaccccattt; | (SEQ ID NO: 19) |
| rGAPDH-dw, | |
| 5′caagatggtgaaggtcggtgtgaa. | (SEQ ID NO: 20) |
| (274 bp) | |

| | |
|---|---|
| Competitive template for e37a, | |
| 5′agctgcgtgttgccggattcattataaggat | (SEQ ID NO: 21) |
| aagcgtgctggactctctcgacgcaaatcgagg | |
| cccaattggcaaatacagttgcagaacaggagg | |
| agccacacaactgccctcgtaggttggcctaca | |
| agcgc; (135 bp) | |
| for e37b, | |
| 5′agctgcgtgtgggcgcatcagttgcaatgat | (SEQ ID NO: 22) |
| aagcgtgctggactctctcgacgcaaatcgagg | |
| cccaattggcaaat acagttgcagaacaggag | |
| gagccacacaaaagcccggctcgagttgcatac | |
| aagcgc. (135 bp) | |

Oocyte Expression and Physiology

Complementary RNAs (cRNAs) were synthesized in vitro using the Ambion mMessage machine RNA transcription kit using T-7 primer (Ambion, Austin, Tex.). 1 μl of each Ca_{V}2.2 cRNA preparation was run out on a denaturing formaldehyde gel along with cRNA standards. Band intensities were directly quantified using a digital analysis system (Quantity-One, Bio-Rad, Hercules, Calif.) to ensure equal amounts of Ca_{V}2.2e[37a] and Ca_{V}2.2e[37b] cRNAs. Full-length cRNAs were mixed in equimolar ratios, 5 ng of Ca_{V}2.2, 1.6 ng of Ca_{V}β3, and 2.6 ng of Ca_{V}α₂δ in a total volume of 46 nl, and injected into individual defolliculated oocytes. Four separate rounds of oocyte injections were performed. N-type calcium currents were recorded 4-6 days after injection by standard two microelectrode voltage clamp technique (Warner amplifier; OC-725b) and the data collected using pClamp6 software (Axon Instruments, Union City, Calif.). Microelectrodes were filled with 3M KCl and the resistances of the current and voltage electrodes were 0.3-1.5 MΩ. Data were filtered at 0.5 kHz and sampled at 10 kHz. Currents were recorded in a reduced chloride bathing solution containing: 5 mM Ba(OH)₂, 5 mM HEPES, 85 mM TEA-OH, and 2 mM KOH, pH adjusted to 7.4 with methanesulfonic acid. N-type currents ranged between 0.1 μA and 0.6 μA. Leak and capacitive currents were subtracted online using a P/−4 protocol (Pclamp, V6.0; Axon Instruments).

Results

Multiple Ca_{V}2.2 Splice Isoforms are Expressed in Dorsal Root Ganglia

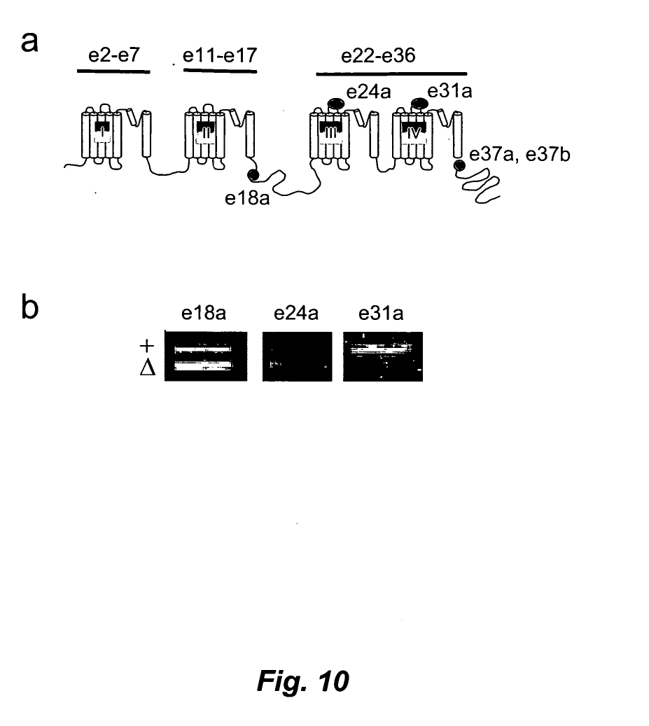

The Ca_{V}2.2 gene is subject to alternative splicing and dorsal root ganglia express multiple splice isoforms (FIG. 10a, b). Cassette exons e18a, e24a, and e31a are either present or not in the final mRNAs. Their expression patterns have been reasonably well characterized at the whole tissue level. The majority of Ca_{V}2.2 mRNA in dorsal root ganglia contains e31a, consistent with preferential expression of this exon in peripheral ganglia. Splice isoforms of Ca_{V}2.2 both containing and lacking e18a and e24a are also present (FIG. 10b). The splicing patterns of e18a, e24a, and e31a of Ca_{V}2.2 at the whole tissue level is consistent with previous analyses by RT-PCR and ribonuclease protection in rat DRG (Lin et al., 1997; Lin et al., 1999; Pan and Lipscombe, 2000) and for equivalent exons 24a and 31a in chick DRG (Lu and Dunlap, 1999). These analyses of whole tissue, however, do not address whether the pattern of alternative splicing varies among individual neurons. Sensitivity to capsaicin is commonly used as a functional marker to identify a subtype of nociceptive neuron (Caterina and Julius, 2001; Caterina et al., 1997; Tominaga et al., 1998). We therefore used whole-cell recording to separate neurons into two groups based on capsaicin-responsiveness, and then analyzed each site of alternative splicing in the same neurons by single cell RT-PCR.

Screening Neurons of the Dorsal Root Ganglia by Capsaicin-Responsiveness

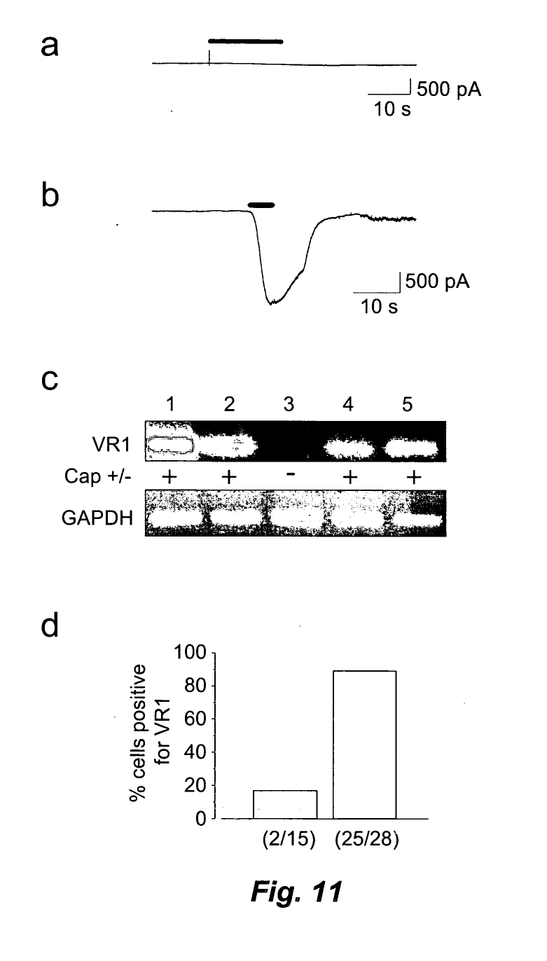

Neurons of the dorsal root ganglia were voltage-clamped at −80 mV, and brief depolarizations applied to establish the presence of robust voltage-gated potassium and sodium currents characteristic of viable neurons. Subsequently, the membrane potential was voltage-clamped to −60 mV and neurons exposed to 2 μM capsaicin (FIG. 1a, b; n=128 capsaicin-responsive neurons and n=141 non-responsive neurons). Capsaicin exerts its effects via activation of the vanilloid receptor (VR1). Consistent with this, we detected VR1 mRNA in 89% of capsaicin-responsive cells (25 of 28) whereas only 13% of non-responsive cells contained VR1 mRNA (2 of 15) (FIG. 11c, d).

Individual DRG Neurons Express Multiple Ca_{V}2.2 Splice Isoforms

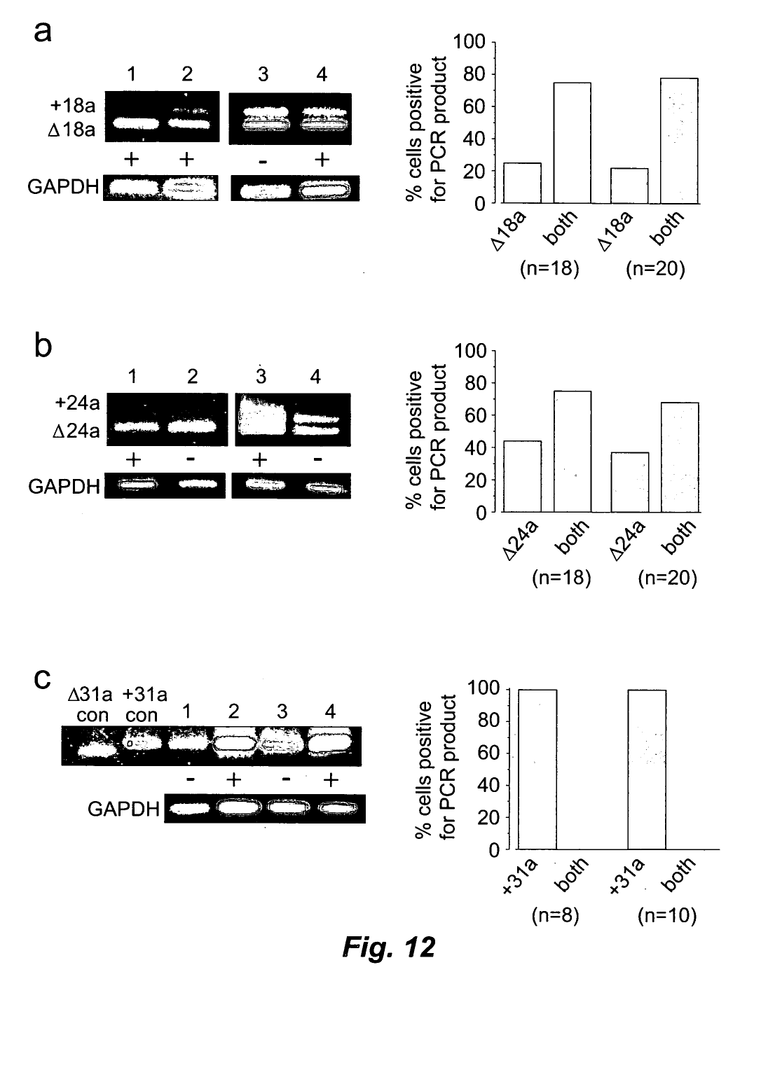

We analyzed e18a, e24a, and e31a at the single cell level and demonstrated that the pattern of alternative splicing varied among individual neurons. Some cells expressed both isoforms of a particular splice site, while other cells expressed one form (FIG. 12). For example, all neurons expressed Ca_{V}2.2 mRNAs that lacked e18a (FIG. 12a, n=38 cells). While only a sub-set also expressed Ca_{V}2.2 mRNAs containing e18a. We observed a pattern of alternative splicing similar to this for the e24a site (FIG. 12b, n=38). However, there was no correlation between the inclusion or exclusion of a particular exon and capsaicin-responsiveness (FIG. 12a, b). We detected e31a in all neurons analyzed regardless of capsaicin responsiveness. Ca_{V}2.2 mRNAs lacking e31a were not detected (FIG. 12c; n=18). This reflects the splicing pattern of e31a observed at the whole tissue level and is consistent with the proposal that the vast majority of Ca_{V}2.2 mRNAs expressed in peripheral ganglia, including DRG, contain e31a (Lin et al., 1997; Lin et al., 1999; Lu and Dunlap, 1999).

Exon 37a is Only Expressed in Dorsal Root Ganglia

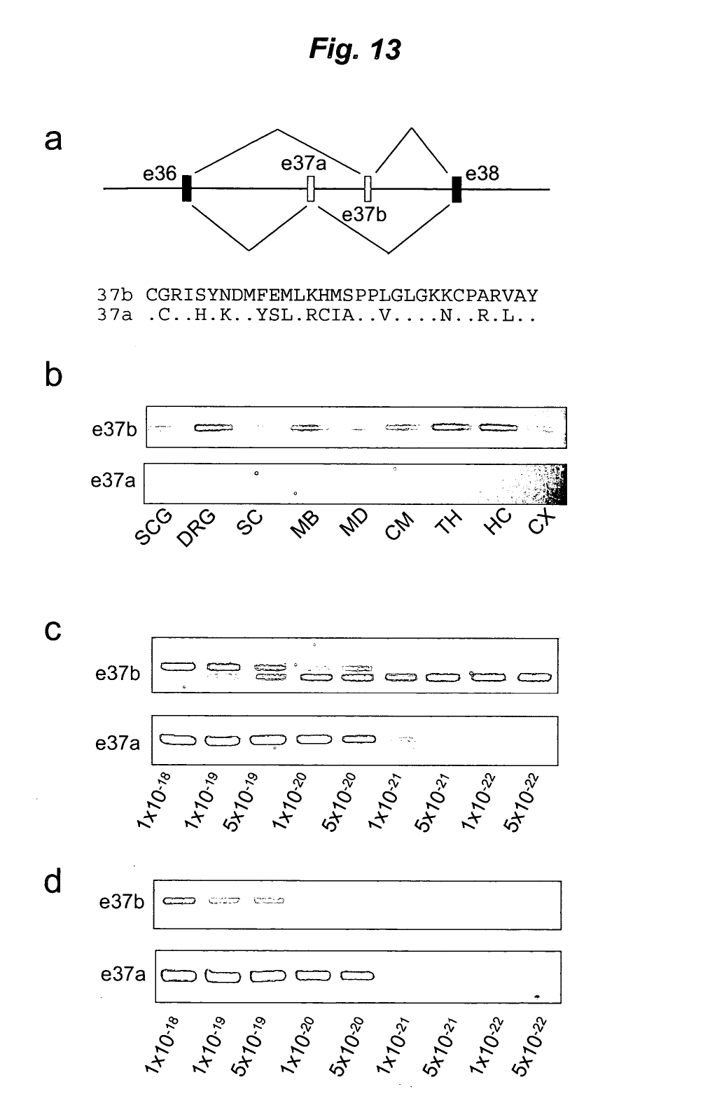

We examined a fourth site of alternative splicing, located in the C-terminus, that had been identified based on analysis of human genomic sequence (Lipscombe, 2003; Lipscombe et al., 2002; Soong et al., 2002). The presence of tandem exons, e37a and e37b in genomic sequence, identical in length (97 nucleotides) and differing by only 14 amino acids is consistent with a mechanism of mutually exclusive splicing (FIG. 13a). However, despite evidence for the presence of these two homologous exons of Ca_{V}2.2 in the public genome sequence of human, rat and mouse, all Ca_{V}2.2 mRNAs analyzed to date contain e37b. No mammalian Ca_{V}2.2 mRNA containing e37a has been reported. We designed two sets of exon-specific PCR primers, because e37a and e37b are indistinguishable on the basis of size. We analyzed their expression patterns in rat, initially at the whole tissue level. As expected, Ca_{V}2.2e[37b] mRNAs were expressed throughout the central nervous system and in both sympathetic and dorsal root ganglia. In sharp contrast, we found expression of e37a highly restricted and only present at significant levels in dorsal root ganglia (FIG. 13b). Our results explain why Ca_{V}2.2e[37a] cDNAs were not isolated in earlier cloning efforts because tissue from dorsal root ganglia was not used (Cahill et al., 2000; Coppola et al., 1994; Dubel et al., 1992; Fujita et al., 1993; Lin et al., 1997; Williams et al., 1992a). Our use of exon-specific primers, however, does not provide information on the relative levels of Ca_{V}2.2e[37a] and Ca_{V}2.2e[37b] mRNAs. The high efficiency of PCR could result in significant product even if starting levels of Ca_{V}2.2e[37a] mRNAs are low in number. We therefore employed a competitive RT-PCR protocol and established that Ca_{V}2.2e[37a] mRNAs are present in whole DRG of P5 and adult animals at levels only 10-fold lower compared to Ca_{V}2.2e[37b] (FIGS. 13c and 13d). The Ca_{V}2.2e[37a] splice isoform therefore represents a significant fraction of the total Ca_{V}2.2 mRNA pool in dorsal root ganglia. We next tested the possibility that e37a expression is regulated at the single cell level and perhaps linked to functionally distinct sub-types of sensory neurons.

Preferential Expression of Exon e37a in a Subset of Capsaicin-Responsive Neurons

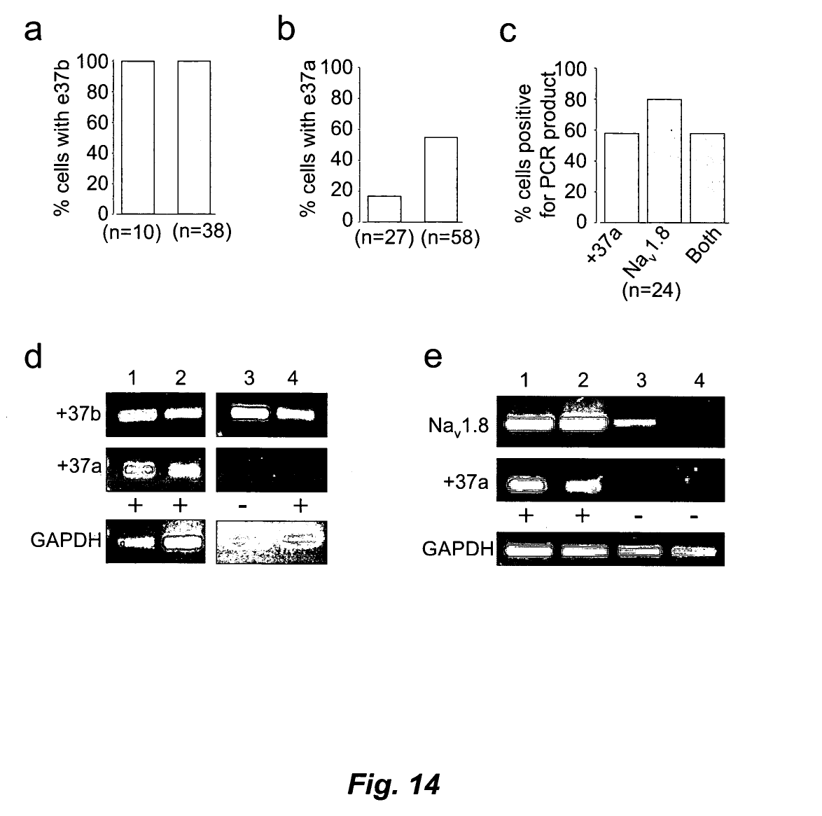

We separated neurons of the dorsal root ganglia on the basis of capsaicin-responsiveness and analyzed each cell by RT-PCR using e37a and e37b-specific primers. All neurons tested with e37b-specific primers contained e37b (n=48; FIG. 14a, 14d). This is consistent with the ubiquitous expression pattern of this splice form of Ca_{V}2.2 (FIG. 13b) and also serves as an important control establishing that Ca_{V}2.2 mRNA is expressed in every neuron. By contrast, only a sub-set of neurons expressed e37a. Most significantly, the e37a splice isoform was expressed preferentially in capsaicinresponsive neurons (55% of capsaicin-responsive compared to 17% of non-responsive neurons; FIG. 14b, 14d). Capsaicin-sensitivity is often used as a marker for nociceptors but it only identifies a sub-set of these cells. We therefore assayed for the presence of the TTX-resistant sodium channel, Na_{V}1.8, that is restricted to nociceptors (Akopian et al., 1996; Dib-Hajj et al., 1998). Using Na_{V}1.8-specific primers we found that 80% of capsaicin-responsive neurons analyzed express Na_{V}1.8 mRNA (19 of 24; FIG. 14c, 14e). Most significantly, all neurons that expressed e37a also expressed Na_{V}1.8 (14 of 14; FIG. 14c, 14e) establishing a strong link between a second molecular marker of nociceptors and the presence of Ca_{V}2.2 mRNA containing e37a. Importantly, we next assessed the functional consequences of cell-specific expression of Ca_{V}2.2e[37a] on the N-type current.

Calcium Currents in Capsaicin-Responsive and Non-Responsive Neurons

We used the perforated, whole-cell patch recording method to record calcium currents from neurons of the dorsal root ganglia (Horn and Marty, 1988). 2 mM calcium was the charge carrier. Capsaicin-responsive neurons were significantly smaller in size on average (18±3 pF), compared to non-responsive neurons (27 pF±3) (p<0.001) as reported by others (Cardenas et al., 1995). Initially, the most obvious difference among cells when we separated them on the basis of capsaicin-sensitivity was the presence of a significantly larger low threshold Ca current in non-responsive neurons. This low threshold component is prominent in exemplar current traces and in the average current voltage-relationships from non-responsive neurons (FIG. 15a). In this regard, our findings are consistent with the reports of others (Blair and Bean, 2002; Carbone and Lux, 1984; Cardenas et al., 1995; Schroeder et al., 1990; Scroggs and Fox, 1992a). The high threshold component of the whole cell calcium channel current originates from the activity of additional classes of calcium channel including N-type (Regan et al., 1991; Scroggs and Fox, 1992a). On first inspection, the multi-component, high voltage-activated current appeared similar between capsaicin-responsive and non-responsive neurons (FIG. 15a, activation mid-points for the high voltage-activated currents were close to −15 mV). For this study, we were specifically interested in the N-type current and isolated it from other voltage-activated calcium currents by toxin-subtraction using ω-conotoxin GVIA (ω-Ctx GVIA) (Regan et al., 1991; Scroggs and Fox, 1992b).

N-type Currents in Capsaicin-Responsive and Non-Responsive Neurons

Consistent with other reports, the ω-Ctx GVIA-sensitive, N-type current was the major component of the total Ca current in capsaicin-responsive and non-responsive neurons (˜70%; FIG. 15b) (Regan et al., 1991; Scroggs and Fox, 1992a). The N-type current was well isolated in capsaicin-responsive neurons although in non-responsive neurons a small low-threshold current was still present in toxin-subtracted currents (FIG. 15b). The low threshold current runs down relatively rapidly even in the absence of toxin, and this is the most likely explanation for its presence in toxin-subtracted current-voltage relationships of non-responsive neurons (see Methods section for further details). Most importantly, after isolating the N-type current we revealed significant differences in peak N-type channel current densities between capsaicin-responsive 111±12 pA/pF (n=20) and non-responsive neurons 72±8 pA/pF (n=9) (FIG. 15b; p<0.05). Although significantly larger, the biophysical properties of N-type currents in capsaicin-responsive neurons were not distinguishable from non-responsive neurons (V_{1/2} and k values for non-responsive neurons were: −16±2 mV and 5.4±0.6, n=9, compared to −15±1 mV and 5.2±0.3 for responsive neurons, n=20; FIG. 15b). Our data suggests that capsaicin-responsiveness is linked to a significantly greater number of functional N-type channels.

Exon 37a is Linked to Larger N-type Current Density in Capsaicin-Responsive Neurons

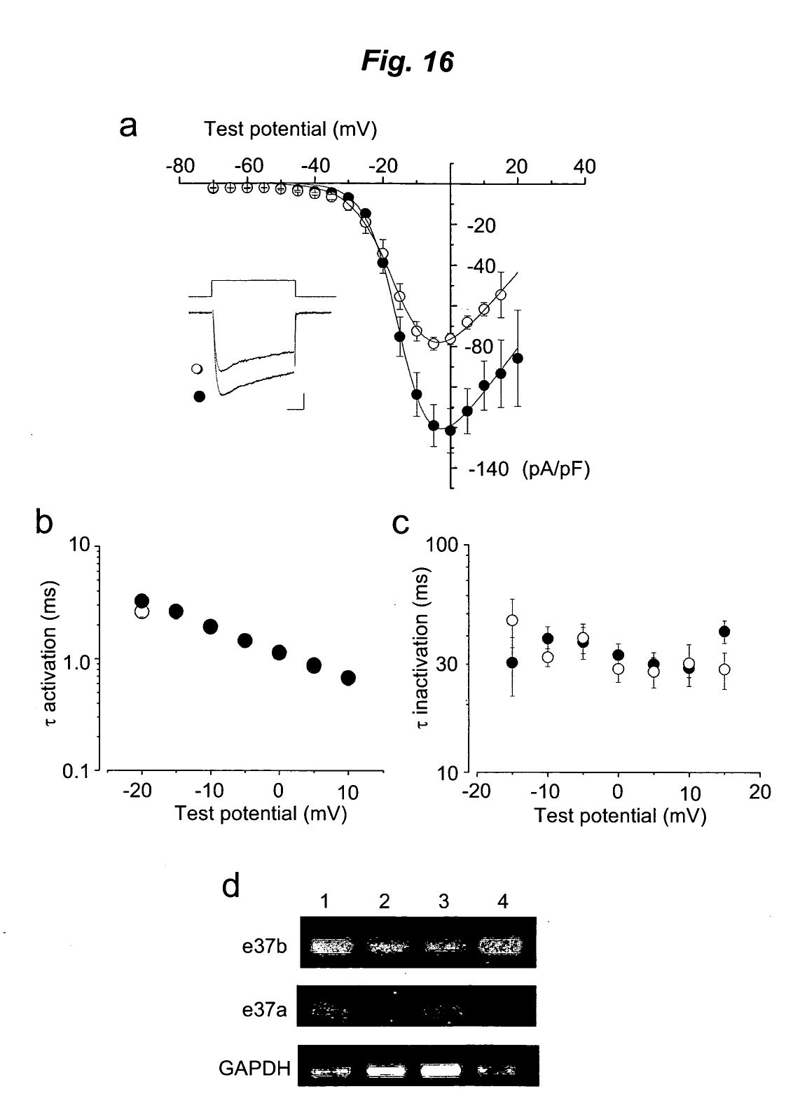

Having demonstrated preferential expression of a unique splice isoform of Ca_{V}2.2 in a sub-set of capsaicin-responsive neurons (FIG. 14b-d), we tested the hypothesis that the presence of Ca_{V}2.2e[37a] was linked to larger N-type current densities. We selected only capsaicin-responsive neurons, analyzed the N-type current, harvested these same cells, and performed RT-PCR to assay for the presence of each of Ca_{V}2.2e[37a], Ca_{V}2.2e[37b], and GAPDH. We performed this complete series of manipulations on 16 individual capsaicin-responsive neurons. These were subsequently separated into two pools, those that expressed Ca_{V}2.2e[37a] mRNA and those that did not. FIG. 16 summarizes our results and reveals a significant difference in N-type current densities between cells that contain and lack Ca_{V}2.2e[37a]. Capsaicin-responsive neurons that contained and lacked exon 37a were not distinguishable based on cell size, consistent with them representing a relatively homogenous population of nociceptors (20±3 pF, n=8, and 18±1 pF, n=8, for e37a-containing and lacking, respectively). The presence of the Ca_{V}2.2e[37a] splice isoform was, however, associated with 1.6-fold larger N-type currents (peak current densities were 122±11 pA/pF, n=8, in cells containing e37a compared to 76±3 pA/pF, n=8, in cells lacking e37a; p<0.05; FIG. 16). We also compared the basic biophysical properties of the N-type currents. The voltage-dependence of N-type channel activation, as well as the kinetics of activation and inactivation is not significantly different in cells containing and lacking exon 37a, despite their very different current densities (FIG. 16a-c).

Exon 37a Induces Larger N-type Currents in a Non-Neuronal Expression System

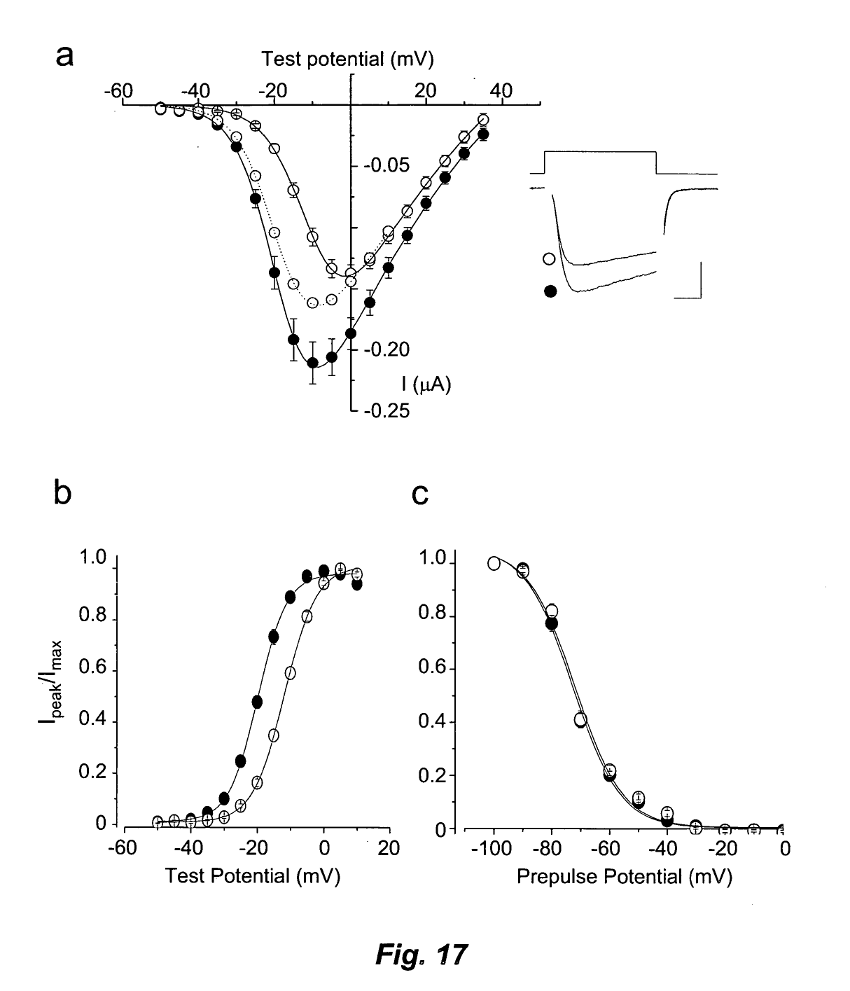

To establish a direct link between expression of the Ca_{V}2.2e[37a] splice isoform and larger N-type channel current amplitudes, and to eliminate the potential contribution of other factors, we compared Ca_{V}2.2e[37a] and Ca_{V}2.2e[37b] clones expressed in Xenopus oocytes under identical conditions. The resultant N-type channel currents were compared in detail and our analysis summarized in FIG. 17. N-type channel currents in oocytes injected with Ca_{V}2.2e[37a] cRNA were consistently and significantly larger than those in oocytes injected with the same amount of Ca_{V}2.2e[37b] cRNA (1.6-fold, p<0.05; FIG. 17). Currents were recorded under identical conditions and the same complement of auxiliary subunits co-expressed. These differences between isoforms were observed over a three-day period (see legend to FIG. 17). We also compared the biophysical properties of the two splice isoforms. The voltage-dependence and kinetics of N-type channel activation were different between Ca_{V}2.2e[37a] and Ca_{V}2.2e[37b] channels. Ca_{V}2.2e[37a] channels activated at voltages 8 mV more hyperpolarized and with slightly faster kinetics at the same voltages, compared to Ca_{V}2.2e[37b] (FIG. 17b). To estimate the effect of left shifting the Ca_{V}2.2 channel activation on current amplitude, we plotted the predicted current-voltage relationship for Ca_{V}2.2e[37b] using the activation curve for Ca_{V}2.2e[37a] (FIG. 17a, dashed line). An 8 mV shift in activation results in a small increase in peak current because of the greater driving force on ions at hyperpolarized voltages, but this is insufficient to account for the 1.6-fold difference in peak current amplitudes between Ca_{V}2.2e[37a] and Ca_{V}2.2e[37b]. We also demonstrated that the voltage-dependence and kinetics of channel inactivation were indistinguishable between splice isoforms (FIG. 17c and see legend). These latter results exclude the possibility that differences in sensitivity to steady-state inactivation between splice isoforms might underlie their different current amplitudes.

Discussion

N-type calcium channels are critical for the transduction of pain in nociceptive neurons of dorsal root ganglia. Here we identify a novel splice form of the N-type Ca_{V}2.2 subunit restricted to dorsal root ganglia, and within this tissue, preferentially expressed in a subset of nociceptive neurons. mRNA levels of this Ca_{V}2.2 splice isoform are substantial and e37a-containing Ca_{V}2.2 mRNA strongly correlates with significantly larger N-type calcium current densities. Cell-specific alternative splicing at the e37a/e37b locus of Ca_{V}2.2 provides a mechanism for increasing N-type current density down-stream of gene transcription.

Functional Implications

The crucial role for the N-type channel in nociception is supported by several lines of evidence including the localization of N-type channels at primary synapses of nociceptive neurons in the dorsal horn laminae I/II (Kerr et al., 1988), dominance in controlling neurotransmitter release from presynaptic terminals of nociceptive neurons (Holz et al., 1988; Maggi et al., 1990), impaired nociception in mice lacking Ca_{V}2.2 (Hatakeyama et al., 2001; Kim et al., 2001; Saegusa et al., 2001), and powerful analgesic properties of spinally-administrated N-type channel blockers in a variety of animal pain models (Bowersox et al., 1996; Bowersox and Luther, 1998; Bowersox et al., 1995; Matthews and Dickenson, 2001; Vanegas and Schaible, 2000). The N-type channel is a target for modulation by many neuromodulators and second messenger signaling pathways. Most neuromodulators, including morphine, couple to the N-type channel to inhibit its activity and down-regulate transmitter release (Ikeda and Dunlap, 1999). Here, we describe a mechanism for increasing the activity of the N-type current in a sub-set of nociceptive neurons that has the potential to increase the coupling efficiency between depolarization and transmitter release, thereby modifying the transmission of noxious stimuli. A characteristic feature of nociceptive neurons is their unusually wide action potentials that have a signature shoulder phase (Blair and Bean, 2002; Djouhri et al., 1998; Dunlap and Fischbach, 1978; Harper and Lawson, 1985; Heyman and Rang, 1985; Ritter and Mendell, 1992). High voltage-activated calcium channels, predominantly N-type, together with TTX-resistant sodium channels are the main contributors to the plateau phase (Baccei and Kocsis, 2000; Blair and Bean, 2002; Renganathan et al., 2001; Scroggs and Fox, 1992b). Cell-specific alternative splicing of e37a in nociceptive neurons also represents a potential mechanism for shaping action potential duration by increasing N-type current density. Although other ion channels clearly remain important, it would be interesting to determine if a correlation exists between levels of e37a and action potential half-widths in single cells. Inclusion of e37a during the processing of Ca_{V}2.2 RNA is cell-specific, raising the possibility that this splicing event might be regulated by specific stimuli or level of neuronal activity. For example, alternative splicing of the STREX exon in the calcium-activated potassium channel, slo, is regulated by stress hormones and membrane depolarization (Xie and Black, 2001; Xie and McCobb, 1998). mRNA and protein levels of the calcium channel Ca_{V}α2δ subunit protein have been reported to be elevated in dorsal root ganglia in a rat injury model (Luo et al., 2001). The Ca_{V}α2δ subunit modifies calcium channel properties and increases channel surface expression (Felix, 1999; Witcher et al., 1993). Although total levels of Ca_{V}2.2 mRNA were reportedly unchanged in the same study (Luo et al., 2001) the splicing pattern at the e37a/e37b locus was not analyzed. Our data suggests that a shift in the splicing pattern from e37bcontaining to e37a-containing Ca_{V}2.2 mRNA could provide a powerful and relatively rapid mechanism, independent of gene transcription, for increasing N-type current density in the pain pathway.

Mechanism of Action of Exon 37a

Our experiments in Xenopus oocytes with cloned Ca_{V}2.2 channels demonstrate a direct link between the presence of exon 37a and significantly larger N-type currents. In this respect our studies of cloned channels parallel nicely those of native N-type currents in capsaicin-responsive neurons. Although other factors need to be ruled out, such as differences in single channel conductance, our results are most consistent with the hypothesis that Ca_{V}2.2e[37a] splice isoforms produce a greater number of functional N-type channels compared to Ca_{V}2.2e[37b]. Our analysis of Ca_{V}2.2e[37a] and Ca_{V}2.2e[37b] currents recorded from oocytes did, however, also reveal differences in the voltage-dependence and kinetics of N-type channel activation. By contrast, the biophysical properties of native N-type currents isolated by conotoxin-subtraction were not significantly different in capsaicin-responsive cells separated based on the presence and absence of Ca_{V}2.2e[37a] (FIG. 16a-c). It is possible that the biophysical properties that differ between splice isoforms in the oocyte expression system are present in native currents, but masked in our analysis by a combination of the toxin-subtraction method and the presence of other splice isoforms with different properties (FIG. 10) (Lipscombe et al., 2002). While insufficient alone to account for the different current amplitudes, if present in neurons, such biophysical differences between splice isoforms would combine with the increased effectiveness of Ca_{V}2.2e[37a] mRNA, to induce larger N-type currents and increase calcium entry through the N-type pathway. Our oocyte experiments demonstrate that the mechanisms underling the different N-type current amplitudes induced by splice isoforms are not unique to neurons, and further, that the information necessary to mediate these differences are contained in the 14 amino acids that are different between Ca_{V}2.2e[37a] and Ca_{V}2.2e[37b] (FIG. 13a). The 37a/37b splice site is located in the C-terminus of Ca_{V}2.2, a region implicated in the trafficking and targeting of calcium channels (Gao et al., 2000; Maximov and Bezprozvanny, 2002; Maximov et al., 1999). It is possible that the Ca_{V}2.2e[37a] isoform is more efficiently targeted to the plasma membrane, although other mechanisms including differences in protein stability cannot be ruled out. The C-terminus of Ca_{V}2.2 constitutes ˜25% of the channel protein and is encoded by exons 36-46 (FIG. 10a). It contains several protein binding sites including those that bind G-proteins, Mintl, CASK, and RIM binding proteins (Hibino et al., 2002; Maximov et al., 1999; Simen et al., 2001). However, these sites fall distal to the e37a/e37b locus. Further, all binding studies using recombinant Ca_{V}2.2 protein to date have been performed with the e37b-containing splice form. A comparison of amino acid sequences suggests that e37a modifies a putative EF-hand motif present in e37b. However, so far we have not observed calcium-dependent differences between splice forms transiently expressed in a mammalian cell line even with low internal calcium buffering. Exons homologous to e37a and e37b are present in the closely related Ca_{V}2.1 gene of the P/Qtype channel and are also predicted to modify a putative EF-hand motif (Soong et al., 2002). The functional significance of splicing at the e37a/e37b locus in Ca_{V}2.1 is not known.

Potential Therapeutic Implications

N-type channel antagonists that target sequences specific to e37a of Ca_{V}2.2, could offer significant benefits over current N-type blockers such as SNX-111 that have been used with limited success as analgesics because of undesirable side effects. These include inhibition of sympathetic tone, disrupted motor coordination, and dizziness and blurred vision (Bowersox et al., 1996; Brose et al., 1997; Chaplan et al., 1994; Vanegas and Schaible, 2000). Selective inhibition of the e37a splice form of Ca_{V}2.2 represents a potential strategy for targeting N-type currents in a subset of nociceptive neurons while leaving basal transmission mediated by the N-type channel unaffected. It will now be important to determine the sub-cellular distribution of these splice forms of Ca_{V}2.2 in nociceptive neurons using exon-specific antibodies. Other splice forms of the Ca_{V}2.2 subunit arising from alternative splicing at a different locus in the C-terminus have been reported that are differentially targeted in the cell (Maximov and Bezprozvanny, 2002). Cell-specific alternative splicing of other ion channels exists (Black, 1998; Jones et al., 1999; Navaratnam et al., 1997; Rosenblatt et al., 1997) and offers a powerful means to fine-tune the properties of channel activity down-stream of transcription. We demonstrate cell-specific alternative splicing of the N-type Ca channel in a subset of nociceptive neurons and suggest that this offers a mechanism for increasing the efficiency of coupling between membrane depolarization and calcium entry in the pain pathway.

### References

Akopian, A. N., Sivilotti, L., and Wood, J. N. (1996). A tetrodotoxin-resistant voltage-gated sodium channel expressed by sensory neurons. Nature 379, 257-262.

Baccei, M. L., and Kocsis, J. D. (2000). Voltage-gated calcium currents in axotomized adult rat cutaneous afferent neurons. J Neurophysiol 83, 2227-2238.

Black, D. L. (1998). Splicing in the inner ear: a familiar tune, but what are the instruments? Neuron 20, 165-168.

Blair, N. T., and Bean, B. P. (2002). Roles of tetrodotoxin (TTX)-sensitive Na+ current, TTX-resistant Na+ current, and Ca2+ current in the action potentials of nociceptive sensory neurons. J Neurosci 22, 10277-10290.

Bowersox, S. S., Gadbois, T., Singh, T., Pettus, M., Wang, Y. X., and Luther, R. R. (1996). Selective N-type neuronal voltage-sensitive calcium channel blocker, SNX-111, produces spinal antinociception in rat models of acute, persistent and neuropathic pain. J Pharmacol Exp Ther 279, 1243-1249.

Bowersox, S. S., and Luther, R. (1998). Pharmacotherapeutic potential of omegaconotoxin MVIIA (SNX-111), an N-type neuronal calcium channel blocker found in the venom of Conus magus. Toxicon 36, 1651-1658.

Bowersox, S. S., Miljanich, G. P., Sugiura, Y., Li, C., Nadasdi, L., Hoffman, B. B., Ramachandran, J., and Ko, C. P. (1995). Differential blockade of voltage-sensitive calcium channels at the mouse neuromuscular junction by novel omega-conopeptides and omega-agatoxin-IVA. J Pharmacol Exp Ther 273, 248-256.

Brose, W. G., Gutlove, D. P., Luther, R. R., Bowersox, S. S., and McGuire, D. (1997). Use of intrathecal SNX-111, a novel, N-type, voltage-sensitive, calcium channel blocker, in the management of intractable brachial plexus avulsion pain. Clin J Pain 13, 256-259.

Cahill, A. L., Hurley, J. H., and Fox, A. P. (2000). Coexpression of cloned alpha(1B), beta(2a), and alpha(2)/delta subunits produces non-inactivating calcium currents similar to those found in bovine chromaffin cells. J Neurosci 20, 1685-1693.

Carbone, E., and Lux, H. D. (1984). A low voltage-activated, fully inactivating Ca channel in vertebrate sensory neurones. Nature 310, 501-502.

Cardenas, C. G., Del Mar, L. P., and Scroggs, R. S. (1995). Variation in serotonergic inhibition of calcium channel currents in four types of rat sensory neurons differentiated by membrane properties. J Neurophysiol 74, 1870-1879.

Caterina, M. J., and Julius, D. (2001). The vanilloid receptor: a molecular gateway to the pain pathway. Annu Rev Neurosci 24, 487-517.

Caterina, M. J., Schumacher, M. A., Tominaga, M., Rosen, T. A., Levine, J. D., and Julius, D. (1997). The capsaicin receptor: a heat-activated ion channel in the pain pathway. Nature 389, 816-824.

Chaplan, S. R., Pogrel, J. W., and Yaksh, T. L. (1994). Role of voltage-dependent calcium channel subtypes in experimental tactile allodynia. J Pharmacol Exp Ther 269, 1117-1123.

Coppola, T., Waldmann, R., Borsotto, M., Heurteaux, C., Romey, G., Mattei, M. G., and Lazdunski, M. (1994). Molecular cloning of a murine N-type calcium channel alpha 1 subunit. Evidence for isoforms, brain distribution, and chromosomal localization. FEBS Lett 338, 1-5.

Cox, B. (2000). Calcium channel blockers and pain therapy. Curr Rev Pain 4, 488-498.

Dib-Hajj, S. D., Tyrrell, L., Black, J. A., and Waxman, S. G. (1998). NaN, a novel voltagegated Na channel, is expressed preferentially in peripheral sensory neurons and downregulated after axotomy. Proc Natl Acad Sci USA 95, 8963-8968.

Djouhri, L., Bleazard, L., and Lawson, S. N. (1998). Association of somatic action potential shape with sensory receptive properties in guinea-pig dorsal root ganglion neurones. J Physiol 513 (Pt 3), 857-872.

Dubel, S. J., Starr, T. V., Hell, J., Ahlijanian, M. K., Enyeart, J. J., Catterall, W. A., and Snutch, T. P. (1992). Molecular cloning of the alpha-1 subunit of an omega-conotoxinsensitive calcium channel. Proc Natl Acad Sci USA 89, 5058-5062.

Dunlap, K., and Fischbach, G. D. (1978). Neurotransmitters decrease the calcium ocmponent of sensory neurone action potentials. Nature 276, 837-839.

Felix, R. (1999). Voltage-dependent Ca2+ channel alpha2delta auxiliary subunit: structure, function and regulation. Receptors Channels 6, 351-362.

Fujita, Y., Mynlieff, M., Dirksen, R. T., Kim, M. S., Niidome, T., Nakai, J., Friedrich, T., Iwabe, N., Miyata, T., Furuichi, T., and et al. (1993). Primary structure and functional expression of the omega-conotoxin-sensitive N-type calcium channel from rabbit brain. Neuron 10, 585-598.

Gao, T., Bunemann, M., Gerhardstein, B. L., Ma, H., and Hosey, M. M. (2000). Role of the C terminus of the alpha IC (CaV1.2) subunit in membrane targeting of cardiac L type calcium channels. J Biol Chem 275, 25436-25444.

Harper, A. A., and Lawson, S. N. (1985). Conduction velocity is related to morphological cell type in rat dorsal root ganglion neurones. J Physiol 359, 31-46.

Hatakeyama, S., Wakamori, M., Ino, M., Miyamoto, N., Takahashi, E., Yoshinaga, T., Sawvada, K., Imoto, K., Tanaka, I., Yoshizawa, T., et al. (2001). Differential nociceptive responses in mice lacking the alpha(1B) subunit of N-type Ca(2+) channels. Neuroreport 12. 2423-2427.

Heyman, I., and Rang, H. P. (1985). Depolarizing responses to capsaicin in a subpopulation of rat dorsal root ganglion cells. Neurosci Lett 56, 69-75.

Hibino, H., Pironkova, R., Onwumere, O., Vologodskaia, M., Hudspeth, A. J., and Lesage, F. (2002). RIM binding proteins (RBPs) couple Rab3-interacting molecules (RIMs) to voltage-gated Ca(2+) channels. Neuron 34, 411-423.

Holz, G. G. t., Dunlap, K., and Kream, R. M. (1988). Characterization of the electrically evoked release of substance P from dorsal root ganglion neurons: methods and dihydropyridine sensitivity. J Neurosci 8, 463-471.

Horn, R., and Marty, A. (1988). Muscarinic activation of ionic currents measured by a new whole-cell recording method. J Gen Physiol 92, 145-159.

Ikeda, S. R., and Dunlap, K. (1999). Voltage-dependent modulation of N-type calcium channels: role of G protein subunits. Adv Second Messenger Phosphoprotein Res 33, 131-151.

Ino, M., Yoshinaga, T., Wakamori, M., Miyamoto, N., Takahashi, E., Sonoda, J., Kagaya, T., Oki, T., Nagasu, T., Nishizawa, Y., et al. (2001). Functional disorders of the sympathetic nervous system in mice lacking the alpha 1B subunit (Cav 2.2) of N-type calcium channels. Proc Natl Acad Sci USA 98, 5323-5328.

Jones, E. M., Gray-Keller, M., and Fettiplace, R. (1999). The role of Ca2+-activated K+ channel spliced variants in the tonotopic organization of the turtle cochlea. J Physiol 518 (Pt 3), 653-665.

Kerr, I. M., Filloux, F., Olivera, B. M., Jackson, H., and Wamsley, J. K. (1988). Autoradiographic localization of calcium channels with [125I]omega-conotoxin in rat brain. Eur J Pharmacol 146, 181-183.

Kim, C., Jun, K., Lee, T., Kim, S. S., McEnery, M. W., Chin, H., Kim, H. L., Park, J. M., Kim, D. K., Jung, S. J., et al. (2001). Altered nociceptive response in mice deficient in the alpha(1B) subunit of the voltage-dependent calcium channel. Mol Cell Neurosci 18, 235-245.

Lin, Z., Haus, S., Edgerton, J., and Lipscombe, D. (1997). Identification of functionally distinct isoforms of the N-type Ca2+ channel in rat sympathetic ganglia and brain. Neuron 18, 153-166.

Lin, Z., Lin, Y., Schorge, S., Pan, J. Q., Beierlein, M., and Lipscombe, D. (1999). Alternative splicing of a short cassette exon in alpha1B generates functionally distinct Ntype calcium channels in central and peripheral neurons. J Neurosci 19, 5322-5331.

Lipscombe, D. (2002). L-type calcium channels: highs and new lows. Circ Res 90, 933-935.

Lipscombe, D., Castiglioni A. J. (2003). Alternative splicing in voltage-gated calcium channels. In Calcium channel pharmacology, S. I. McDonough, ed. (Kluwer Academic/Plenum).

Lipscombe, D., Pan, J. Q., and Gray, A. C. (2002). Functional diversity in neuronal voltage-gated calcium channels by alternative splicing of Ca(v)alpha1. Mol Neurobiol 26, 21-44.

Lu, Q., and Dunlap, K. (1999). Cloning and functional expression of novel N-type Ca(2+) channel variants. J Biol Chem 274, 34566-34575.

Luo, Z. D., Chaplan, S. R., Higuera, E. S., Sorkin, L. S., Stauderman, K. A., Williams, M. E., and Yaksh, T. L. (2001). Upregulation of dorsal root ganglion (alpha)2(delta) calcium channel subunit and its correlation with allodynia in spinal nerve-injured rats. J Neurosci 21, 1868-1875.

Maggi, C. A., Tramontana, M., Cecconi, R., and Santicioli, P. (1990). Neurochemical evidence for the involvement of N-type calcium channels in transmitter secretion from peripheral endings of sensory nerves in guinea pigs. Neurosci Lett 114, 203-206.

Matthews, E. A., and Dickenson, A. H. (2001). Effects of spinally delivered N- and P-type voltage-dependent calcium channel antagonists on dorsal horn neuronal responses in a rat model of neuropathy. Pain 92, 235-246.

Maximov, A., and Bezprozvanny, I. (2002). Synaptic targeting of N-type calcium channels in hippocampal neurons. J Neurosci 22, 6939-6952.

Maximov, A., Sudhof, T. C., and Bezprozvanny, I. (1999). Association of neuronal calcium channels with modular adaptor proteins. J Biol Chem 274, 24453-24456.

McCleskey, E. W., and Gold, M. S. (1999). Ion channels of nociception. Annu Rev Physiol 61, 835-856.

Miljanich, G. P., and Ramachandran, J. (1995). Antagonists of neuronal calcium channels: structure, function, and therapeutic implications. Annu Rev Pharmacol Toxicol 35, 707-734.

Navaratnam, D. S., Bell, T. J., Tu, T. D., Cohen, E. L., and Oberholtzer, J. C. (1997). Differential distribution of Ca2+-activated K+ channel splice variants among hair cells along the tonotopic axis of the chick cochlea. Neuron 19, 1077-1085.

Pan, J. Q., and Lipscombe, D. (2000). Alternative splicing in the cytoplasmic II-III loop of the N-type Ca channel alpha 1B subunit: functional differences are beta subunit-specific. J Neurosci 20, 4769-4775.

Petruska, J. C., Napaporn, J., Johnson, R. D., Gu, J. G., and Cooper, B. Y. (2000). Subclassified acutely dissociated cells of rat DRG: histochemistry and patterns of capsaicin-, proton-, and ATP-activated currents. J Neurophysiol 84, 2365-2379.

Regan, L. J., Sah, D. W., and Bean, B. P. (1991). Ca2+ channels in rat central and peripheral neurons: high-threshold current resistant to dihydropyridine blockers and omega-conotoxin. Neuron 6, 269-280.

Renganathan, M., Cummins, T. R., and Waxman, S. G. (2001). Contribution of Na(v)1.8 sodium channels to action potential electrogenesis in DRG neurons. J Neurophysiol 86, 629-640.

Ritter, A. M., and Mendell, L. M. (1992). Somal membrane properties of physiologically identified sensory neurons in the rat: effects of nerve growth factor. J Neurophysiol 68, 2033-2041.

Rosenblatt, K. P., Sun, Z. P., Heller, S., and Hudspeth, A. J. (1997). Distribution of Ca2+-activated K+ channel isoforms along the tonotopic gradient of the chicken's cochlea. Neuron 19, 1061-1075.

Saegusa, H., Kurihara, T., Zong, S., Kazuno, A., Matsuda, Y., Nonaka, T., Han, W., Toriyama, H., and Tanabe, T. (2001). Suppression of inflammatory and neuropathic pain symptoms in mice lacking the N-type Ca2+ channel. Embo J 20, 2349-2356.

Saegusa, H., Matsuda, Y., and Tanabe, T. (2002). Effects of ablation of N- and R-type Ca(2+) channels on pain transmission. Neurosci Res 43, 1-7.

Schroeder, J. E., Fischbach, P. S., and McCleskey, E. W. (1990). T-type calcium channels: heterogeneous expression in rat sensory neurons and selective modulation by phorbol esters. J Neurosci 10, 947-951.

Scroggs, R. S., and Fox, A. P. (1992a). Calcium current variation between acutely isolated adult rat dorsal root ganglion neurons of different size. J Physiol 445, 639-658.

Scroggs, R. S., and Fox, A. P. (1992b). Multiple Ca2+ currents elicited by action potential waveforms in acutely isolated adult rat dorsal root ganglion neurons. J Neurosci 12, 1789-1801.

Shin, J. B., Martinez-Salgado, C., Heppenstall, P. A., and Lewin, G. R. (2003). A T-type calcium channel required for normal function of a mammalian mechanoreceptor. Nat Neurosci 6, 724-730.

Simen, A. A., Lee, C. C., Simen, B. B., Bindokas, V. P., and Miller, R. J. (2001). The C terminus of the Ca channel alphalB subunit mediates selective inhibition by G-proteincoupled receptors. J Neurosci 21, 7587-7597.

Soong, T. W., DeMaria, C. D., Alvania, R. S., Zweifel, L. S., Liang, M. C., Mittman, S., Agnew, W. S., and Yue, D. T. (2002). Systematic identification of splice variants in human P/Q-type channel alpha1(2.1) subunits: implications for current density and Ca2+-dependent inactivation. J Neurosci 22, 10142-10152.

Tominaga, M., Caterina, M. J., Malmberg, A. B., Rosen, T. A., Gilbert, H., Skinner, K., Raumann, B. E., Basbaum, A. I., and Julius, D. (1998). The cloned capsaicin receptor integrates multiple pain-producing stimuli. Neuron 21, 531-543.

Vanegas, H., and Schaible, H. (2000). Effects of antagonists to high-threshold calcium channels upon spinal mechanisms of pain, hyperalgesia and allodynia. Pain 85, 9-18.

Williams, M. E., Brust, P. F., Feldman, D. H., Patthi, S., Simerson, S., Maroufi, A., McCue, A. F., Velicelebi, G., Ellis, S. B., and Harpold, M. M. (1992a). Structure and functional expression of an omega-conotoxin-sensitive human N-type calcium channel. Science 257, 389-395.

Williams, M. E., Feldman, D. H., McCue, A. F., Brenner, R., Velicelebi, G., Ellis, S. B., and Harpold, M. M. (1992b). Structure and functional expression of alpha 1, alpha 2, and beta subunits of a novel human neuronal calcium channel subtype. Neuron 8, 71-84.

Witcher, D. R., De Waard, M., Sakamoto, J., Franzini-Armstrong, C., Pragnell, M., Kahl, S. D., and Campbell, K. P. (1993). Subunit identification and reconstitution of the N-type Ca2+ channel complex purified from brain. Science 261, 486-489.

Xie, J., and Black, D. L. (2001). A CaMK IV responsive RNA element mediates depolarization-induced alternative splicing of ion channels. Nature 410, 936-939.

Xie, J., and McCobb, D. P. (1998). Control of alternative splicing of potassium channels by stress hormones. Science 280, 443-446.

Xu, W., and Lipscombe, D. (2001). Neuronal Ca(V)1.3alpha(1) L-type channels activate at relatively hyperpolarized membrane potentials and are incompletely inhibited by dihydropyridines. J Neurosci 21, 5944-5951.

Each of the foregoing patents, patent applications and references is hereby incorporated by reference.

While the invention has been described with respect to certain embodiments, it should be appreciated that many modifications and changes may be made by those of ordinary skill in the art without departing from the spirit of the invention. It is intended that such modification, changes and equivalents fall within the scope of the following claims.

What is claimed is:

1. An isolated cell that recombinantly expresses an N-type calcium channel comprising a Ca_{V}2.2 subunit that comprises exon e37a (Ca_{V}2.2e[37a]).

2. The isolated cell of claim 1, wherein the Ca_{V}2.2e[37a] subunit has a human sequence.

3. The isolated cell of claim 1, wherein the Ca_{V}2.2e[37a] subunit has a mouse sequence.

4. The isolated cell of claim 1, wherein the Ca_{V}2.2e[37a] subunit has a rat sequence.

5-6. (Canceled)

7. An isolated neuron that expresses an N-type calcium channel comprising a Ca_{V}2.2 subunit that comprises exon e37a (Ca_{V}2.2e[37a]).

8. The isolated neuron of claim 7, wherein the neuron further expresses a marker of nociceptive neurons.

9. The isolated neuron of claim 8, wherein the marker of nociceptive neurons is Na_{V}1.8.

10. The isolated neuron of claim 8, wherein the marker of nociceptive neurons is vanilloid receptor VR1.

11. The isolated neuron of claim 8, wherein the neuron expresses both Na_{V}1.8 and vanilloid receptor VR1.

12. A method for identifying lead compounds for a pharmacological agent useful in the treatment of disease associated with increased or decreased voltage regulated calcium influx mediated by a N-type calcium channel containing a Ca_{V}2.2e[37a] subunit comprising
providing a cell or other membrane-encapsulated space comprising a Ca_{V}2.2e[37a] polypeptide;
contacting the cell or other membrane-encapsulated space with a candidate pharmacological agent under conditions which, in the absence of the candidate pharmacological agent, cause a first amount of voltage regulated calcium influx into the cell or other membrane-encapsulated space; and
determining a test amount of voltage regulated calcium influx as a measure of the effect of the lead compounds for a pharmacological agent on the voltage regulated calcium influx mediated by a N-type calcium channel containing a Ca_{V}2.2e[37a] subunit,
wherein a test amount of voltage regulated calcium influx which is less than the first amount indicates that the candidate pharmacological agent is a lead compound for a pharmacological agent which reduces voltage regulated calcium influx and wherein a test amount of voltage regulated calcium influx which is greater than the first amount indicates that the candidate pharmacological agent is a lead compound for a pharmacological agent which increases voltage regulated calcium influx.

13. The method of claim 12, further comprising the step of loading the cell or other membrane-encapsulated space with a calcium-sensitive compound which is detectable in the presence of calcium, wherein the calcium-sensitive compound is detected as a measure of the voltage regulated calcium influx.

14. The method of claim 12, wherein the pharmacological agent that specifically reduces voltage regulated calcium influx mediated by a N-type calcium channel containing a Ca_{V}2.2e[37a] subunit is an agent that reduces N-type calcium channel current densities in nociceptive neurons.

15. The method of claim 14, wherein the pharmacological agent that specifically reduces voltage regulated calcium influx mediated by a N-type calcium channel containing a Ca_{V}2.2e[37a] subunit is useful as an analgesic agent.

16. A method for identifying compounds which selectively or preferentially bind a N-type calcium channel containing a Ca_{V}2.2e[37a] subunit comprising,
providing a first cell or membrane encapsulated space which expresses a N-type calcium channel that contains a Ca_{V}2.2e[37a] subunit,
providing a second cell or membrane encapsulated space which expresses a N-type calcium channel that does not contain a Ca_{V}2.2e[37a] subunit, wherein the second cell or membrane encapsulated space is identical to the first cell except for the N-type calcium channel expressed,
contacting the first cell or membrane encapsulated space and the second cell or membrane encapsulated space with a compound, and
determining the binding of the compound to the first cell or membrane encapsulated space and the second cell or membrane encapsulated space,
wherein a compound which binds the first cell or membrane encapsulated space but does not bind the second cell or membrane encapsulated space is a compound which selectively binds the N-type calcium channel that contains a Ca_{V}2.2e[37a] subunit, and wherein a compound which binds the first cell or membrane encapsulated space in an amount greater than the compound binds the second cell or membrane encapsulated space is a compound which preferentially binds the N-type calcium channel that contains a Ca_{V}2.2e[37a] subunit.

17. The method of claim 16, wherein the N-type calcium channel that does not contain a Ca_{V}2.2e[37a] subunit is a N-type calcium channel that contains a Ca_{V}2.2e[37b] subunit.

18. A method for identifying compounds which selectively or preferentially bind to a Ca_{V}2.2e[37a] isoform comprising,
providing a Ca_{V}2.2e[37a] isoform polypeptide or nucleic acid,
providing a Ca_{V}2.2e[37b] isoform polypeptide or nucleic acid,
contacting the Ca_{V}2.2e[37a] isoform polypeptide or nucleic acid and the Ca_{V}2.2e[37b] subunit isoform polypeptide or nucleic acid with a compound, and
determining the binding of the compound to the Ca_{V}2.2e[37a] isoform polypeptide or nucleic acid and the Ca_{V}2.2e[37b] isoform polypeptide or nucleic acid,
wherein a compound which binds the Ca_{V}2.2e[37a] isoform polypeptide or nucleic acid but does not bind the human N-type calcium channel Ca_{V}2.2e[37b] isoform polypeptide or nucleic acid is a compound which selectively binds the Ca_{V}2.2e[37a] isoform, and wherein a compound which binds the Ca_{V}2.2e[37a] isoform polypeptide or nucleic acid in an amount greater than the compound binds the Ca_{V}2.2e[37b] isoform polypeptide or nucleic acid is a compound which preferentially binds the Ca_{V}2.2e[37a] isoform.

19-23. (Canceled)

24. A method for preparing an analgesic agent, comprising
identifying an agent that selectively or preferentially reduces calcium channel current densities in nociceptive neurons mediated by N-type calcium channels containing a Ca_{V}2.2e[37a] subunit, and
formulating the agent for administration to a subject in need of such treatment.

25. (Canceled)

26. A double stranded RNA molecule specific for Ca_{V}2.2e[37a] RNA.

27. The double stranded RNA molecule of claim 26, wherein the molecule is 21-23 nucleotides in length.

28. The double stranded RNA molecule of claim 26, wherein the molecule has a 3′ overhang.

29. The double stranded RNA molecule of claim 28, wherein the 3′ overhang is 2 nucleotides in length.

30. The double stranded RNA molecule of claim 26, wherein the molecule is a single molecule that comprises a hairpin structure.

31. A method for inhibiting calcium influx in a neuronal cell mediated by a N-type calcium channel containing a Ca_{V}2.2e[37a] subunit comprising
contacting the neuronal cell with an amount of a Ca_{V}2.2e[37a] inhibitor effective to inhibit calcium influx in the mammalian cell.

32. The method of claim 31, wherein the inhibitor is selected from the group consisting of an antibody which selectively binds the Ca_{V}2.2e[37a] polypeptide, an antisense nucleic acid that reduces expression of a Ca_{V}2.2e[37a] polypeptide, a siRNA that reduces expression of a Ca_{V}2.2e[37a] polypeptide.

33. A method for treating a subject afflicted by pain mediated by a N-type calcium channel containing a Ca_{V}2.2e[37a] subunit comprising
administering to a subject in need of such treatment an inhibitor of the Ca_{V}2.2e[37a] polypeptide in an amount effective to inhibit voltage regulated calcium influx and thereby to reduce the pain.

34. The method of claim 33, wherein the inhibitor is selected from the group consisting of an antibody which selectively binds the Ca_{V}2.2e[37a] polypeptide, an antisense nucleic acid that reduces expression of a Ca_{V}2.2e[37a] polypeptide, a siRNA that reduces expression of a Ca_{V}2.2e[37a] polypeptide.

35. The method of claim 33, wherein the inhibitor is administered prophylactically to a subject at risk of being afflicted with pain.

36. The method of claim 33, wherein the pain is neuropathic pain.


SEQUENCE LISTING (47 sequences)

SEQ ID NO: 1 - DNA, 24 bp, Artificial
  PCR primer
gctgcgtgttgccggattcattat

SEQ ID NO: 2 - DNA, 24 bp, Artificial
  PCR primer
ttcattcgaaccaggcgcttgtag

SEQ ID NO: 3 - DNA, 24 bp, Artificial
  PCR primer
ctgaatacgacccagctgcgtgtg

SEQ ID NO: 4 - DNA, 24 bp, Artificial
  PCR primer
ccaggcgcttgtatgcaactcgag

SEQ ID NO: 5 - DNA, 22 bp, Artificial
  PCR primer
cgcaggttctggagccttagct

SEQ ID NO: 6 - DNA, 22 bp, Artificial
  PCR primer
ggccattgctgtggacaacctt

SEQ ID NO: 7 - DNA, 28 bp, Artificial
  PCR primer
cattctggacttcattgttgtcagtgga

SEQ ID NO: 8 - DNA, 28 bp, Artificial
  PCR primer
tcgcaggactctcagagacttgatggta

SEQ ID NO: 9 - DNA, 25 bp, Artificial
  PCR primer
cagagatgcctggaacgtctttgac

SEQ ID NO: 10 - DNA, 25 bp, Artificial
  PCR primer
ataacaagatgcggatggtgtagcc

SEQ ID NO: 11 - DNA, 30 bp, Artificial
  PCR primer
agctgcgtgttgccggattcattataagga

SEQ ID NO: 12 - DNA, 30 bp, Artificial
  PCR primer
gcgcttgtaggccaacctacgagggcagtt

SEQ ID NO: 13 - DNA, 30 bp, Artificial
  PCR primer
agctgcgtgtgggcgcatcagttacaatga

SEQ ID NO: 14 - DNA, 30 bp, Artificial
  PCR primer
gcgcttgtatgcaactcgagccgggcattt

SEQ ID NO: 15 - DNA, 26 bp, Artificial
  PCR primer
tcaattcccacacacctcccagttcc

SEQ ID NO: 16 - DNA, 26 bp, Artificial
  PCR primer
tttgggtggtctgcttagggaaccag

SEQ ID NO: 17 - DNA, 24 bp, Artificial
  PCR primer
gcgctggttgtaagggtcagattg

SEQ ID NO: 18 - DNA, 24 bp, Artificial
  PCR primer
agccaggcaacaattgcagaaatc

SEQ ID NO: 19 - DNA, 22 bp, Artificial
  PCR primer
cagcaccagcatcaccccattt

SEQ ID NO: 20 - DNA, 24 bp, Artificial
  PCR primer
caagatggtgaaggtcggtgtgaa

SEQ ID NO: 21 - DNA, 135 bp, Artificial
  Competitive template for exon 37a
agctgcgtgttgccggattcattataaggataagcgtgctggactctctcgacgcaaatcgaggcccaattggcaaatacagttgcagaacaggaggagccacacaactgccctcgtaggttggcctacaagcgc

SEQ ID NO: 22 - DNA, 135 bp, Artificial
  Competitive template for exon 37b
agctgcgtgtgggcgcatcagttgcaatgataagcgtgctggactctctcgacgcaaatcgaggcccaattggcaaatacagttgcagaacaggaggagccacacaaaagcccggctcgagttgcatacaagcgc

SEQ ID NO: 23 - DNA, 9562 bp, Rattus norvegicus
  exon (1)..(128): Exon 36, continues 5′ to nt 1
atgctgaacctctttgttgctgtgatcatggacaatttcgaatacctcacgcgggattcttccatcctagggccgcaccacctcgatgaattcattcgcgtctgggctgaatacgacccagctgcgtggtgagtgagctccagtgctctctgctcctcagggttggctgccattgcctgtggttctccagacagggccagggtgatcttagacagttccatgtgcatgtccctaggtgttgggctgaggtctagtgacttacccagttctgtgtgtggtagcactggtccccttgcagtagcatgtttcttttgatcccgtgccttggtgcagctgtggggcagagacactaagtatggtcctcaaagagagtgcctatgcctgggggtcaaggaacatttccagggctctttgcacagaagtgtcctgccttctcccaaggcttgtcttgtgatggaacatggaggcccagtggcggagggtagagagagagtgtagggacacatctgtgtaatactcagagggtgttgagagcaaatgatgtgtgtgtatgtgtttgggaggggtgtggttgctgctgtggctgttgttgtttaaagaattggggactgaaccctgggacatgtaagtcaaaatactttagcaactgagctgtatcccccacttgccataatgtgagtcttaaattttttcttgatggatttagtttttcttctgccatttcttttggggccaggagccagtgtgtatgtctgttgccagccttgttttatgtggtgggcactgggtggaactttattcaaaccaggggaattggggaaatagaaaactgttaccgggctgcataagtgaagggtgatttgtttctagatagcaaggaccttctggattgctttgggtgaaagtgaaagttgtttgaagccatgttccagagagagcattgcacagtgatgtcatggtccatagagaacactgcacagtgatgtcatggtccaaagggagcactacacagtgatgtcacaatccggagagagcactgcacagtgatgtcacagtnnnnnnnnnnacacagtgatatcacagtccatagagagcactgcacagtgatgtcacagtccatagagagcactgcacagtgatgtcacggtccaaagggagcactacacagtgatgtcacagtccatagagagcactgcacagtgatgtcatggtccaaagggagcactacacagtgatgtcacggtccatagagagcactgcacagtgatgtcacggtccatagggagcactgcacagtgatgtcacggtccatagggagcactgcacagtgatgtcacggtccatagggagcattacacagtgatatcacagtccatagagagcactgcacagtgatgtcatggtccatagggagcactgcacagtgatgtcatagtccatagagaacactacacagtgatgtcacggtccatagggagcactgcacagtgatgtcacagtccatagagagcactgcacagtgatgtcacagtcaatcaataatactgcacgatgattgtcacaccaatcccctttgccagagaccacattcaggaaggcaggggagcaggacatcggggctctccatccgtgtgcttgttcgtccctctacccacttggtcaccgaattgatgcactcattgcgtctgctcctacgccccaccatctcctctcacctacctgctcacttgtgtattgtgttagttaccttttgattctgtgattggatctcacaaaagcaacttcaggcaaaggcttgctttggcttatgatttaagaggagatatagtctaacacggaaggaaggcacatggcaggagcaggagaccgtgggtcacaatcatgggagatgagtgctggtgtccatctcagtttactttttattcagcccgggaccctgacctgggatgatgccaagtacatttattgcgtggctcttcccatctcaatccaatgtagccgttccctcacagagagggcgctcagaggcctgccttctgggtgcctcttgaccccgttctgctgacacccacacatgcgctcacccaggtattttccatttaatggctcattcgcccattcatttgttcagccgctcaagtggtatttgcagacggctcttccttgtaagggggcgtgcaggaggggcagaggctcagagcactgactgctgtggaagatacaagcctctcctctcacctgccacagctgtgaatgggcagttgtccttgaaggagtgagtggtggcgtcctgtgccttagggatggacaagaaactgcagctgtgtgcagttcctcaggcccccttgtctgcctctcctaggttgtggtcagcgttttgctgtgggatagttgggagaatgaagtcaggctcctgtggttagactgggcatgctaagctttctggtagaccctgggggctgaagtcaggacaggcatgcttaggtctctgctggaggactagaggatgtgaccaggctgatggctagagtcctctgtctcaagctcctaagcttctgcgtccttgacctgatgcccagcacaagatgctcagtaaactccgggaaggggtagtcaggctttcttctgctgggttagatccccattgcttctgtccttcagcctctcctctgcgcctagagacagatagatagactgcaggtgtcttcagttcctcagctgcccttgtctgcccctcacagtgtgttctgaggttgtgcctggagttgtagccagggaagtttcacagtattgcatcagggtgctgggacagaggtcttttccgttggggctgtttggactgacgtcctctctgagtctatttcccattgctgtgagaaacctcccatttttacatagttcataagcagcacttattgttgttgtgcattacgcttgactactttgactctgtagacgattctctgtctatattcatatgttttccccaccatgcccaagactcttactgagctgctcaggctcaaggatattgcactagctctgtggttaggtatgtcaccgaggtagggtgcagtggggagactggactttgtcatataaagggcaggagagactccagcggcaaaatgactcatttgatcttcaagattaccctggctatgacctcaacagccattgctatttctggaatattttcccaaggtgcaaagaacagatggtctgtgggactcactcacactgaaggccagctgtgaagggcagcatggtgcttgccctcatgaactgcatacccagttcctgccatcgactgtaacgaagaaggggtaacgggcacagtggtgcttagcctgaggacagacttgggagcgtgcacagtgagagtgtagtctgtcttctaacttgagagctagagagctaggggccctggatgtcaccagcaagttgaagagatggccttgtggtaggggtgacaccatggccaaacggcatccagatcaagccgcttaaagatcaggtgaacttcaaagtgctttgagactggtgcttgttccctgcagacccctttaggaggtggatggcttgacagagcactcaaacaggtcaaagctggcagagcaccatgctaaagctgggtcctgcatttgaacaggcttacggtctgtcttaatttcctatgtgaccatgtgattttctcatcatttgggtaaaactttgaaacgacaaactgtcagactttctaacatttgtgtatgtgtctgcacaagcacagatgtgtgtgagcatatgcatgtgtgtatatgtggatgagcctgtgtgtcctctgcatgtgcacacaaaggcacatgcatatgtttgtgtatccatgttagtttgtgtgtgtgtgtgtgtgtgtgatacacatctgtgtatggtaagtatacatgtgtgttgtgtgcatgtgtgagtcattccatgtatttgtgcattcctgttaggtgtttatgtaccacctgtacgttgtgtgaacgtgtacatgctgtgggatactagaaaggcatggagcttctgggagctggagctgctctttccccggcctcccttttgttgcttcaaaacactgtgttgaagcatggggaccgaaagtgaaagccatgcttaccggtcgtccatctgctgaggaaacacttcctgagcatctgctctgtcctgggcctggggccccaaaggtgaaagtgtaaatagggctgtgagataggacagttagatgttcacagcttggggccagggcacctttgaggagggaaggatcacgtgcacctaagctgtgtgaaaggaggcaagatgaacggagtgtcaggcaactctgaggcaggtacagagagagggagaactgggaagatcgttgaggcttgagtgaaaatgttaaagctgggagagattgagagaccttgaaactgaagttaaacaggtaattttgcttttgttttacttgggaaaatattcccaaacagggttaacgtgtatcatgtccacgcatttccactagtgttaaagaatggacttgatgagtagttggccccttggaattgtccctaggatccaacaagattagcaagtatgtgaggcccaagtagaatatcctcttttctggaagcaaactgagcacgacttaaagcttgatgagcagcataggagaaagaagggtctgctgtgccaaagggtatcatccccgtgaggagggcttttctagttaggccagcaaactcccagggaacaccagtgtgttcccagggagatgggaacaatcgagaggcatctaggtgtgtattcctcagtagggtttctgtgtctgagaaatggaggggacacatcacaggagtaagggccagggtgagagggagacaaagagaaaggctctggcattcaggaactgagggcaggacagacagcaagtccagttgggactggtcagtggtggggctcatggtgggcgtggggtgtttgttcttctgcattattctgagtagttcctgcttagcctcacatccaggcattatcttggggctgacactttctggggagcactggacttcaggtcccactagggagtagcctgtgtagctgtacccctcacctccacctcacagctcatcttccattgcagcctgggtgggtgggggacgggttccatcctgcctgttgccgtaacctgtgacatttcctttccagttgccggattcattataaggatatgtacagtttgttgcgttgtattgcgccacccgttggcttagggaagaactgccctcgtaggttggcctacaaggtttgtaacttcagagcctccatctgacataccccccgcccgaatcgcacacactgcacacactccctgcctgacttgggaatgacaacacctcaccttctctttcctctgtcctgccctgccctgcgttccagcatcctggatgctttggttccctgtcttatgtcccctagctggaggcaggcagccttcatgacagtgtctgagatggtttctggcctggctcggcggagtctgccaggaccaattggctgggcgtgggggttagggagggggttggcatgtggttgagacaggtcccacctttaccaagcaaacagccatcacacccagcaatcctgcaggctcttatgattgggctgatggtccgacagccagaggctgagagccgagaggagaatttgtacatgggtgcaaataaacaaggagcagttgtccctgtgggtcaggagaggctctcaggtccctgggggaggggagagtgggaacaagaaaggggagcccccctgcgtcgcttccggattgatgtgctcagctcagggagaaaacccctggcctggaactgtgagtgaagggcagggtctgggcagatctgccctctggagaaaggctggactctgctgtgtctctgagaccccaagcccacaggcaggtgctctggggtagagggaagccccagcctcttgacttgggaagatgctacaccagccccaagatgctgccctgagtgccccagcccacaggggcgcatccaacctgttggtcccaacctgtgctgtcagaggcactagtggcaccaggccacagcttactcagctcctcccttcccgcagtctccttcccatccagccctgacttcagcagcagccctgccctgggctggcctcgggcgtctctgggcttggagctgacttgccctgtcattcagttcagtcactaagaactttgggactccctttctagctttctaatctgttgtagacttttctccacattagcctccggccttatgtggaagcaaatccaatctgcccattgaggtgggcgacactgctgctgtggttctcagcggtagtctcctctccacagtctggccagtgcttcccgtgccatgaaccagctctttctgatttggccttgtctgcagttctaaaacagtaaagtcagaaatttataacccaaagccgccccgatgcagcccttcctcacccctccatgttcattagctcagacatcacccctgcccgctgccctgcattgggctgcctatgaggaggtgaggctgggggtgggggcaagcaagggtcaagtcaagcagagataccttcattttacttgtttttgatttggggcatttacccgaactttggttaactgaatctgttttcttgtctgcttcctcccctgcccgctccctgctctgcccctctgccgtgcgcctctcctgtggaccaccccactatgcctggcctctggaacgggtttccagtgggcgcatcagttacaatgacatgtttgagatgctgaaacacatgtccccacctctgggtttggggaagaaatgcccggctcgagttgcatacaaggtagaccttgaccctgcactgatcctgcatccaacccaggagctgtctgtgcttggctgggcccacctggccctcattcagcccacagagcactcatgggctctcacctgcttccttaggatttgtctggctgtctggctgtacctggctgccttactgctgctccctcaccaccccgaaatcctttgctcccatcccagacagaggcatacgtctctgcactaatccactaccctgtctgacttccccaccactgacatccttgaggagcttgggacaatggagccagagataccccatccagggcccttttcccttctttgcctgtgctttcccaagaatgagaagaccaacaaaacaatgcttttggaccgagggtgctttcctcctagatgccctgcccctgtgctcctgtcatgggtggtcatcaagtctgcaaggtctgcctccagggacagatggggcctgcgggcctctgctgctcccctgtgccgtgcccttctaccccagctaagctcagccttgtagggtttgtgcccagctagtcttcaagggcttcaaaacttcccagtgggaaggaggtggggccttgacgttggaagggaaaggctgcttggatgccttggttaaggctccagcttcgcattcaggagtcccagcacacgggagtcgtgaggaaggaagtgaacatggacacctccaggcctccttcctggcccagtcatagctcgctgcacctgcagtgccctcagcactcatagcccctactgctccccactaaacagtgccaactgtgttagcatctgtctgagagctccctgctggctcctccctcttcagcccgctgtactctgtccatcttctgtctccgtaccccattctaggtttttttgtctgccacttcattcagtgtagtctgtgggaaccaaacagctctcccaccactcacagatgcctctgggttcttcctgcctttatctgcccccaactagcatgtgtgttgaaactgcggaggccttctgcatgcatgcgtgcatgtctctacccacaggagggagagctctgttcctatgacacccagtcagacagaaaggcaggcaggaagggttgcactgccctttccagccaggcctgaggatcctgaggttcagggtgtgaccagaccaagagaggcttcggggtaggttgcagatggcttggggcaaaatgggggagggcccaagagatggttccagtaccatttctggaacttatttgccatggtcttgccactctgcttctctgaggttctatctttctctgtcctccatgactgggggcaaacctgtgggagcccctgtgagggtgacctttggtgtcctggacagatctccccctcttggaaacatctgcttgctcttctctcattcccatcctgacccctccctggaagaggaatttcttggcctctcacactgccatcggtgctggtctgcagggacgccatgctccgggaagcaggcttttcctggtgtgtaacacatgtgacacgtgaggtcctggtcctgtgattggacgtgcccagtggggctttgccagcattcagaggacaagagcctacctcctgtgttctgcaccccatgcccctctatcacaatgaagatacagaacgtgggggcaagatcctagggaccgggctgtgtggcccttccctgaagaagggtgcttgccttagggaggaagtcctatctctagttttcttcctatacagaggacattagtattcctggggtgcttcatggccgactgagtggtntcagatgcacctgagacctccctcagatgttntgagagccggacttccttgcagctgctgagtctacccagaggaatgactcagacacatccctgtttcaagatgtgttgtctctgaaaaacaagaaaagaaagggagctggccactcagagatataggagcagcttgctctgctctggcccttagaaaggtctcttaagactcttcttcattcatttacctatgatggtgtctgtcttcaagtgtgtctccccggccccctgtttgtggttaagacctgagttaagcaagagcatctctcttgatgagtgcctcgttgggactggacctcagagaggcctcttggcttccaccccttacttacccaatgatccagttgcagatagagggcaggtaagcaaaaaccacaggacagggcctccaactgtattctaagctgtcccactctgccttcctcagccaggaccatgcccccttgtgtcactcatagccagtggtcaggactcaggctcctgggcctggcttctgctacagcttctacttggacttctgttcttctctagtcaggagaatctcagcttagatgagagtaaggtccaggaggagagaatgctggcaaaacttccaccgcccagccttctggaccaggcacctgccaggaccactctcgggacaccaagccaagctccagagcccagacactaactctagggtgggagtgaccgtaatatgcttggccctgatctgcccctcctcatccccagcgcctggttcgaatgaacatgcccatatccaatgaggacatgacggtacactttacatccacactgatggccctcatccggacggcactggagatcaagcttgccccag

SEQ ID NO: 24 - DNA, 334 bp, Rattus norvegicus
atgctgaacctctttgttgctgtgatcatggacaatttcgaatacctcacgcgggattcttccatcctagggccgcaccacctcgatgaattcattcgcgtctgggctgaatacgacccagctgcgtgttgccggattcattataaggatatgtacagtttgttgcgttgtattgcgccacccgttggcttagggaagaactgccctcgtaggttggcctacaagcgcctggttcgaatgaacatgcccatatccaatgaggacatgacggtacactttacatccacactgatggccctcatccggacggcactggagatcaagcttgccccag

SEQ ID NO: 25 - protein, 111 aa, Rattus norvegicus
MLNLFVAVIMDNFEYLTRDSSILGPHHLDEFIRVWAEYDPAACCRIHYKDMYSLLRCIAPPVGLGKNCPRRLAYKRLVRMNMPISNEDMTVHFTSTLMALIRTALEIKLAP

SEQ ID NO: 26 - protein, 111 aa, Rattus norvegicus
MLNLFVAVIMDNFEYLTRDSSILGPHHLDEFIRVWAEYDPAACGRISYNDMFEMLKHMSPPLGLGKKCPARVAYKRLVRMNMPISNEDMTVHFTSTLMALIRTALEIKLAP

SEQ ID NO: 27 - DNA, 9695 bp, Rattus norvegicus
  CDS (92)..(7093)
cggcacgagcggctaggttaggagcccctggcgcgccgcgccctcggtgccgggccgcggagccggggatgcgcgcggcgccccgggagtcatggtccgcttcggggacgagctaggcggccgctatgggggcaccggcggcggggagcgggctcggggcggcggggccggcggggccggtggcccgggccaggggggtctgccgccgggccagcgggtcctgtacaagcagtccattgcgcaacgcgcacggaccatggccctgtacaaccccatcccagtcaagcagaactgcttcaccgtcaaccgctcgctcttcgtcttcagcgaggacaacgtcgtccgcaaatatgctaagcgcatcaccgaatggccgcccttcgaatatatgatcctggccaccatcatcgccaactgtattgtcctggccctggagcagcacctccctgatggggacaagactcccatgtctgaacgactggatgacacggaaccttacttcatcggcatcttttgcttcgaggcgggcatcaagatcatagctctgggcttcgtgttccacaaaggctcctacctccggaatggctggaacgtcatggacttcgtggtggtcctcacagggattcttgccacagctggaactgactttgatctgcgcaccctgagggctgtgcgtgtgcttaggcccctgaagttggtgtctggaattccaagcttgcaggtggtgctcaagtccatcatgaaggccatggtcccgctgctgcagatcgggctgctgctcttcttcgccatcctcatgttcgctatcatcggcctcgagttctatatgggcaaattccataaggcctgcttccccaacagcacagatgcagagcctgtgggtgactttccttgtggcaaggaggcccctgctcgtctgtgtgacagtgacaccgaatgccgggagtactggccaggacccaactttggcatcaccaattttgacaacatcctgtttgccatcttgaccgtgttccagtgtatcaccatggagggctggactgacatcctctacaatacaaatgatgcggccggcaacacgtggaactggttgtacttcatccccctcatcatcattggctccttcttcatgctcaacctggtgctcggtgtgctttcaggagagtttgccaaagagcgggagcgagtcgagaaccgccgtgccttcctgaagctccgcaggcagcagcagattgagcgagaactgaatgggtacttggagtggatcttcaaggcggaggaagtcatgttggcagaggaggacaagaacgcagaagagaagtcccctttggatgtgttgaagagagctgctaccaagaagagccgaaatgacctcatccatgcagaagagggggaggaccggtttgtagacctctgtgctgctgggtctccctttgctcgtgccagcctcaagagtgggaagacagagagctcatcgtacttccggaggaaggagaagatgttccggttccttatccgtcgtatggtgaaagcacagagcttctactgggtggtactgtgcgtggtggccctgaacacgttgtgtgtggccatggtacactataatcagcctcagcggcttaccactgcactgtactttgcagagtttgttttcctgggtctcttcctcacagagatgtccctgaagatgtacggtctagggcccagaagctacttccggtcttccttcaactgctttgactttggggtgattgtggggagtatctttgaagtagtctgggctgccatcaagccaggaacctccttcggaatcagtgtgctgcgggctctccgactgctgaggattttcaaagtcaccaagtattggaactccctgaggaacctggttgtttccctcctcaactccatgaagtccatcatcagccttctcttcctgcttttccttttcattgtggtcttcgctctgttggggatgcagctgtttgggggacagttcaactttcaagatgagactccaaccaccaattttgataccttcccagctgccatcctcactgtgtttcagattctgacaggagaggactggaatgcagtcatgtatcatgggattgagtcacaaggaggagtcagcaaaggcatgttttcatccttttacttcatcgtcctgacactgtttggaaactacaccctgttgaacgttttcttggccattgctgtggacaaccttgccaatgcccaggagttgaccaaggatgaagaggagatggaagaggcagccaatcagaagcttgctcttcagaaggccaaagaagtagctgaagtcagccccatgtctgctgccaacatctccattgctgccaggcagcagaactcggccaaggcgcgctcagtatgggagcagcgggccagtcagctaaggctccagaacctgcgtgccagctgtgaggcactgtacagtgagatggacccggaggagcgcctgcgttatgccagcacgcgccacgtgaggccagacatgaagacacacatggaccgacccctagtggtggaacctggtcgggatggcctgcggggacccgccgggaacaagtcaaagcctgagggcacggaggccaccgaaggtgcggatccaccacgccgacaccaccggcatcgtgatagggacaagacctcagcctcaacccctgctggaggcgaacaggacaggacagactgcccaaaggccgaaagcaccgagaccggggcccgggaggaacgtgcgcgccctcgtcgaagtcacagcaaggaggctccaggggctgacacacaagtgcgttgtgagcgcagtagacgtcaccaccggcgcggatccccggaggaggccactgaacgggaacctcggcgccaccgtgcccaccggcacgcacaggactcaagcaaggaaggcaaggagggcactgcaccggtgcttgtacccaagggcgagcgtcgcgcaagacatcgaggcccgcgtacgggcccccgtgagacagagaacagtgaggagcccacacgcaggcaccgtgcaaagcataaggtgccaccaacacttgagcccccagagagggaggttgcagagaaggagagcaacgtggtggaaggggataaggaaactcgaaatcaccagcccaaggaacctcgctgtgacctggaggccattgcggttacaggcgtgggctctctgcacatgctgcccagcacctgtctccagaaagtggacgaacagccagaggatgcagacaaccagcgtaatgtcacccggatgggcagtcagccctcagaccccagcaccactgtgcatgtcccagtgacactgacaggccctcccggggaggccactgtagttcccagtgctaacacggacctggaaggccaagcggagggcaagaaggaggcagaggctgacgatgtgctgagaagaggccccaggcccatcgttccctacagttccatgttctgcctcagccccaccaacctactccgtcgcttctgccattacattgtgaccatgcggtactttgagatggtgattcttgtggtcatcgccttgagcagcattgccctggctgctgaggatcccgtgcggaccgactcattccggaacaatgctctgaagtacatggactacatctttacaggagtcttcacctttgagatggtcataaagatgatagacttgggcctgctgctgcaccctggggcctacttccgggacctgtggaacattctggacttcattgttgtcagtggagccctggtggcatttgcattctcaggatccaaagggaaagacatcaataccatcaagtctctgagagtcctgcgagtcctgcggcccctcaagaccatcaagcggctgcctaaactcaaggctgtgtttgactgtgtggtgaactctctgaagaatgtcttgaacatcctgatcgtctacatgctcttcatgtttatatttgccgtcatcgccgtccaactcttcaaagggaagttcttttactgcactgatgagtccaaggagctggagcgggactgcaggggtcagtatttggattatgagaaggaagaggtagaagcccagccaaggcagtggaagaaatatgacttccactatgacaatgtgctctgggccttgctgactctgtttacggtgtccacaggagaggggtggcccatggtgctgaaacactctgtggacgccacctatgaggagcaggggccaagccccgggtttcggatggagctttccatcttctatgtggtctactttgtggtcttcccttttttctttgtcaacatctttgtggccttgatcatcatcaccttccaggagcagggggacaaggtgatgtctgagtgcagtctggaaaagaatgagagggcttgcattgactttgccatcagcgccaaacccctgacacggtacatgcctcagaacaagcagtcgttccagtataagacatggacatttgtggtctctccaccctttgagtacttcattatggccatgatagccctcaacacagtggtgctgatgatgaagttctacgatgccccttatgagtacgagctgatgctgaagtgcttgaacatcgtcttcacatccatgttctctctggagtgcatcctgaagatcatcgccttcggggtgttgaactacttcagagatgcctggaacgtctttgactttgtcactgttttgggaagtattactgatattttagtaacggagattgcggaaacgaacaacttcatcaacttgagcttccttcgcctcttccgggcagcacggctgatcaagctgcttcgccagggctacaccatccgcatcttgttatggacctttgtccagtcctttaaggcgctgccctacgtgtgcctcctcattgccatgctgttcttcatctacgccatcatcggcatgcaggtttttggaaacattgcccttgatgatggcaccagcatcaaccgacacaacaacttccggacatttctgcaagccttaatgctgttgttcaggagtgccactggggaggcctggcacgaaatcatgctgtcttgcctgggcaaccgggcctgcgacccacatgccaacgccagcgaatgcgggagcgactttgcctatttttattttgtctccttcatcttcctctgttcctttctgatgctgaacctctttgttgctgtgatcatggacaatttcgaatacctcacgcgggattcttccatcctagggccgcaccacctcgatgaattcattcgcgtctgggctgaatacgacccagctgcgtgtgggcgcatcagttacaatgacatgtttgagatgctgaaacacatgtccccacctctgggtttggggaagaaatgcccggctcgagttgcatacaagcgcctggttcgaatgaacatgcccatatccaatgaggacatgacggtacactttacatccacactgatggccctcatccggacggcactggagatcaagcttgccccagcggggacaaaacagcaccaatgtgatgctgagctgaggaaggagatctcttctgtgtgggctaatctgccccagaagactctggacttactggtgccaccccacaaacctgacgagatgacagtggggaaggtctatgcggctctcatgatatttgacttctacaaacagaacaaaaccaccagagatcagactcaccaagctcctggaggcctgtcccagatgggtcctgtttccctgttccatcctctgaaggccaccctggagcagacacagcccgctgtgctccgaggagctcgggttttccttcgacaaaagagtgcaacttccctcagcaatgggggcgccatacaaacccaggaaagtggcatcaaggagtccctgtcctggggcacgcagaggacccaggacgtactttatgaggccagagcacctctagaacgtggccattctgcagagatccctgtggggcagccaggagcactggctgtagatgtccagatgcagaacatgacattgagaggaccggatggggagccccagcctggcctggagagccaaggccgagcggcctctatgccacgcctggcggcagaaacacagccggcccctaatgccagccccatgaagcgctccatctccacactggctccacgcccgcatgggactcagctttgcaacacagtcctggaccggccacctcctagccaggtgtcccatcaccaccaccaccgctgccaccggcgcagggacaagaagcagaggtccctggaaaaggggcccagcctgtctgttgacacagaaggtgcaccaagtactgctgcaggatctggcctgccccatggagaagggtccacaggctgccggcgggagcgtaagcaagagcgaggccggtcccaggagcggaggcagccctcctcctcttcttcagagaagcagcgcttctattcctgtgaccgctttgggagccgggagcccccacaacctaagccctccctcagtagccaccccatatcgccaacagcggcactagagccaggaccccacccgcagggcagtggttccgttaatgggagccccttgatgtcaacatctggtgctagcacgccgggccgaggtgggcggaggcagctcccccagactcccctgaccccacgccccagcatcacctacaagacggccaattcctcgcctgtccactttgctgagggtcagagtggccttccagccttctcccctggccgtctcagccgcggcctttctgaacacaatgccctgctccagaaagagcccctgagccagcctctagcttctggctcccgcattggctctgacccttacctagggcagcgtctggacagtgaggcctctgcccacaacctgcctgaggatacactcacctttgaagaggccgtggccaccaactctggccgctcctccaggacttcctatgtgtcctccctcacttcccaatcccaccctctccgccgtgtacccaatggctaccactgcactttgggactcagcaccggcgtccgggcgcggcacagctaccaccacccagaccaggatcactggtgctagctgcaccacgaccacccatgcaccagctcgtgggtgcgggttccagttgatgagttttatcatccgctctgggttgtgcggtcacagccctgggaggagggtcctcacatcgcggcctctgtggtggaggttcctgcttctctccctccctcccttttacactggacagactaataaagccctttcttagagggatatggtcctctctatcctcctgtgtactgccttcctgggttccatgccagatgttggatcctaagcagaggtagctgagttgagatagacccagcaaatccaaatcctatgtcatggcctccagcttccagggtgggtacttgggactttcttaggaggtctgagcctcatggagattgtggtttgtccaaatgtgtggcatgggggatagggtaccctcaaaggcaaggaaaggagcccaactgtgtggcctggcagcacctgccagcatcactactctcatgtctattgtggtcttggagtcaaacagcacatgtatatagagatatgctcaagggcctgcctttcacctacattgtcaccataatagggaccaaatctagaggatgtccttgctgttgattctggttttcagtcacaacactttcactttttgtcatttctatatagttgatctagaaaaacagaaatcaaaacagggaagaaaatgttcgtgtaacttaaaaaagaaatcaacgtgtaggaaggtctccattttgcattgtttctgtgacttgtatgcaatgttcctgtatgtattctacccttcccgggaagtccccaatgaccctggttcctctgctcaaccaagtgcctgatctctggctctgagcatcgtggctgaggtgcggcctcaggaagcatcggggagctgctcagagcagcactaggacttgtgtcttagggacactgaccgtgtccagcagcatgtcagagaagcagctgtagtgcccatgttcctccctgagtgatgggttctgaagaagccagagcagcacaatgtgtgcttgcgtgaggcactttccgccttttaaaatctgattctcagggatgggatgcctgccaagtagggtgtgatctctgttgtgttttaaaaaacaacaacaacaaacaaacaaaacctagtattcactgaatgctgaagagagcaaaatgcaagcaaagaagggactggggttagagggagaagcccgcactggcagcataataagaaactggcagggaggggatggtcctggaacaggccaggtgcctagagctgagtccagcccctggcccggaactggggacacagcactcaaataaaacctcatggctacttggtgaaaggcaaacccatgctcaggaaggtgttcagtgtgcagagatggctgtgaggccatgagagaaaggtttcacataggcaggcagtccttggtgtgttctctgtgttttgaaacgtctgatgacttcttggtggactgttggtttctaccccatgtttctcacagaagctgtgtatatgtgtgattgcgcgtgtgattgcatgtgtgtggtagtgtgcgtgcgtgagcatgcatgagtcataggaaatgtgtgtgtgtgtgtgtgtgtgtgtgtaggtgtgtgtacgtgtgttcagcaagtggcttttgtcaaccatagggctatgcaacaaaagacacattactagaaacaaaacacaagaccaccactcggtctagggtttcagcatgattgtgaccaaaccttttatagaatttccttatatgaaggcacaataccctgaaactttaaagataacagagtattttattccagtagggtaagattaaacaggaccctggactgcatgtgactgcactcatgtacaacagaggaggatgtgcattttgatactgttctgtctctgtcccagccccagcccttttctcttgagtgttgaatgtatacattctgtgtggaactacagctgctccagacagtcctgggttgggaatcatctttatcccacattaacatagctggcttttcttccaagcactggtacacaggaaaggagacatgatgtcttgcttcctgactttgggtttgtttctgtactgtctcttctcaagatgttgtctgttccccctgaaatttcatagtgagttgccaaatttgaaatgcaacaaccagctgtctgcatctggaacctgtcaagcagtgctgtagtttgaaaaagttatgtgtgcatgtaaaatatacacatatatatatatacattatacaagtatgtgcatgaaatgtatatcttcatactttttgatacaatgtattcatttgttaatttttaattatatttgatataaattgaaggtttgttgcaaaaatttatatttaacagtgttgagagagagagaaagagcgagagagggagagagagagaaagatccaatcatgcaacagaaatgggactactttaaaaatcagtcctttgactagtttgctgccctgaataatatttacaaaccaaactttggattctgctcttgtttctacaatgactttttgtataaagcaaagtccttggattaataaaacaaccaaaaatcaaattaaaccatta

SEQ ID NO: 28 - protein, 2333 aa, Rattus norvegicus
MVRFGDELGGRYGGTGGGERARGGGAGGAGGPGQGGLPPGQRVLYKQSIAQRARTMALYNPIPVKQNCFTVNRSLFVFSEDNVVRKYAKRITEWPPFEYMILATIIANCIVLALEQHLPDGDKTPMSERLDDTEPYFIGIFCFEAGIKIIALGFVFHKGSYLRNGWNVMDFVVVLTGILATAGTDFDLRTLRAVRVLRPLKLVSGIPSLQVVLKSIMKAMVPLLQIGLLLFFAILMFAIIGLEFYMGKFHKACFPNSTDAEPVGDFPCGKEAPARLCDSDTECREYWPGPNFGITNFDNILFAILTVFQCITMEGWTDILYNTNDAAGNTWNWLYFIPLIIIGSFFMLNLVLGVLSGEFAKERERVENRRAFLKLRRQQQIERELNGYLEWIFKAEEVMLAEEDKNAEEKSPLDVLKRAATKKSRNDLIHAEEGEDRFVDLCAAGSPFARASLKSGKTESSSYFRRKEKMFRFLIRRMVKAQSFYWVVLCVVALNTLCVAMVHYNQPQRLTTALYFAEFVFLGLFLTEMSLKMYGLGPRSYFRSSFNCFDFGVIVGSIFEVVWAAIKPGTSFGISVLRALRLLRIFKVTKYWNSLRNLVVSLLNSMKSIISLLFLLFLFIVVFALLGMQLFGGQFNFQDETPTTNFDTFPAAILTVFQILTGEDWNAVMYHGIESQGGVSKGMFSSFYFIVLTLFGNYTLLNVFLAIAVDNLANAQELTKDEEEMEEAANQKLALQKAKEVAEVSPMSAANISIAARQQNSAKARSVWEQRASQLRLQNLRASCEALYSEMDPEERLRYASTRHVRPDMKTHMDRPLVVEPGRDGLRGPAGNKSKPEGTEATEGADPPRRHHRHRDRDKTSASTPAGGEQDRTDCPKAESTETGAREERARPRRSHSKEAPGADTQVRCERSRRHHRRGSPEEATEREPRRHRAHRHAQDSSKEGKEGTAPVLVPKGERRARHRGPRTGPRETENSEEPTRRHRAKHKVPPTLEPPEREVAEKESNVVEGDKETRNHQPKEPRCDLEAIAVTGVGSLHMLPSTCLQKVDEQPEDADNQRNVTRMGSQPSDPSTTVHVPVTLTGPPGEATVVPSANTDLEGQAEGKKEAEADDVLRRGPRPIVPYSSMFCLSPTNLLRRFCHYIVTMRYFEMVILVVIALSSIALAAEDPVRTDSFRNNALKYMDYIFTGVFTFEMVIKMIDLGLLLHPGAYFRDLWNILDFIVVSGALVAFAFSGSKGKDINTIKSLRVLRVLRPLKTIKRLPKLKAVFDCVVNSLKNVLNILIVYMLFMFIFAVIAVQLFKGKFFYCTDESKELERDCRGQYLDYEKEEVEAQPRQWKKYDFHYDNVLWALLTLFTVSTGEGWPMVLKHSVDATYEEQGPSPGFRMELSIFYVVYFVVFPFFFVNIFVALIIITFQEQGDKVMSECSLEKNERACIDFAISAKPLTRYMPQNKQSFQYKTWTFVVSPPFEYFIMAMIALNTVVLMMKFYDAPYEYELMLKCLNIVFTSMFSLECILKIIAFGVLNYFRDAWNVFDFVTVLGSITDILVTEIAETNNFINLSFLRLFRAARLIKLLRQGYTIRILLWTFVQSFKALPYVCLLIAMLFFIYAIIGMQVFGNIALDDGTSINRHNNFRTFLQALMLLFRSATGEAWHEIMLSCLGNRACDPHANASECGSDFAYFYFVSFIFLCSFLMLNLFVAVIMDNFEYLTRDSSILGPHHLDEFIRVWAEYDPAACGRISYNDMFEMLKHMSPPLGLGKKCPARVAYKRLVRMNMPISNEDMTVHFTSTLMALIRTALEIKLAPAGTKQHQCDAELRKEISSVWANLPQKTLDLLVPPHKPDEMTVGKVYAALMIFDFYKQNKTTRDQTHQAPGGLSQMGPVSLFHPLKATLEQTQPAVLRGARVFLRQKSATSLSNGGAIQTQESGIKESLSWGTQRTQDVLYEARAPLERGHSAEIPVGQPGALAVDVQMQNMTLRGPDGEPQPGLESQGRAASMPRLAAETQPAPNASPMKRSISTLAPRPHGTQLCNTVLDRPPPSQVSHHHHHRCHRRRDKKQRSLEKGPSLSVDTEGAPSTAAGSGLPHGEGSTGCRRERKQERGRSQERRQPSSSSSEKQRFYSCDRFGSREPPQPKPSLSSHPISPTAALEPGPHPQGSGSVNGSPLMSTSGASTPGRGGRRQLPQTPLTPRPSITYKTANSSPVHFAEGQSGLPAFSPGRLSRGLSEHNALLQKEPLSQPLASGSRIGSDPYLGQRLDSEASAHNLPEDTLTFEEAVATNSGRSSRTSYVSSLTSQSHPLRRVPNGYHCTLGLSTGVRARHSYHHPDQDHWC

SEQ ID NO: 29 - DNA, 7011 bp, Rattus norvegicus
  CDS (1)..(7011)
atggtccgcttcggggacgagctaggcggccgctatgggggcaccggcggcggggagcgggctcggggcggcggggccggcggggccggtggcccgggccaggggggtctgccgccgggccagcgggtcctgtacaagcagtccattgcgcaacgcgcacggaccatggccctgtacaaccccatcccagtcaagcagaactgcttcaccgtcaaccgctcgctcttcgtcttcagcgaggacaacgtcgtccgcaaatatgctaagcgcatcaccgaatggccgcccttcgaatatatgatcctggccaccatcatcgccaactgtattgtcctggccctggagcagcacctccctgatggggacaagactcccatgtctgaacgactggatgacacggaaccttacttcatcggcatcttttgcttcgaggcgggcatcaagatcatagctctgggcttcgtgttccacaaaggctcctacctccggaatggctggaacgtcatggacttcgtggtggtcctcacagagattcttgccacagctggaactgactttgatctgcgcaccctgagggctgtgcgtgtgcttaggcccctgaagttggtgtctggaattccaagcttgcaggtggtgctcaagtccatcatgaaggccatggtcccgctgctgcagatcgggctgctgctcttcttcgccatcctcatgttcgctatcatcggcctcgagttctatatgggcaaattccataaggcctgcttccccaacagcacagatgcagagcctgtgggtgactttccttgtggcaaggaggcccctgctcgtctgtgtgacagtgacaccgaatgccgggagtactggccaggacccaactttggcatcaccaattttgacaacatcctgtttgccatcttgaccgtgttccagtgtatcaccatggagggctggactgacatcctctacaatacaaatgatgcggccggcaacacgtggaactggttgtacttcatccccctcatcatcattggctccttcttcatgctcaacctggtgctcggtgtgctttcaggagagtttgccaaagagcgggagcgagtcgagaaccgccgtgccttcctgaagctccgcaggcagcagcagattgagcgagaactgaatgggtacttggagtggatcttcaaggcggaggaagtcatgttggcagaggaggacaagaacgcagaagagaagtcccctttggatgcagtgttgaagagagctgctaccaagaagagccgaaatgacctcatccatgcagaagagggggaggaccggtttgtagacctctgtgctgctgggtctccctttgctcgtgccagcctcaagagtgggaagacagagagctcatcgtacttccggaggaaggagaagatgttccggttccttatccgtcgtatggtgaaagcacagagcttctactgggtggtactgtgcgtggtggccctgaacacgttgtgtgtggccatggtacactataatcagcctcagcggcttaccactgcactgtactttgcagagtttgttttcctgggtctcttcctcacagagatgtccctgaagatgtacggtctagggcccagaagctacttccggtcttccttcaactgctttgactttggggtgattgtggggagtatctttgaagtagtctgggctgccatcaagccaggaacctccttcggaatcagtgtgctgcgggctctccgactgctgaggattttcaaagtcaccaagtattggaactccctgaggaacctggttgtttccctcctcaactccatgaagtccatcatcagccttctcttcctgcttttccttttcattgtggtcttcgctctgttggggatgcagctgtttgggggacagttcaactttcaagatgagactccaaccaccaattttgataccttcccagctgccatcctcactgtgtttcagattctgacaggagaggactggaatgcagtcatgtatcatgggattgagtcacaaggaggagtcagcaaaggcatgttttcatccttttacttcatcgtcctgacactgtttggaaactacaccctgttgaacgttttcttggccattgctgtggacaaccttgccaatgcccaggagttgaccaaggatgaagaggagatggaagaggcagccaatcagaagcttgctcttcagaaggccaaagaagtagctgaagtcagccccatgtctgctgccaacatctccattgctgccaggcagcagaactcggccaaggcgcgctcagtatgggagcagcgggccagtcagctaaggctccagaacctgcgtgccagctgtgaggcactgtacagtgagatggacccggaggagcgcctgcgttatgccagcacgcgccacgtgaggccagacatgaagacacacatggaccgacccctagtggtggaacctggtcgggatggcctgcggggacccgccgggaacaagtcaaagcctgagggcacggaggccaccgaaggtgcggatccaccacgccgacaccaccggcatcgtgatagggacaagacctcagcctcaacccctgctggaggcgaacaggacaggacagactgcccaaaggccgaaagcaccgagaccggggcccgggaggaacgtgcgcgccctcgtcgaagtcacagcaaggaggctccaggggctgacacacaagtgcgttgtgagcgcagtagacgtcaccaccggcgcggatccccggaggaggccactgaacgggaacctcggcgccaccgtgcccaccggcacgcacaggactcaagcaaggaaggcaaggagggcactgcaccggtgcttgtacccaagggcgagcgtcgcgcaagacatcgaggcccgcgtacgggcccccgtgagacagagaacagtgaggagcccacacgcaggcaccgtgcaaagcataaggtgccaccaacacttgagcccccagagagggaggttgcagagaaggagagcaacgtggtggaaggggataaggaaactcgaaatcaccagcccaaggaacctcgctgtgacctggaggccattgcggttacaggcgtgggctctctgcacatgctgcccagcacctgtctccagaaagtggacgaacagccagaggatgcagacaaccagcgtaatgtcacccggatgggcagtcagccctcagaccccagcaccactgtgcatgtcccagtgacactgacaggccctcccggggaggccactgtagttcccagtgctaacacggacctggaaggccaagcggagggcaagaaggaggcagaggctgacgatgtgctgagaagaggccccaggcccatcgttccctacagttccatgttctgcctcagccccaccaacctactccgtcgcttctgccattacattgtgaccatgcggtactttgagatggtgattcttgtggtcatcgccttgagcagcattgccctggctgctgaggatcccgtgcggaccgactcattccggaacaatgctctgaagtacatggactacatctttacaggagtcttcacctttgagatggtcataaagatgatagacttgggcctgctgctgcaccctggggcctacttccgggacctgtggaacattctggacttcattgttgtcagtggagccctggtggcatttgcattctcgagcttcatgggaggatccaaagggaaagacatcaataccatcaagtctctgagagtcctgcgagtcctgcggcccctcaagaccatcaagcggctgcctaaactcaaggctgtgtttgactgtgtggtgaactctctgaagaatgtcttgaacatcctgatcgtctacatgctcttcatgtttatatttgccgtcatcgccgtccaactcttcaaagggaagttcttttactgcactgatgagtccaaggagctggagcgggactgcaggggtcagtatttggattatgagaaggaagaggtagaagcccagccaaggcagtggaagaaatatgacttccactatgacaatgtgctctgggccttgctgactctgtttacggtgtccacaggagaggggtggcccatggtgctgaaacactctgtggacgccacctatgaggagcaggggccaagccccgggtttcggatggagctttccatcttctatgtggtctactttgtggtcttcccttttttctttgtcaacatctttgtggccttgatcatcatcaccttccaggagcagggggacaaggtgatgtctgagtgcagtctggaaaagaatgagagggcttgcattgactttgccatcagcgccaaacccctgacacggtacatgcctcagaacaagcagtcgttccagtataagacatggacatttgtggtctctccaccctttgagtacttcattatggccatgatagccctcaacacagtggtgctgatgatgaagttctacgatgccccttatgagtacgagctgatgctgaagtgcttgaacatcgtcttcacatccatgttctctctggagtgcatcctgaagatcatcgccttcggggtgttgaactacttcagagatgcctggaacgtctttgactttgtcactgttttgggaagtattactgatattttagtaacggagattgcgaacaacttcatcaacttgagcttccttcgcctcttccgggcagcacggctgatcaagctctgtcgccagggctacaccatccgcatcttgttatggacctttgtccagtcctttaaggcgctgccctacgtgtgcctcctcattgccatgctgttcttcatctacgccatcatcggcatgcaggtttttggaaacattgcccttgatgatggcaccagcatcaaccgacacaacaacttccggacatttctgcaagccttaatgctgttgttcaggagtgccactggggaggcctggcacgaaatcatgctgtcttgcctgggcaaccgggcctgcgacccacatgccaacgccagcgaatgcgggagcgactttgcctatttttattttgtctccttcatcttcctctgttcctttctgatgctgaacctctttgttgctgtgatcatggacaatttcgaatacctcacgcgggattcttccatcctagggccgcaccacctcgatgaattcattcgcgtctgggctgaatacgacccagctgcgtgtgggcgcatcagttacaatgacatgtttgagatgctgaaacacatgtccccacctctgggtttggggaagaaatgcccggctcgagttgcatacaagcgcctggttcgaatgaacatgcccatatccaatgaggacatgacggtacactttacatccacactgatggccctcatccggacggcactggagatcaagcttgccccagcggggacaaaacagcaccaatgtgatgctgagctgaggaaggagatctcttctgtgtgggctaatctgccccagaagactctggacttactggtgccaccccacaaacctgacgagatgacagtggggaaggtctatgcggctctcatgatatttgacttctacaaacagaacaaaaccaccagagatcagactcaccaagctcctggaggcctgtcccagatgggtcctgtttccctattccatcctctgaaggccaccctggagcagacacagcccgctgtgctccgaggagctcgggttttccttcgacaaaagagtgcaacttccctcagcaatgggggcgccatacaaacccaggaaagtggcatcaaggagtccctgtcctggggcacgcagaggacccaggacgtactttatgaggccagagcacctctagaacgtggccattctgcagagatccctgtggggcagccaggagcactggctgtagatgtccagatgcagaacatgacattgagaggaccggatggggagccccagcctggcctggagagccaaggccgagcggcctctatgccacgcctggcggcagaaacacagccggcccctaatgccagccccatgaagcgctccatctccacactggctccacgcccgcatgggactcagctttgcaacacagtcctggaccggccacctcctagccaggtgtcccatcaccaccaccaccgctgccaccggcgcagggacaagaagcagaggtccctggaaaaggggcccagcctgtctgttgacacagaaggtgcaccaagtactgctgcaggatctggcctgccccatggagaagggtccacaggctgccggcgggagcgtaagcaagagcgaggccggtcccaggagcggaggcagccctcctcctcttcttcagagaagcagcgcttctattcctgtgaccgctttgggagccgggagcccccacaacctaagccctccctcagtagccaccccatatcgccaacagcggcactagagccaggaccccacccgcagggcagtggttccgttaatgggagccccttgatgtcaacatctggtgctagcacgccgggccgaggtgggcggaggcagctcccccagactcccctgaccccacgccccagcatcacctacaagacggccaattcctcgcctgtccactttgctgagggtcagagtggccttccagccttctcccctggccgtctcagccgcggcctttctgaacacaatgccctgctccagaaagagcccctgagccagcctctagcttctggctcccgcattggctctgacccttacctagggcagcgtctggacagtgaggcctctgcccacaacctgcctgaggatacactcacctttgaagaggccgtggccaccaactctggccgctcctccaggacttcctatgtgtcctccctcacttcccaatcccaccctctccgccgtgtacccaatggctaccactgcactttgggactcagcaccggcgtccgggcgcggcacagctaccaccacccagaccaggatcactggtgctag

SEQ ID NO: 30 - protein, 2336 aa, Rattus norvegicus
MVRFGDELGGRYGGTGGGERARGGGAGGAGGPGQGGLPPGQRVLYKQSIAQRARTMALYNPIPVKQNCFTVNRSLFVFSEDNVVRKYAKRITEWPPFEYMILATIIANCIVLALEQHLPDGDKTPMSERLDDTEPYFIGIFCFEAGIKIIALGFVFHKGSYLRNGWNVMDFVVVLTEILATAGTDFDLRTLRAVRVLRPLKLVSGIPSLQVVLKSIMKAMVPLLQIGLLLFFAILMFAIIGLEFYMGKFHKACFPNSTDAEPVGDFPCGKEAPARLCDSDTECREYWPGPNFGITNFDNILFAILTVFQCITMEGWTDILYNTNDAAGNTWNWLYFIPLIIIGSFFMLNLVLGVLSGEFAKERERVENRRAFLKLRRQQQIERELNGYLEWIFKAEEVMLAEEDKNAEEKSPLDAVLKRAATKKSRNDLIHAEEGEDRFVDLCAAGSPFARASLKSGKTESSSYFRRKEKMFRFLIRRMVKAQSFYWVVLCVVALNTLCVAMVHYNQPQRLTTALYFAEFVFLGLFLTEMSLKMYGLGPRSYFRSSFNCFDFGVIVGSIFEVVWAAIKPGTSFGISVLRALRLLRIFKVTKYWNSLRNLVVSLLNSMKSIISLLFLLFLFIVVFALLGMQLFGGQFNFQDETPTTNFDTFPAAILTVFQILTGEDWNAVMYHGIESQGGVSKGMFSSFYFIVLTLFGNYTLLNVFLAIAVDNLANAQELTKDEEEMEEAANQKLALQKAKEVAEVSPMSAANISIAARQQNSAKARSVWEQRASQLRLQNLRASCEALYSEMDPEERLRYASTRHVRPDMKTHMDRPLVVEPGRDGLRGPAGNKSKPEGTEATEGADPPRRHHRHRDRDKTSASTPAGGEQDRTDCPKAESTETGAREERARPRRSHSKEAPGADTQVRCERSRRHHRRGSPEEATEREPRRHRAHRHAQDSSKEGKEGTAPVLVPKGERRARHRGPRTGPRETENSEEPTRRHRAKHKVPPTLEPPEREVAEKESNVVEGDKETRNHQPKEPRCDLEAIAVTGVGSLHMLPSTCLQKVDEQPEDADNQRNVTRMGSQPSDPSTTVHVPVTLTGPPGEATVVPSANTDLEGQAEGKKEAEADDVLRRGPRPIVPYSSMFCLSPTNLLRRFCHYIVTMRYFEMVILVVIALSSIALAAEDPVRTDSFRNNALKYMDYIFTGVFTFEMVIKMIDLGLLLHPGAYFRDLWNILDFIVVSGALVAFAFSSFMGGSKGKDINTIKSLRVLRVLRPLKTIKRLPKLKAVFDCVVNSLKNVLNILIVYMLFMFIFAVIAVQLFKGKFFYCTDESKELERDCRGQYLDYEKEEVEAQPRQWKKYDFHYDNVLWALLTLFTVSTGEGWPMVLKHSVDATYEEQGPSPGFRMELSIFYVVYFVVFPFFFVNIFVALIIITFQEQGDKVMSECSLEKNERACIDFAISAKPLTRYMPQNKQSFQYKTWTFVVSPPFEYFIMAMIALNTVVLMMKFYDAPYEYELMLKCLNIVFTSMFSLECILKIIAFGVLNYFRDAWNVFDFVTVLGSITDILVTEIANNFINLSFLRLFRAARLIKLCRQGYTIRILLWTFVQSFKALPYVCLLIAMLFFIYAIIGMQVFGNIALDDGTSINRHNNFRTFLQALMLLFRSATGEAWHEIMLSCLGNRACDPHANASECGSDFAYFYFVSFIFLCSFLMLNLFVAVIMDNFEYLTRDSSILGPHHLDEFIRVWAEYDPAACGRISYNDMFEMLKHMSPPLGLGKKCPARVAYKRLVRMNMPISNEDMTVHFTSTLMALIRTALEIKLAPAGTKQHQCDAELRKEISSVWANLPQKTLDLLVPPHKPDEMTVGKVYAALMIFDFYKQNKTTRDQTHQAPGGLSQMGPVSLFHPLKATLEQTQPAVLRGARVFLRQKSATSLSNGGAIQTQESGIKESLSWGTQRTQDVLYEARAPLERGHSAEIPVGQPGALAVDVQMQNMTLRGPDGEPQPGLESQGRAASMPRLAAETQPAPNASPMKRSISTLAPRPHGTQLCNTVLDRPPPSQVSHHHHHRCHRRRDKKQRSLEKGPSLSVDTEGAPSTAAGSGLPHGEGSTGCRRERKQERGRSQERRQPSSSSSEKQRFYSCDRFGSREPPQPKPSLSSHPISPTAALEPGPHPQGSGSVNGSPLMSTSGASTPGRGGRRQLPQTPLTPRPSITYKTANSSPVHFAEGQSGLPAFSPGRLSRGLSEHNALLQKEPLSQPLASGSRIGSDPYLGQRLDSEASAHNLPEDTLTFEEAVATNSGRSSRTSYVSSLTSQSHPLRRVPNGYHCTLGLSTGVRARHSYHHPDQDHWC

SEQ ID NO: 31 - DNA, 7364 bp, Homo sapiens
  CDS (146)..(7165)
gcggcggcggctgcggcggtggggccgggcgaggtccgctgcggtcccggcggctccgtggctgctccgctctgagcgcctggcgcgccccgcgccctccctgccggggccgctgggccggggatgcacgcggggcccgggagccatggtccgcttcggggacgagctgggcggccgctatggaggccccggcggcggagagcgggcccggggcggcggggccggcggggcggggggcccgggtcccggggggctgcagcccggccagcgggtcctctacaagcaatcgatcgcgcagcgcgcgcggaccatggcgctgtacaaccccatcccggtcaagcagaactgcttcaccgtcaaccgctcgctcttcgtcttcagcgaggacaacgtcgtccgcaaatacgcgaagcgcatcaccgagtggcctccattcgagtatatgatcctggccaccatcatcgccaactgcatcgtgctggccctggagcagcacctccctgatggggacaaaacgcccatgtccgagcggctggacgacacggagccctatttcatcgggatcttttgcttcgaggcagggatcaaaatcatcgctctgggctttgtcttccacaagggctcttacctgcggaacggctggaacgtcatggacttcgtggtcgtcctcacagggatccttgccacggctggaactgacttcgacctgcgaacactgagggctgtgcgtgtgctgaggcccctgaagctggtgtctgggattccaagtttgcaggtggtgctcaagtccatcatgaaggccatggttccactcctgcagattgggctgcttctcttctttgccatcctcatgtttgccatcattggcctggagttctacatgggcaagttccacaaggcctgtttccccaacagcacagatgcggagcccgtgggtgacttcccctgtggcaaggaggccccagcccggctgtgcgagggcgacactgagtgccgggagtactggccaggacccaactttggcatcaccaactttgacaatatcctgtttgccatcttgacggtgttccagtgcatcaccatggagggctggactgacatcctctataatacaaacgatgcggccggcaacacctggaactggctctacttcatccctctcatcatcatcggctccttcttcatgctcaacctggtgctgggcgtgctctcgggggagtttgccaaggagcgagagagggtggagaaccgccgcgccttcctgaagctgcgccggcagcagcagatcgagcgagagctcaacgggtacctggagtggatcttcaaggcggaggaagtcatgctggccgaggaggacaggaatgcagaggagaagtcccctttggacgtgctgaagagagcggccaccaagaagagcagaaatgacctgatccacgcagaggagggagaggaccggtttgcagatctctgtgctgttggatcccccttcgcccgcgccagcctcaagagcgggaagacagagagctcgtcatacttccggaggaaggagaagatgttccggttttttatccggcgcatggtgaaggctcagagcttctactgggtggtgctgtgcgtggtggccctgaacacactgtgtgtggccatggtgcattacaaccagccgcggcggcttaccacgaccctgtattttgcagagtttgttttcctgggtctcttcctcacagagatgtccctgaagatgtatggcctggggcccagaagctacttccggtcctccttcaactgcttcgactttggggtcatcgtggggagcgtctttgaagtggtctgggcggccatcaagccgggaagctcctttgggatcagtgtgctgcgggccctccgcctgctgaggatcttcaaagtcacgaagtactggagctccctgcggaacctggtggtgtccctgctgaactccatgaagtccatcatcagcctgctcttcttgctcttcctgttcattgtggtcttcgccctgctggggatgcagctgtttgggggacagttcaacttccaggatgagactcccacaaccaacttcgacaccttccctgccgccatcctcactgtcttccagatcctgacgggagaggactggaatgcagtgatgtatcacgggatcgaatcgcaaggcggcgtcagcaaaggcatgttctcgtccttttacttcattgtcctgacactgttcggaaactacactctgctgaatgtctttctggccatcgctgtggacaacctggccaacgcccaagagctgaccaaggatgaagaggagatggaagaagcagccaatcagaagcttgctctgcaaaaggccaaagaagtggctgaagtcagccccatgtctgccgcgaacatctccatcgccgccaggcagcagaactcggccaaggcgcgctcggtgtgggagcagcgggccagccagctacggctgcagaacctgcgggccagctgcgaggcgctgtacagcgagatggaccccgaggagcggctgcgcttcgccactacgcgccacctgcggcccgacatgaagacgcacctggaccggccgctggtggtggagctgggccgcgacggcgcgcgggggcccgtgggaggcaaagcccgacctgaggctgcggaggcccccgagggcgtcgaccctccgcgcaggcaccaccggcaccgcgacaaggacaagacccccgcggcgggggaccaggaccgagcagaggccccgaaggcggagagcggggagcccggtgcccgggaggagcggccgcggccgcaccgcagccacagcaaggaggccgcggggcccccggaggcgcggagcgagcgcggccgaggcccaggccccgagggcggccggcggcaccaccggcgcggctccccggaggaggcggccgagcgggagccccgacgccaccgcgcgcaccggcaccaggatccgagcaaggagtgcgccggcgccaagggcgagcggcgcgcgcggcaccgcggcggcccccgagcggggccccgggaggcggagagcggggaggagccggcgcggcggcaccgggcccggcacaaggcgcagcctgctcacgaggctgtggagaaggagaccacggagaaggaggccacggagaaggaggctgagatagtggaagccgacaaggaaaaggagctccggaaccaccagccccgggagccacactgtgacctggagaccagtgggactgtgactgtgggtcccatgcacacactgcccagcacctgtctccagaaggtggaggaacagccagaggatgcagacaatcagcggaacgtcactcgcatgggcagtcagcccccagacccgaacactattgtacatatcccagtgatgctgacgggccctcttggggaagccacggtcgttcccagtggtaacgtggacctggaaagccaagcagaggggaagaaggaggtggaagcggatgacgtgatgaggagcggcccccggcctatcgtcccatacagctccatgttctgtttaagccccaccaacctgctccgccgcttctgccactacatcgtgaccatgaggtacttcgaggtggtcattctcgtggtcatcgccttgagcagcatcgccctggctgctgaggacccagtgcgcacagactcgcccaggaacaacgctctgaaatacctggattacattttcactggtgtctttacctttgagatggtgataaagatgatcgacttgggactgctgcttcaccctggagcctatttccgggacttgtggaacattctggacttcattgtggtcagtggcgccctggtggcgtttgctttctcaggatccaaagggaaagacatcaataccatcaagtctctgagagtccttcgtgtcctgcggcccctcaagaccatcaaacggctgcccaagctcaaggctgtgtttgactgtgtggtgaactccctgaagaatgtcctcaacatcttgattgtctacatgctcttcatgttcatatttgccgtcattgcggtgcagctcttcaaagggaagtttttctactgcacagatgaatccaaggagctggagagggactgcaggggtcagtatttggattatgagaaggaggaagtggaagctcagcccaggcagtggaagaaatacgactttcactacgacaatgtgctctgggctctgctgacgctgttcacagtgtccacgggagaaggctggcccatggtgctgaaacactccgtggatgccacctatgaggagcagggtccaagccctgggtaccgcatggagctgtccatcttctacgtggtctactttgtggtctttcccttcttcttcgtcaacatctttgtggctttgatcatcatcaccttccaggagcagggggacaaggtgatgtctgaatgcagcctggagaagaacgagagggcttgcattgacttcgccatcagcgccaaacccctgacacggtacatgccccaaaaccggcagtcgttccagtataagacgtggacatttgtggtctccccgccctttgaatacttcatcatggccatgatagccctcaacactgtggtgctgatgatgaagttctatgatgcaccctatgagtacgagctgatgctgaaatgcctgaacatcgtgttcacatccatgttctccatggaatgcgtgctgaagatcatcgcctttggggtgctgaactatttcagagatgcctggaatgtctttgactttgtcactgtgttgggaagtattactgatattttagtaacagagattgcggaaacgaacaatttcatcaacctcagcttcctccgcctctttcgagctgcgcggctgatcaagctgctccgccagggctacaccatccgcatcctgctgtggacctttgtccagtccttcaaggccctgccctacgtgtgtctgctcattgccatgctgttcttcatctacgccatcatcggcatgcaggtgtttgggaatattgccctggatgatgacaccagcatcaaccgccacaacaacttccggacgtttttgcaagccctgatgctgctgttcaggagcgccacgggggaggcctggcacgagatcatgctgtcctgcctgagcaaccaggcctgtgatgagcaggccaatgccaccgagtgtggaagtgactttgcctacttctacttcgtctccttcatcttcctgtgctcctttctgatgttgaacctctttgtggctgtgatcatggacaattttgagtacctcacgcgggactcttccatcctaggtcctcaccacttggatgagttcatccgggtctgggctgaatacgacccggctgcgtgtgggcgcatcagttacaatgacatgtttgagatgctgaaacacatgtccccgcctctggggctggggaagaaatgccctgctcgagttgcttacaagcgcctggttcgcatgaacatgcccatctccaacgaggacatgactgttcacttcacgtccacgctgatggccctcatccggacggcactggagatcaagctggccccagctgggacaaagcagcatcagtgtgacgcggagttgaggaaggagatttccgttgtgtgggccaatctgccccagaagactttggacttgctggtaccaccccataagcctgatgagatgacagtggggaaggtttatgcagctctgatgatatttgacttctacaagcagaacaaaaccaccagagaccagatgcagcaggctcctggaggcctctcccagatgggtcctgtgtccctgttccaccctctgaaggccaccctggagcagacacagccggctgtgctccgaggagcccgggttttccttcgacagaagagttccacctccctcagcaatggcggggccatacaaaaccaagagagtggcatcaaagagtctgtctcctggggcactcaaaggacccaggatgcaccccatgaggccaggccacccctggagcgtggccactccacagagatccctgtggggcggtcaggagcactggctgtggacgttcagatgcagagcataacccggaggggccctgatggggagccccagcctgggctggagagccagggtcgagcggcctccatgccccgccttgcggccgagactcagcccgtcacagatgccagccccatgaagcgctccatctccacgctggcccagcggccccgtgggactcatctttgcagcaccaccccggaccgcccaccccctagccaggcgtcgtcgcaccaccaccaccaccgctgccaccgccgcagggacaggaagcagaggtccctggagaaggggcccagcctgtctgccgatatggatggcgcaccaagcagtgctgtggggccggggctgcccccgggagaggggcctacaggctgccggcgggaacgagagcgccggcaggagcggggccggtcccaggagcggaggcagccctcatcctcctcctcggagaagcagcgcttctactcctgcgaccgctttgggggccgtgagcccccgaagcccaagccctccctcagcagccacccaacgtcgccaacagctggccaggagccgggaccccacccacagggcagtggttccgtgaatgggagccccttgctgtcaacatctggtgctagcacccccggccgcggtgggcggaggcagctcccccagacgcccctgactccccgccccagcatcacctacaagacggccaactcctcacccatccacttcgccggggctcagaccagcctccctgccttctccccaggccggctcagccgtgggctttccgaacacaacgccctgctgcagagagaccccctcagccagcccctggcccctggctctcgaattggctctgacccttacctggggcagcgtctggacagtgaggcctctgtccacgccctgcctgaggacacgctcactttcgaggaggctgtggccaccaactcgggccgctcctccaggacttcctacgtgtcctccctgacctcccagtctcaccctctccgccgcgtgcccaacggttaccactgcaccctgggactcagctcgggtggccgagcacggcacagctaccaccaccctgaccaagaccactggtgctagctgcaccgtgaccgctcagacgcctgcatgcagcaggcgtgtgttccagtggatgagttttatcatccacacggggcagtcggccctcgggggaggccttgcccaccttggtgaggctcctgtggcccctccctccccctcctcccctcttttactctagacgacgaataaagccctgttgcttgagtgtacgtaccgc

SEQ ID NO: 32 - protein, 2339 aa, Homo sapiens [sequence omitted]

SEQ ID NO: 33 - DNA, 7177 bp, Homo sapiens
  CDS (146)..(6859)
gcggcggcggctgcggcggtggggccgggcgaggtccgctgcggtcccggcggctccgtggctgctccgctctgagcgcctggcgcgccccgcgccctccctgccggggccgctgggccggggatgcacgcggggcccgggagccatggtccgcttcggggacgagctgggcggccgctatggaggccccggcggcggagagcgggcccggggcggcggggccggcggggcggggggcccgggtcccggggggctgcagcccggccagcgggtcctctacaagcaatcgatcgcgcagcgcgcgcggaccatggcgctgtacaaccccatcccggtcaagcagaactgcttcaccgtcaaccgctcgctcttcgtcttcagcgaggacaacgtcgtccgcaaatacgcgaagcgcatcaccgagtggcctccattcgagtatatgatcctggccaccatcatcgccaactgcatcgtgctggccctggagcagcacctccctgatggggacaaaacgcccatgtccgagcggctggacgacacggagccctatttcatcgggatcttttgcttcgaggcagggatcaaaatcatcgctctgggctttgtcttccacaagggctcttacctgcggaacggctggaacgtcatggacttcgtggtcgtcctcacagggatccttgccacggctggaactgacttcgacctgcgaacactgagggctgtgcgtgtgctgaggcccctgaagctggtgtctgggattccaagtttgcaggtggtgctcaagtccatcatgaaggccatggttccactcctgcagattgggctgcttctcttctttgccatcctcatgtttgccatcattggcctggagttctacatgggcaagttccacaaggcctgtttccccaacagcacagatgcggagcccgtgggtgacttcccctgtggcaaggaggccccagcccggctgtgcgagggcgacactgagtgccgggagtactggccaggacccaactttggcatcaccaactttgacaatatcctgtttgccatcttgacggtgttccagtgcatcaccatggagggctggactgacatcctctataatacaaacgatgcggccggcaacacctggaactggctctacttcatccctctcatcatcatcggctccttcttcatgctcaacctggtgctgggcgtgctctcgggggagtttgccaaggagcgagagagggtggagaaccgccgcgccttcctgaagctgcgccggcagcagcagatcgagcgagagctcaacgggtacctggagtggatcttcaaggcggaggaagtcatgctggccgaggaggacaggaatgcagaggagaagtcccctttggacgtgctgaagagagcggccaccaagaagagcagaaatgacctgatccacgcagaggagggagaggaccggtttgcagatctctgtgctgttggatcccccttcgcccgcgccagcctcaagagcgggaagacagagagctcgtcatacttccggaggaaggagaagatgttccggttttttatccggcgcatggtgaaggctcagagcttctactgggtggtgctgtgcgtggtggccctgaacacactgtgtgtggccatggtgcattacaaccagccgcggcggcttaccacgaccctgtattttgcagagtttgttttcctgggtctcttcctcacagagatgtccctgaagatgtatggcctggggcccagaagctacttccggtcctccttcaactgcttcgactttggggtcatcgtggggagcgtctttgaagtggtctgggcggccatcaagccgggaagctcctttgggatcagtgtgctgcgggccctccgcctgctgaggatcttcaaagtcacgaagtactggagctccctgcggaacctggtggtgtccctgctgaactccatgaagtccatcatcagcctgctcttcttgctcttcctgttcattgtggtcttcgccctgctggggatgcagctgtttgggggacagttcaacttccaggatgagactcccacaaccaacttcgacaccttccctgccgccatcctcactgtcttccagatcctgacgggagaggactggaatgcagtgatgtatcacgggatcgaatcgcaaggcggcgtcagcaaaggcatgttctcgtccttttacttcattgtcctgacactgttcggaaactacactctgctgaatgtctttctggccatcgctgtggacaacctggccaacgcccaagagctgaccaaggatgaagaggagatggaagaagcagccaatcagaagcttgctctgcaaaaggccaaagaagtggctgaagtcagccccatgtctgccgcgaacatctccatcgccgccaggcagcagaactcggccaaggcgcgctcggtgtgggagcagcgggccagccagctacggctgcagaacctgcgggccagctgcgaggcgctgtacagcgagatggaccccgaggagcggctgcgcttcgccactacgcgccacctgcggcccgacatgaagacgcacctggaccggccgctggtggtggagctgggccgcgacggcgcgcgggggcccgtgggaggcaaagcccgacctgaggctgcggaggcccccgagggcgtcgaccctccgcgcaggcaccaccggcaccgcgacaaggacaagacccccgcggcgggggaccaggaccgagcagaggccccgaaggcggagagcggggagcccggtgcccgggaggagcggccgcggccgcaccgcagccacagcaaggaggccgcggggcccccggaggcgcggagcgagcgcggccgaggcccaggccccgagggcggccggcggcaccaccggcgcggctccccggaggaggcggccgagcgggagccccgacgccaccgcgcgcaccggcaccaggatccgagcaaggagtgcgccggcgccaagggcgagcggcgcgcgcggcaccgcggcggcccccgagcggggccccgggaggcggagagcggggaggagccggcgcggcggcaccgggcccggcacaaggcgcagcctgctcacgaggctgtggagaaggagaccacggagaaggaggccacggagaaggaggctgagatagtggaagccgacaaggaaaaggagctccggaaccaccagccccgggagccacactgtgacctggagaccagtgggactgtgactgtgggtcccatgcacacactgcccagcacctgtctccagaaggtggaggaacagccagaggatgcagacaatcagcggaacgtcactcgcatgggcagtcagcccccagacccgaacactattgtacatatcccagtgatgctgacgggccctcttggggaagccacggtcgttcccagtggtaacgtggacctggaaagccaagcagaggggaagaaggaggtggaagcggatgacgtgatgaggagcggcccccggcctatcgtcccatacagctccatgttctgtttaagccccaccaacctgctccgccgcttctgccactacatcgtgaccatgaggtacttcgaggtggtcattctcgtggtcatcgccttgagcagcatcgccctggctgctgaggacccagtgcgcacagactcgcccaggaacaacgctctgaaatacctggattacattttcactggtgtctttacctttgagatggtgataaagatgatcgacttgggactgctgcttcaccctggagcctatttccgggacttgtggaacattctggacttcattgtggtcagtggcgccctggtggcgtttgctttctcaggatccaaagggaaagacatcaataccatcaagtctctgagagtccttcgtgtcctgcggcccctcaagaccatcaaacggctgcccaagctcaaggctgtgtttgactgtgtggtgaactccctgaagaatgtcctcaacatcttgattgtctacatgctcttcatgttcatatttgccgtcattgcggtgcagctcttcaaagggaagtttttctactgcacagatgaatccaaggagctggagagggactgcaggggtcagtatttggattatgagaaggaggaagtggaagctcagcccaggcagtggaagaaatacgactttcactacgacaatgtgctctgggctctgctgacgctgttcacagtgtccacgggagaaggctggcccatggtgctgaaacactccgtggatgccacctatgaggagcagggtccaagccctgggtaccgcatggagctgtccatcttctacgtggtctactttgtggtctttcccttcttcttcgtcaacatctttgtggctttgatcatcatcaccttccaggagcagggggacaaggtgatgtctgaatgcagcctggagaagaacgagagggcttgcattgacttcgccatcagcgccaaacccctgacacggtacatgccccaaaaccggcagtcgttccagtataagacgtggacatttgtggtctccccgccctttgaatacttcatcatggccatgatagccctcaacactgtggtgctgatgatgaagttctatgatgcaccctatgagtacgagctgatgctgaaatgcctgaacatcgtgttcacatccatgttctccatggaatgcgtgctgaagatcatcgcctttggggtgctgaactatttcagagatgcctggaatgtctttgactttgtcactgtgttgggaagtattactgatattttagtaacagagattgcggaaacgaacaatttcatcaacctcagcttcctccgcctctttcgagctgcgcggctgatcaagctgctccgccagggctacaccatccgcatcctgctgtggacctttgtccagtccttcaaggccctgccctacgtgtgtctgctcattgccatgctgttcttcatctacgccatcatcggcatgcaggtgtttgggaatattgccctggatgatgacaccagcatcaaccgccacaacaacttccggacgtttttgcaagccctgatgctgctgttcaggagcgccacgggggaggcctggcacgagatcatgctgtcctgcctgagcaaccaggcctgtgatgagcaggccaatgccaccgagtgtggaagtgactttgcctacttctacttcgtctccttcatcttcctgtgctcctttctgatgttgaacctctttgtggctgtgatcatggacaattttgagtacctcacgcgggactcttccatcctaggtcctcaccacttggatgagttcatccgggtctgggctgaatacgacccggctgcgtgtgggcgcatcagttacaatgacatgtttgagatgctgaaacacatgtccccgcctctggggctggggaagaaatgccctgctcgagttgcttacaagcgcctggttcgcatgaacatgcccatctccaacgaggacatgactgttcacttcacgtccacgctgatggccctcatccggacggcactggagatcaagctggccccagctgggacaaagcagcatcagtgtgacgcggagttgaggaaggagatttccgttgtgtgggccaatctgccccagaagactttggacttgctggtaccaccccataagcctgatgagatgacagtggggaaggtttatgcagctctgatgatatttgacttctacaagcagaacaaaaccaccagagaccagatgcagcaggctcctggaggcctctcccagatgggtcctgtgtccctgttccaccctctgaaggccaccctggagcagacacagccggctgtgctccgaggagcccgggttttccttcgacagaagagttccacctccctcagcaatggcggggccatacaaaaccaagagagtggcatcaaagagtctgtctcctggggcactcaaaggacccaggatgcaccccatgaggccaggccacccctggagcgtggccactccacagagatccctgtggggcggtcaggagcactggctgtggacgttcagatgcagagcataacccggaggggccctgatggggagccccagcctgggctggagagccagggtcgagcggcctccatgccccgccttgcggccgagactcagcccgtcacagatgccagccccatgaagcgctccatctccacgctggcccagcggccccgtgggactcatctttgcagcaccaccccggaccgcccaccccctagccaggcgtcgtcgcaccaccaccaccaccgctgccaccgccgcagggacaggaagcagaggtccctggagaaggggcccagcctgtctgccgatatggatggcgcaccaagcagtgctgtggggccggggctgcccccgggagaggggcctacaggctgccggcgggaacgagagcgccggcaggagcggggccggtcccaggagcggaggcagccctcatcctcctcctcggagaagcagcgcttctactcctgcgaccgctttgggggccgtgagcccccgaagcccaagccctccctcagcagccacccaacgtcgccaacagctggccaggagccgggaccccacccacaggccggctcagccgtgggctttccgaacacaacgccctgctgcagagagaccccctcagccagcccctggcccctggctctcgaattggctctgacccttacctggggcagcgtctggacagtgaggcctctgtccacgccctgcctgaggacacgctcactttcgaggaggctgtggccaccaactcgggccgctcctccaggacttcctacgtgtcctccctgacctcccagtctcaccctctccgccgcgtgcccaacggttaccactgcaccctgggactcagctcgggtggccgagcacggcacagctaccaccaccctgaccaagaccactggtgctagctgcaccgtgaccgctcagacgcctgcatgcagcaggcgtgtgttccagtggatgagttttatcatccacacggggcagtcggccctcgggggaggccttgcccaccttggtgaggctcctgtggcccctccctccccctcctcccctcttttactctagacgacgaataaagccctgttgcttgagtgtacgtaccgc

SEQ ID NO: 34 - protein, 2237 aa, Homo sapiens [sequence omitted]

SEQ ID NO: 35 - DNA, 6176 bp, Homo sapiens
  exon (1)..(17): Exon 41, continues 5′ to nt 1
tacgacccggctgcgtggtaagtgagccgtggtgctctgtggtccttgggggtggtccatgcccatagatcttgggatgggcctggtggcttcagacatgtttcaagtgagcaccccctcaggttttggtttggggcctagggatctgtcctgtttcgtgtagagcagcagtgatgtcccctccgagcagcacctcccttttccatctcgtgtgccttcgcgcagtggcccggactgcataaagtgataagggtcttttgagaccttgagttcagttccttgggaggtaccaggagtcatggaaggttttcaaggggcttgtgagactcaggtcttctgctgaaatttactgtcttctttcaaggcctgtcatgtgagggctcatggagggcttgtgggagggtgtgtggagacagcacagccatagcgtccttgtgagactcgatgtagggttttaggacattctccctaaggggctcaggagtctccctctgctctttcttttggggacaggggccaacatgcacctctgtgtccccagcccaggctgcatatagtgtgggtgcttggtgaacttttttgacagatgagctaccaggaaggctgtagtgtgtttaagacccttgtctatcatctgtcatcgtgggaaaggtttacttgtttcaaaggcgaggggtggtgcctttccagacagaggaggagtcgcatgaagggcatggtttgggtcagaaggtgaagctgtttgagatcaggatccatgggggtcccgtacagcatggcatctcagcagggctgagagctcattcgggaaggaagacaggtcaggcagcatggcttctccatctttctgtttgtccattccttccatcactcgttcctcatttacttgctcgctcactcactcaagcaatgtctactgtgggctgctccctgcaaggccactaggcctgctagcacctcactcacagcgtgtgcccgggaaatgatcgtggatgagcagctctcagtgagtgcgtagacatccaccactccccgtgccctagggggatgagggaacygtgggtgccaccgaggcctgccttctctctctgagctccttagcctgcccaggccccctcgcactcctctgcccccagtgcccttactgggggagggccggcaggacgtggtcagacccctcatggtgaggtcctactgcccacagagctcctgggttctgcctggttttgcttaaactgcagctcagcagagggagccctccacagacacaaaaggcctgggagcccgactagacttccccaccctcctttccactcagcctcccsggagcacccccaccttcctagtgaggatccagagggtgctgcacgggcccttcccagtctggacaaggtccagagccactggcagcatcgggggccctgggatggggcctctcctgtccaggctcttccaggcacacagcatctctgtgggtgtttttcatttgagtcggaaatatacagtttttacgcagtgcaaaacagacgtagagattgtctgtgtttcacagtgtatatgacaatttccgtggcttgcgagaaccctgtcatgtccgatgcagcaatatgtttgccctgtgatacttacttcatgctgggagggacaggctcaggcacattcacacaggacctgtggtcaagtaagcccagtcgacctgggggagactgaccacattgaagacctagaccttgttaggcaaagggcaggagagactccagccgaggcaggatttaggtgaattctaggcaggtcaccctgaatgtgtggtttagagagtcgctgctttttctgggtgtattttttctrgaggagactgtgttgcagaaaacagagatactttatagcatgtagttagtactraagggcaaaggcagatcaacaaaggcctgtgagaggatgacaacattccccaatctttatgagctacgcccgggccttccacagtcagccgccggcaagagcagaggcaggaagaacgggaagtggacttgggccagatggcagcttggttaccatgcacagtggtaccataccaaagccagagtctcagagcaggggtccttagaagacataggcagtcggggatgtgagaagtgcttactgaagagtaaacaacagaatacaggcctcaccccagagatagcacgcacaaaataacacacaaaacctgcgggccttgagatagcacagacgtgacctgtgagcaacacatgattctgcccccacgagcagcctgggctcaccctacgtagtccaagatggccaggacaggccgaggggcccagcagaggcagctgagcagattgggcctggatcctgtgtcctgagcagagcacagggcaccttgagcctttttggtagcctctgtgacaaacgcagtgatgtagtcatttcccaggggaccactctatggctcctcgttctttgggagaaactttgaaacaacaacaaaattttatgagatttcctgagttgtgtgtgcacatgtgtggtgcacaaacaggacatgtgtgcatgtacatggctgtgttgtgtgcgtgtttgcacagtgtgaccacatgtgcactctgtgcatgtgtacatgtgtgttgtgcacgcatgcacacagatgtctacatgtgtgttttgtgcacatgtgcatgcacaaggcgcgcacgtgtgtgcatgttgacgtttgtgtgtgcacatatgattgtatatatgtgtgcctgtgtgtatatgtgtgcaagtgaactgaatgtgcgtgcatgcctgtgtggttgtatgtggatgtgtgtatgaatgacatgtgcacctgtgtgtgcatatgcccatgtgtgcatgtgtgtgcctgtggatgtgcacatgcccgtgtgtgcctgtggtgtgcacgtgtctgtggtgtgcacgtgcctgtgtgtgcatgtgcctttgtgtgtgtgccaccgtggtgcgtatgtgcctgtgtgtgcctgtgggtgtgcacgtgcctgtggtgtactcgtgcctgtggtgtgcatgtgcctgtggtgtgcatgtgcctgtggtgcgcatgtgcctgtgtgggtgtgcatgtgtgtgcctgtgggtgtgcacatgtctgtggtgtacacgtgcctgtggtgcgcatgtgcctgtgtcggtggattctgaaggcacgaaactgttcaccctggtgctgcttttcccagccttcctattgttgttgcttcaaccctgtggtgtgaagcctggtgacttgagaggagaattgcagtcacacttcttgcagcaatctggctgctcatttctgtgtccacccatctgtctaataacatctcctgggcatctgccatgtgctggggcaggggcccaaaggtgaaacaggcacatgctgtcagaacactcagccaggcttggcagtgagaccacatggtgactgtagctcaaggccagggcatttctgaggaaggagaggccatgtctggcctgaagaatgagaggaggtgccatttgagctgaacttaaaggaataggaaggattgagcactagagaaataggatggattgagctggggatgggaggtggggcagagggaagagggaggccaggtggcttctgtcaggctggagaggagagtgcattgcagaggggctgcaaggaaggctgagaccgtttgagaaaggcattgaaattggttgaacaagtcgctttgattttgcgttgtagacagtgggctcagtccttgcatcgaggagtgatggtgtcaggcccacactttccacctgggcttgatgggcagggacagctggggagggtggggggtctcacgtgtgtccagccccccgtggtggtcccttggctgccacaaccttagcaaacatctttggtgaggccgaggtggggcttcctgtttcttcccttttacagaaggaaattcaacatgatttggagctgattggaccgagggggctacactgtaggagagaggagcattggtggtgatccaaggatgccagcctacgatgagaggaggtccttagctggaccagggcttccaggggaggacacagagtgttcagggagacagggtcagtcaggaggcaaatgggtatgggcctctgaggttgctgtgatagagaagcagacctgccttgctgtggtggtgagaaagccaggcgtcagagggtttggctgacctgggaggcccagggagcatcgtcactcagggagtgagaggaagagctgaccgagagggaggggcagggcagtgttctgtgtggagcggctcctgcacacatcgggctccggaaatttcgctggggttgccacacctcccggggagctccaagccccagtaccccggcgaggtgccacctgtccagtgcacccctcacctccgccgccacgcctgcgcgaggtcggtcttcccatcacaggctgggcgggcgggagggtcgggctccatcctgcctttgttgccataacctgtgacatttcctttccagttgccggattcattataaggatatgtacagtttgttgcgttgtattgcgccacccgttggcttagggaagaactgccctcgtaggttggcctacaaggtttgcaacttcagagcctccatccaacatacttccctgcccgaaacgcacacagcacacactccctgcctggtttgggaatgacgacgcctcacctttctttcctcctgtcctgccccgcactccagcctcctggacgctttggttccctgtctaatctcccctgacccttctagctggctggggacaggcagccttttgagagtgcacagaaagctttctggttgggatcagctagctgggtgggagatgggggctaggtccctcctgcatcaaggaagcagccgacttgcccggtttttcctccgagctctcgagagtgagctgatggtcaggccaaaggccgagggccccagaggaagggagcacgggcacaggcctcccaggagcagccgtcctctgggttggaagagcccagtaacccagtggctctccagtccctgggggaggaaagggcagaggcagagtgggggccaaagacaagccgtcctgctttgcctctggagtgatgcgctcagctcagccaaggagcccctggtctggaaccgtgggaggagggcaaggccccagggcagatccatctgccaggaagaggctgtcccctttctctcctgggccacctgaatctggctgtgtctctaggaccccaagcccacaggcaggtgtcccagggttggaggtaggccccaggcctcgtggcctctctacttgggaagactctgtcatcggagcctttaggttcctagatcctgcaacctgcccctttgctgtcagagctggtgcaaggtggccccaggccatgtcctgccagctcctcccccctcccgcagtctcccatttcccacccagccctgagccccagcagcagccctgccctgggctggcctcgggcgtctctgggcttgaagcaggacttgccccaaggttaggttttggctgtacagcttcagggctccctgtttagttttctggtttgtttcagatcttctccacctttttagcctctagcctcacttggaagcaaatctgaccgtgaggaaagctgatgtctctgccgtggttctcagccactgcctcatctgccacctgaccctgcctgccagctcttctggcagggtggaccagctcttttctgattggcattcactgtgatttttaaaagagtaaaatcagaaattatcaacccaaagccgccccgatgcagcccttcctcacccctccatgttcattagctcagacgccacccccgcccactgccccgcgtggggctggagtgaggaggtgaggctcgggggtggggggccaggagggggtcaagccaagcagagaaatccctcattttatttattttgattttgggctttgaatccgacactttgattaactggctctgttttcttgtctgcttcctccctgcccctaccccgctcccctccccgctcccctcctgctgccgctcctcctgtggaccaccccactgtgccctggcctcgctgggacgggtttccagtgggcgcatcagttacaatgacatgtttgagatgctgaaacacatgtccccgcctctggg

SEQ ID NO: 36 - protein, 38 aa, Homo sapiens
YDPAACCRIHYKDMYSLLRCIAPPVGLGKNCPRRLAYK

SEQ ID NO: 37 - DNA, 6984 bp, Mus musculus
  CDS (1)..(6984)
atggtccgcttcggggacgagctaggcggccgctatgggggcaccggcggcggggagcgggctcggggcggcggggccggcggggcgggtggcccgggccaggggggtctgccgccgggccagcgggtcctgtacaagcagtccattgcgcagcgcgcacggactatggccctgtacaaccccatcccagtcaagcagaactgcttcaccgtcaaccgctcgctcttcgtcttcagcgaggacaacgtcgtccgcaaatacgctaagcgcatcaccgaatggccgcccttcgaatacatgatcctggccaccatcatcgccaactgtattgttctggccctggagcagcacctccctgatggggacaagactcccatgtctgagcgactagatgacacggagccttacttcatcgggatcttttgcttcgaggcgggcatcaagatcatagccctgggctttgttttccacaagggctcctaccttcggaacggctggaatgtcatggacttcgtggtggtcctcacggggattctcgccacagctggaactgactttgacctgcgcacactgagggctgtgcgtgtgcttaggcccctgaagctggtgtctggaattccaagcttgcaggtggtgcttaagtccatcatgaaggccatggtcccgctgctgcagattgggctgctgctcttctttgccatcctcatgtttgccatcatcggcctcgaattctatatgggcaaattccataaggcctgtttccccaacagcacagatacagagcctgtgggtgactttccctgtggcaaagatccccctgctcgtcagtgtgatggtgacaccgaatgccgggagtactggccaggacccaactttggcatcaccaattttgacaacatcctgtttgccatcttgacagtgttccagtgtatcaccatggagggctggactgacatcctctacaatacaaatgatgcggctggcaacacatggaactggttgtacttcatccccctcatcatcattggctccttcttcatgctcaacctggtgctgggtgtgctttccggagagtttgccaaggagcgggagcgagtcgagaaccgccgcgccttcctgaagctccgcaggcagcagcagattgagcgagagctgaatgggtacttggagtggatcttcaaggcagaggaagtcatgttggcagaggaggacaagaatgcagaagagaaatcccctttggatgtgttgaagagagctgccaccaagaagagccgaaatgacctcatccatgcagaagagggggaggaccggtttgtagacctctgtgcagttgggtctccatttgctcgtgccagcctcaagagtgggaagacggagagctcatcgtacttccggagaaaggagaagatgttccggttctttatccggcgtatggtgaaagcacagagcttctactgggtggtactgtgtgtggtggccctgaacacactgtgtgtggccatggtgcactataatcagcctcagcggcttaccactgcactgtactttgcagagtttgttttcctgggtctcttcctcacagagatgtccctgaagatgtatggcctagggcccagaagttacttcaggtcttccttcaactgctttgactttggggtgattgtggggagtatctttgaagtagtctgggctgccatcaagccaggaacctcctttggaatcagtgtgctgcgggctctgcgactgctgaggatattcaaagttaccaagtattggaactctctgaggaacctggtggtttccctcctcaattccatgaagtccatcatcagccttctcttcctgcttttcctcttcatcgtggtcttcgctctgttggggatgcagctgtttgggggacagttcaactttcaagatgagactccaaccaccaattttgataccttcccagctgccatcctcactgtctttcagatcctgacaggagaggattggaatgccgtaatgtatcatgggattgagtcgcaaggtggagtcagcaaaggcatgttttcttccttttacttcatcgtcctgacactgtttggaaactacaccctgctgaatgtttttctggccattgctgtggacaaccttgccaatgcccaggagttgaccaaggatgaagaggagatggaagaagcagccaatcagaaacttgctcttcagaaggccaaagaagtagctgaagtcagccccatgtctgctgccaatatctccatcgctgccaggcagcagaactcggccaaggcgcgctcagtatgggagcagcgggccagtcagctaaggctccagaatctgcgtgccagctgtgaggcattgtacagtgagatggaccctgaggagcgcctgcgttatgccagcacgcgccatgtgaggccagacatgaagacacacatggaccgacccctagtggtggagcctggtcgagatggcttgcggggacccgttgggagcaagtcaaagcctgaaggcacggaggccacagaaagcgcggacctacctcgccggcaccaccggcatcgtgatagggacaagacctcagccacagcacctgctggaggcgaacaggacaggacagaaagcaccgagaccggggcccgggaggaacgtgcgcgccctcgtcgaagtcacagcaaggagactccaggggctgacacgcaagtgcgctgtgagcgcagtaggcgtcaccaccggcgcggctccccggaggaggccactgaacgggagcctcggcgccaccgtgcccaccggcatgcacaggactcaagcaaggagggcacggcgccggtgcttgtacccaagggtgagcgacgagcaagacaccgaggcccacgcacgggtccacgtgaggcagagaacaacgaggagcccacacgcaggcaccgtgcaaggcataaggtgccacccacactgcagcccccagagagggaggctgcagagaaggagagcaacgcggtggaaggggataaggaaacccgaaatcaccagcccaaggaacctcactgtgacctggaggccattgcagttacaggtgtgggccctctgcacatgctgcccagcacctgtctccagaaagtggacgagcaaccagaggatgcagacaaccagcgtaatgtcacccggatgggcagtcagccctcagatcccagcaccactgtgcatgtcccagtgacactgacaggccctcctggggagacccctgtagttcccagtggtaacatgaacctggaaggccaagcagagggcaagaaggaggcagaggcggatgatgtgctgagaagaggccccaggcccatcgttccctacagctccatgttttgtctcagccccaccaacctgcttcgtcgcttctgccattacattgtgaccatgcggtactttgagatggtaattcttgtggtcattgccttgagcagcattgccctggctgcagaggatcctgtgcggacagattcattcaggaacaacgctttaaagtacatggattacatctttacaggagtcttcacctttgaaatggtcataaagatgatagacttgggcttgctgctgcaccctggtgcctacttccgggacctgtggaacattctggacttcatcgttgtcagtggagccctggtggcatttgcgttctcgagcttcatgggaggatccaaagggaaagacatcaataccatcaagtctctgagagtcctgcgtgtcctgaggcccctcaagaccatcaagcggctgcctaagctcaaggctgtctttgactgtgtggtgaactccctgaagaacgtcttgaacatcctgattgtctacatgctcttcatgttcatatttgccgtcattgccgtccagctcttcaaagggaagttcttttactgtactgatgaatccaaggagctggagagggactgccggggtcagtatttggattatgagaaggaagaagtagaagcccagccaaggcagtggaagaaatatgacttccactatgacaatgttctctgggccttgttgacgctgttcacagtgtccacgggagaggggtggcccatggtgctgaaacactctgtggatgccacctatgaggaacaggggcccagtcccgggttccggatggagctctccatcttctacgtggtctactttgtggtcttcccttttttctttgtcaacatctttgtggccttgatcattatcaccttccaggaacagggagataaggtgatgtctgaatgcagcttagaaaagaatgagagggcttgcattgattttgccatcagtgccaagcccctgacacggtacatgcctcaaaacaaacagtcgttccagtataagacatggacattcgtggtctctccaccctttgagtacttcatcatggctatgatagccctcaacacagtggtgctgatgatgaagttctatgatgcaccttatgagtacgagctgatgctgaaatgcctgaacattgtcttcacatccatgttctcgatggagtgcatactgaagatcatcgcctttggggtattgaactacttcagagatgcctggaacgtctttgactttgtcacggttttgggaagtattactgatattttagtaacagagatagcgaacaacttcatcaacctaagcttccttcgcctcttccgggcggcacggctgatcaagctgcttcgccagggctacaccatccgcatcctattgtggaccttcgtccagtcctttaaggcgctgccctacgtgtgcctcctcattgccatgctgttcttcatctacgccatcatcggaatgcaggtttttggaaacattgcccttgatgatgacaccagtatcaaccgacacaacaacttccggacatttctgcaagccttaatgctattgttcaggagtgccactggggaggcctggcatgagatcatgctgtcttgtctgggcaaccgggcctgtgacccacatgccaacgccagtgagtgcgggagcgactttgcctatttttattttgtctccttcatcttcctctgttcctttctgatgttgaacctctttgttgctgtaatcatggacaattttgagtacctcactcgggactcttccatcctagggcctcaccacttagacgaattcattcgagtctgggctgaatacgacccagctgcgtgtgggcgcatcagttacaatgacatgtttgagatgctgaaacacatgtccccacctctggggttggggaagaaatgcccggctcgagttgcatacaagcgcctggttcgcatgaacatgcccatatccaatgaggacatgacggtgcactttacgtccacactgatggccctcatccggacagcactggagatcaagcttgccccagcggggacgaagcagcaccagtgtgatgctgagctgagaaaagagatctcttctgtgtgggctaatctgccccagaagactctggacttactggtaccaccccacaaacctgacgagatgacagtggggaaggtctatgctgctctcatgatatttgacttctacaaacagaacaaaaccaccagagatcagactcaccaagctcccggaggcctgtcccagatgggtcccgtttccctgttccaccctctgaaggccaccctggaacagacacagcccgctgtgcttcgaggagctcgggttttccttcggcaaaagagtgcaacttccctcagcaatgggggtgccatacaaacccaggaaagtggcatcaaggagtcgctgtcctggggcacgcagaggacccaagatgcactttatgaggccagagcacctctagaacgtggccattctgcagagatccctgtggggcagtcaggaacactggctgtggatgtccagatgcagaacatgacactgagaggaccagatggggagccccagcctgggctggaaagccaaggcagagctgcctctatgccacgcctagcggcagaaacacagccggcccctaatgccagccccatgaagcgctccatctccacactggctccacgcccacatgggactcagctttgcagcacagttctggaccggcctcctcctagccaggcatcacatcaccaccaccaccgctgccaccggcgcagagacaagaagcaaaggtccctggaaaaggggcccagcctgtctgttgacccagaaggtgcaccaagcactgctgcaggacctggtctgccccatggagaaggatccaccgcctgccggcgggaccgtaaacaggagcgaggccggtcccaggagcggaggcagccctcatcttcctcttcagagaagcagcgcttctattcctgtgaccgctttgggagccgggagcccccgcaactgatgccctcactcagtagccaccccacatcgccaacagcggcgctagagccagcaccccacccacagggcagtggttccgttaatgggagccccttgatgtcaacatccggtgctagcaccccgggccgaggtgggcggaggcagctcccccagactcctctgaccccacgccccagcatcacctacaagaccgccaattcctcgcctgtccactttgctgagggtcagagcggcctcccagccttctcccctggccgtctcagccgcggcctttctgaacacaatgccctgctccagaaagagcccctgagccagcctctagctcctggctcccgaattggctctgacccttacctagggcagcgtctggacagtgaggcctccgcccacaccctgcctgaggatacactcacctttgaagaggcagtggccaccaactctggccgctcctccaggacttcctatgtgtcctccctcacttcccaatcccaccctctccgccgtgtacccaatggctatcactgcactttgggactcagcactggcgtccgggcgcggcacagctaccaccaccccgatcaggaccactggtgctag

SEQ ID NO: 38 - protein, 2327 aa, Mus musculus
MVRFGDELGGRYGGTGGGERARGGGAGGAGGPGQGGLPPGQRVLYKQSIAQRARTMALYNPIPVKQNCFTVNRSLFVFSEDNVVRKYAKRITEWPPFEYMILATIIANCIVLALEQHLPDGDKTPMSERLDDTEPYFIGIFCFEAGIKIIALGFVFHKGSYLRNGWNVMDFVVVLTGILATAGTDFDLRTLRAVRVLRPLKLVSGIPSLQVVLKSIMKAMVPLLQIGLLLFFAILMFAIIGLEFYMGKFHKACFPNSTDTEPVGDFPCGKDPPARQCDGDTECREYWPGPNFGITNFDNILFAILTVFQCITMEGWTDILYNTNDAAGNTWNWLYFIPLIIIGSFFMLNLVLGVLSGEFAKERERVENRRAFLKLRRQQQIERELNGYLEWIFKAEEVMLAEEDKNAEEKSPLDVLKRAATKKSRNDLIHAEEGEDRFVDLCAVGSPFARASLKSGKTESSSYFRRKEKMFRFFIRRMVKAQSFYWVVLCVVALNTLCVAMVHYNQPQRLTTALYFAEFVFLGLFLTEMSLKMYGLGPRSYFRSSFNCFDFGVIVGSIFEVVWAAIKPGTSFGISVLRALRLLRIFKVTKYWNSLRNLVVSLLNSMKSIISLLFLLFLFIVVFALLGMQLFGGQFNFQDETPTTNFDTFPAAILTVFQILTGEDWNAVMYHGIESQGGVSKGMFSSFYFIVLTLFGNYTLLNVFLAIAVDNLANAQELTKDEEEMEEAANQKLALQKAKEVAEVSPMSAANISIAARQQNSAKARSVWEQRASQLRLQNLRASCEALYSEMDPEERLRYASTRHVRPDMKTHMDRPLVVEPGRDGLRGPVGSKSKPEGTEATESADLPRRHHRHRDRDKTSATAPAGGEQDRTESTETGAREERARPRRSHSKETPGADTQVRCERSRRHHRRGSPEEATEREPRRHRAHRHAQDSSKEGTAPVLVPKGERRARHRGPRTGPREAENNEEPTRRHRARHKVPPTLQPPEREAAEKESNAVEGDKETRNHQPKEPHCDLEAIAVTGVGPLHMLPSTCLQKVDEQPEDADNQRNVTRMGSQPSDPSTTVHVPVTLTGPPGETPVVPSGNMNLEGQAEGKKEAEADDVLRRGPRPIVPYSSMFCLSPTNLLRRFCHYIVTMRYFEMVILVVIALSSIALAAEDPVRTDSFRNNALKYMDYIFTGVFTFEMVIKMIDLGLLLHPGAYFRDLWNILDFIVVSGALVAFAFSSFMGGSKGKDINTIKSLRVLRVLRPLKTIKRLPKLKAVFDCVVNSLKNVLNILIVYMLFMFIFAVIAVQLFKGKFFYCTDESKELERDCRGQYLDYEKEEVEAQPRQWKKYDFHYDNVLWALLTLFTVSTGEGWPMVLKHSVDATYEEQGPSPGFRMELSIFYVVYFVVFPFFFVNIFVALIIITFQEQGDKVMSECSLEKNERACIDFAISAKPLTRYMPQNKQSFQYKTWTFVVSPPFEYFIMAMIALNTVVLMMKFYDAPYEYELMLKCLNIVFTSMFSMECILKIIAFGVLNYFRDAWNVFDFVTVLGSITDILVTEIANNFINLSFLRLFRAARLIKLLRQGYTIRILLWTFVQSFKALPYVCLLIAMLFFIYAIIGMQVFGNIALDDDTSINRHNNFRTFLQALMLLFRSATGEAWHEIMLSCLGNRACDPHANASECGSDFAYFYFVSFIFLCSFLMLNLFVAVIMDNFEYLTRDSSILGPHHLDEFIRVWAEYDPAACGRISYNDMFEMLKHMSPPLGLGKKCPARVAYKRLVRMNMPISNEDMTVHFTSTLMALIRTALEIKLAPAGTKQHQCDAELRKEISSVWANLPQKTLDLLVPPHKPDEMTVGKVYAALMIFDFYKQNKTTRDQTHQAPGGLSQMGPVSLFHPLKATLEQTQPAVLRGARVFLRQKSATSLSNGGAIQTQESGIKESLSWGTQRTQDALYEARAPLERGHSAEIPVGQSGTLAVDVQMQNMTLRGPDGEPQPGLESQGRAASMPRLAAETQPAPNASPMKRSISTLAPRPHGTQLCSTVLDRPPPSQASHHHHHRCHRRRDKKQRSLEKGPSLSVDPEGAPSTAAGPGLPHGEGSTACRRDRKQERGRSQERRQPSSSSSEKQRFYSCDRFGSREPPQLMPSLSSHPTSPTAALEPAPHPQGSGSVNGSPLMSTSGASTPGRGGRRQLPQTPLTPRPSITYKTANSSPVHFAEGQSGLPAFSPGRLSRGLSEHNALLQKEPLSQPLAPGSRIGSDPYLGQRLDSEASAHTLPEDTLTFEEAVATNSGRSSRTSYVSSLTSQSHPLRRVPNGYHCTLGLSTGVRARHSYHHPDQDHWC

SEQ ID NO: 39 - DNA, 7185 bp, Mus musculus
  CDS (121)..(6987)
ggaattcggctcgaggggcgaggtccaggcagctcgctgcggctaggctaggagcccttggcgcgccgcgccctcggtgccgggccgcggagcccgggatgctcgcggcgcccgggagtcatggtccgcttcggggacgagctaggcggccgctatgggggcaccggcggcggggagcgggctcggggcggcggggccggcggggcgggtggcccgggccaggggggtctgccgccgggccagcgggtcctgtacaagcagtccattgcgcagcgcgcacggactatggccctgtacaaccccatcccagtcaagcagaactgcttcaccgtcaaccgctcgctcttcgtcttcagcgaggacaacgtcgtccgcaaatacgctaagcgcatcaccgaatggccgcccttcgaatacatgatcctggccaccatcatcgccaactgcattgttctggccctggagcagcacctccctgatggggacaagactcccatgtctgagcgactagatgacacggagccttacttcatcgggatcttttgctttgaggcgggcatcaagatcatagccctgggctttgttttccacaagggctcctaccttcggaacggctggaatgtcatggacttcgtggtggtactcacggggattctcgccacagctggaactgactttgacctgcgcacactgagggctgtgcgtgtgcttaggcccctgaagctggtgtctggaattccaagcttgcaggtggtgcttaagtccatcatgaaggccatggtcccgctgctgcagattgggctgctgctcttctttgccatcctcatgtttggcatcatcggcctcgagttctatatgggcaaattccataaggcctgtttccccaacagcacagatacagagcctgtgggtgactttccctgtggcaaagatccccctgctcgtcagtgtgatggtgacaccgaatgccgggagtactggccaggacccaactttggtatcaccaattttgacaacatcctgtttgccatcttgacagtgttccagtgtatcaccatggagggctggactgacatcctctacaatacaaatgatgcggctggcaacacgtggaactggttgtacttcatccccctcatcatcattggctccttcttcatgctcaacctggtgctgggtgtgctttctggagagtttgccaaggagcgggagcgagtcgagaaccgccgagccttcctgaagctccgcaggcagcagcagattgagcgagagctgaatgggtacttggagtggatcttcaaggcagaggaagtcatgttggcagaggaggacaagaatgcagaagagaaatcccctttggatgtgttgaagagagctgccaccaagaagagccgaaatgacctcatccatgcagaagagggggaggaccggtttgtagacctctgtgcagttgggtctccatttgctcgtgccagcctcaagagtgggaagacggagagctcatcgtacttccggagaaaggagaagatgttccggttctttatccggcgtatggtgaaagcacagagcttctactgggtggtactgtgtgtggtggccctgaacacactgtgtgtggccatggtgcactataatcagcctcagcggcttaccactgcactgtactttgcagagtttgttttcctgggtctcttcctcacagagatgtccctgaagatgtatggcctagggcccagaagttacttcaggtcttccttcaactgctttgactttggggtgattgtggggagtatctttgaagtagtctgggctgccatcaagccaggaacctcctttggaatcagtgtgctgcgggctctgcgactgctgaggatattcaaagttaccaagtattggaactctctgaggaacctggtggtttccctcctcaattccatgaagtccatcatcagccttctcttcctgcttttcctcttcatcgtggtcttcgctctgttggggatgcagctgttcggaggacagttcaactttcaagatgagactccaaccaccatttttgataccttcccagctgccatcctcactgtctttcagatcctgacaggagaggattggaatgccgtaatgtatcatgggattgagtcacaaggtggagtcagcaaaggcatgttttcttccttttacttcatcgtcctgacactgtttggaaactacaccctgctgaatgtttttctggccattgctgtggacaaccttgccaatgcccaggagttgaccaaggatgaagaggagatggaagaagcagccaatcagaaacttgctcttcagaaggccaaagaagtagctgaagtcagccccatgtctgctgccaatatctccatcgctgcgcaggagaactcggccaaggcgcgctcagtatgggagcagcgggccagtcagctaaggctccagaatctgcgtgccagctgtgaggcattgtacagtgagatggaccctgaggagcgcctgcgttatgccagcacgcgccatgtgaggccagacatgaagacacacatggaccgacccctagtggtggagcctggtcgagatggcttgcggggacccgttgggagcaagtcaaagcctgaaggcacggaggccacagaaagcgcggacctacctcgccggcaccaccggcatcgtgatagggacaagacctcagccacagcacctgctggaggcgaacaggacaggacagaaagcaccgagaccgggccccgggaggaacgtgcgcgccctcgtcgaagtcacagcaaggagactccaggggctgacacgcaagtgcgctgtgagcgcagtaggcgtcaccaccggcgcggctccccggaggaggccactgaacgggagcctcggcgccaccgtgcccaccggcatgcacaggactcaagcaaggagggcacggcgccggtgcttgtacccaagggtgagcgacgagcaagacaccgaggcccacgcacgggtccacgtgaggcagagaacaacgaggagcccacacgcaggcaccgtgcaaggcataaggtgccacccacactgcagcccccagagagggaggctgcagagaaggagagcaacgcggtggaaggggataaggaaacccgaaatcaccagcccaaggaacctcactgtgacctggaggccattgcagttacaggtgtgggccctctgcacatgctgcccagcacctgtctccagaaagtggacgagcaaccagaggatgcagacaaccagcgtaatgtcacccggatgggcagtcagccctcagatcccagcaccactgtgcatgtcccagtgacactgacaggccctcctggggagacccctgtagttcccagtggtaacatgaacctggaaggccaagcagagggcaagaaggaggcagaggcggatgatgtgctgagaagaggccccaggcccatcgttccctacagctccatgttttgtctcagccccaccaacctgtttcgtcgcttctgccattacattgtgaccatgcggtacttggagatggtaattcttgtggtcattgccttgagcagcattgccctggctgcagaggatcctgtgcggacagattcattcaggaacaacgctttagagtacatggattacatctttacaggagtcttcacctgtgaaatggtcataaagatgatagacttgggcttgctgctgcaccctggtgcctacttccgggacctgtggaacattctggacttcatcgttgtcagtggagccctggtggcatttgcgttctcaggatccaaagggaaagacatcaataccatcaagtctctgagagtcctgcgtgtcctgaggcccctcaagaccatcaagcggctgcctaaactcaaggctgtctttgactgtgtggtgaactccctgaagaacgtcttgaacatcctgattgtctacatgctcttcatgttcatatttgccgtcattgccgtccagctcttcaaagggaagttcttttactgtactgatgaatccaaggagctggagagggactgccggggtcagtatttggattatgagaaggaagaagtagaagcccagccaaggcagtggaagaaatatgacttccactatgacaatgttctctgggccttgttgacgctgttcacagtgtccacgggagaggggtggcccatggtgctgaaacactctgtggatgccacctatgaggaacaggggcccagtcccggcttccggatggagctctccatcctctacgtggtctactttgtggtcttcccttttttctttgtcaacatctttgtggccttgatcattatcaccttccaggaacagggagataaggtgatgtctgaatgcagcttagaaaagaatgagagggcttgcattgattttgccatcagtgccaagcccctgacacggtacatgcctcaaaacaaacagtcgttccagtataagacatggacattcgtggtctctccaccctttgagtacttcatcatggctatgatagccctcaacacagtggtgctgatgatgaagttctatgatgcaccttatgagtacgagctgatgctgaaatgcctgaacattgtcttcacatccatgttctcgatggagtgcatactgaagatcatcgcctttggggtattgaactacttcagagatgcctggaatgtctttgactttgtcacggttttgggaagtattactgatattttagtaacagagattgcggaaacgaacaacttcatcaacctaagcttccttcgcctcttccgggcggcacggctgatcaagctgcttcgccagggctacaccatccgcatcctattgtggaccttcgtccagtcctttaaggcgctgccctacgtgtgcctcctcattgccatgctgttcttcatctacgccatcatcggaatgcaggtttttggaaacagtgcccttgatgatgacaccagtatcaaccgacacaacaacttccggacatttctgcaagccataatgctattgttcaggagtgccactggggaggcctggcatgagatcatgctgtcatgtctggacaaccgggcctgtgacccacatgccaacgccagtgagtgcgggagcgactttgcctatttttattttgtctccttcatcttcctctgttcctttctgatgttgaacctctttgttgctgtaatcatggacaattttgagtacctcactcgggactcttccatcctagggcctcaccacttagacgaattcattcgagtctgggctgaatacgacccagctgcgtgtgggcgcatcagttacaatgacatgtttgagatgctgaaacacatgtccccacctctggggttggggaagaaatgcccggctcgagttgcatacaagcgcctggttcgcatgaacatgcccatatccaatgaggacatgacggtgcactttacgtccacactgatggccctcatccggacagcactggagatcaagcttgccccagctgacgagatgacagtggggaaggtctatgctgctctcatgatatttgacttctacaaacagaacaaaaccaccagagatcagactcaccaagctcccggaggcctgtcccagatgggtcccgtttccctgttccaccctctgaaggccaccctggaacagacacagcccgctgtgcttcgaggagctcgggttttccttcggcaaaagagtgcaacttccctcagcaatgggggtgccatacaaacccaggaaagtggatcaaggagtcgctgtcctgggggacgcagagggacccaagatgcactttatgagggcagagcacctctagaacgtgaccattctaaagagatccctgtggggcagtcaggaacactgctggtggatgtccagatgcagaacatgacactgagaggaccagatggggatccccagcctggcctggaaagccaaggcagagctgcctctatgctacgcctagcggcagaaacacagccggcccctaatgccagccccatgaagcgctccatctccacactggctccacgcccagatgggactcagctttgcagcacagttctggaccggcctcctcctagccaggcatcacatcaccaccaccaccgctgccaccggcgcagagacaagaagcaaaggtccctggaaaaggggcccagcctgtctgttgacccagaaggtgcaccaagcactgctgctgcaggacctggtctgccccatggagaaggatccaccgcctgccggcgggaccgtaaacaggagcgaggccggtcccaggagcggaggcagccctcatcttcctcttcagagaagcagcgcttctattcctgtgaccgcttgggagccgggagcccccaactgatgccctcactcagtagccaccccacatcgccggcggcggcgctagagccagcaccccacccacagggcagtggttccgttaatgggagccccttgatgtcaacatccggtgctattactcccgggcgaggtgggcggaggcagctcccccagactcctctgaccccacgccccagcatcacctacaagaccgccaattcctcgcctgtccactttgctgagggtcagagcggcctcccagccttctcccctggccgtctcagccgcggcctttctgaacacaatgccctgctccagaaagagcccctgagccagcctctagctcctggctcccgaattggctctgacccttacctagggcagcgtctggacagtgaggcctccgcccacaccctgcctgaggatacactcacctttgaagaggcagtggccaccaactctggccgctcctccaggacttcctatgtgtcctccctcacttcccaatcccaccctctccgccgtgtacccaatggctatcactgcactttgggactcaacactggcgtcggggcacgagcaagctaccaccaccccgatcaggaccactggtgctagcttcaccacgaccacccatgtaccagctccatgggtgagggttccagttgatgagttttatcatcccactctggactgtggggtcacaaccctgggaggagggccctcacatctcggcctctgtggtggaggctcctgcttccctccctccctccctttttacactggatagactaataaagccctttcttagagggg

SEQ ID NO: 40 - protein, 2288 aa, Mus musculus
MVRFGDELGGRYGGTGGGERARGGGAGGAGGPGQGGLPPGQRVLYKQSIAQRARTMALYNPIPVKQNCFTVNRSLFVFSEDNVVRKYAKRITEWPPFEYMILATIIANCIVLALEQHLPDGDKTPMSERLDDTEPYFIGIFCFEAGIKIIALGFVFHKGSYLRNGWNVMDFVVVLTGILATAGTDFDLRTLRAVRVLRPLKLVSGIPSLQVVLKSIMKAMVPLLQIGLLLFFAILMFGIIGLEFYMGKFHKACFPNSTDTEPVGDFPCGKDPPARQCDGDTECREYWPGPNFGITNFDNILFAILTVFQCITMEGWTDILYNTNDAAGNTWNWLYFIPLIIIGSFFMLNLVLGVLSGEFAKERERVENRRAFLKLRRQQQIERELNGYLEWIFKAEEVMLAEEDKNAEEKSPLDVLKRAATKKSRNDLIHAEEGEDRFVDLCAVGSPFARASLKSGKTESSSYFRRKEKMFRFFIRRMVKAQSFYWVVLCVVALNTLCVAMVHYNQPQRLTTALYFAEFVFLGLFLTEMSLKMYGLGPRSYFRSSFNCFDFGVIVGSIFEVVWAAIKPGTSFGISVLRALRLLRIFKVTKYWNSLRNLVVSLLNSMKSIISLLFLLFLFIVVFALLGMQLFGGQFNFQDETPTTIFDTFPAAILTVFQILTGEDWNAVMYHGIESQGGVSKGMFSSFYFIVLTLFGNYTLLNVFLAIAVDNLANAQELTKDEEEMEEAANQKLALQKAKEVAEVSPMSAANISIAAQENSAKARSVWEQRASQLRLQNLRASCEALYSEMDPEERLRYASTRHVRPDMKTHMDRPLVVEPGRDGLRGPVGSKSKPEGTEATESADLPRRHHRHRDRDKTSATAPAGGEQDRTESTETGPREERARPRRSHSKETPGADTQVRCERSRRHHRRGSPEEATEREPRRHRAHRHAQDSSKEGTAPVLVPKGERRARHRGPRTGPREAENNEEPTRRHRARHKVPPTLQPPEREAAEKESNAVEGDKETRNHQPKEPHCDLEAIAVTGVGPLHMLPSTCLQKVDEQPEDADNQRNVTRMGSQPSDPSTTVHVPVTLTGPPGETPVVPSGNMNLEGQAEGKKEAEADDVLRRGPRPIVPYSSMFCLSPTNLFRRFCHYIVTMRYLEMVILVVIALSSIALAAEDPVRTDSFRNNALEYMDYIFTGVFTCEMVIKMIDLGLLLHPGAYFRDLWNILDFIVVSGALVAFAFSGSKGKDINTIKSLRVLRVLRPLKTIKRLPKLKAVFDCVVNSLKNVLNILIVYMLFMFIFAVIAVQLFKGKFFYCTDESKELERDCRGQYLDYEKEEVEAQPRQWKKYDFHYDNVLWALLTLFTVSTGEGWPMVLKHSVDATYEEQGPSPGFRMELSILYVVYFVVFPFFFVNIFVALIIITFQEQGDKVMSECSLEKNERACIDFAISAKPLTRYMPQNKQSFQYKTWTFVVSPPFEYFIMAMIALNTVVLMMKFYDAPYEYELMLKCLNIVFTSMFSMECILKIIAFGVLNYFRDAWNVFDFVTVLGSITDILVTEIAETNNFINLSFLRLFRAARLIKLLRQGYTIRILLWTFVQSFKALPYVCLLIAMLFFIYAIIGMQVFGNSALDDDTSINRHNNFRTFLQAIMLLFRSATGEAWHEIMLSCLDNRACDPHANASECGSDFAYFYFVSFIFLCSFLMLNLFVAVIMDNFEYLTRDSSILGPHHLDEFIRVWAEYDPAACGRISYNDMFEMLKHMSPPLGLGKKCPARVAYKRLVRMNMPISNEDMTVHFTSTLMALIRTALEIKLAPADEMTVGKVYAALMIFDFYKQNKTTRDQTHQAPGGLSQMGPVSLFHPLKATLEQTQPAVLRGARVFLRQKSATSLSNGGAIQTQESGSRSRCPGGRRGTQDALYEGRAPLERDHSKEIPVGQSGTLLVDVQMQNMTLRGPDGDPQPGLESQGRAASMLRLAAETQPAPNASPMKRSISTLAPRPDGTQLCSTVLDRPPPSQASHHHHHRCHRRRDKKQRSLEKGPSLSVDPEGAPSTAAAGPGLPHGEGSTACRRDRKQERGRSQERRQPSSSSSEKQRFYSCDRLGAGSPQLMPSLSSHPTSPAAALEPAPHPQGSGSVNGSPLMSTSGAITPGRGGRRQLPQTPLTPRPSITYKTANSSPVHFAEGQSGLPAFSPGRLSRGLSEHNALLQKEPLSQPLAPGSRIGSDPYLGQRLDSEASAHTLPEDTLTFEEAVATNSGRSSRTSYVSSLTSQSHPLRRVPNGYHCTLGLNTGVGARASYHHPDQDHWC

SEQ ID NO: 41 - DNA, 7713 bp, Oryctolagus cuniculus
  CDS (121)..(7140)
ggccgggccgggcggggtccggtgcggtccggcagctccgcggcggcttcgtctcgatcgcctggcgcgccctccctgccggagccgccgggccggggatgcgcgcggcgcccggtggccatggtccgcttcggggacgagctgggcggccgctatgggggcgcgggcggcgcggagcgggcccggggcggcggggccggcggggctggcggcccgggccccggggggctgccgccgggccagcgggtcctctacaaacagtcgatcgcgcagcgcgcgcggaccatggcgctgtacaaccccatcccggtcaagcagaactgcttcaccgtcaaccgctcgctcttcgtcttcagcgaggacaacgtcgtccgcaaatacgccaagcgcatcaccgagtggcctccgttcgagtacatgatcctggccaccatcatcgccaactgcatcgtgctggccctggagcagcacctccctgacggggacaagacgcctatgtccgagcgcctggatgacaccgagccctacttcattgggatcttctgcttcgaggcggggatcaagatcctggccctgggctttgttctgcacaagggctcctatctgcggaacggctggaacgtcatggacttcgtggtagtcctcacggggattcttgccacagccggaaccgacttcgacctgcgaacgctgagggctgtgcgtgtgctaaggcccctgaagctggtgtctgggattccgagcttgcaggtggtgctcaagtccatcatgaaggccatggtcccgctgctgcagatcgggctgctgctcttcttcgccatcctcatgtttgccatcatcggcctcgagttctacatgggcaagttccacaaggcctgcttccccaacagcacagacccagatcccgtgggtgacttcccctgtggcaaggaggccccggcccggctctgtgagggagacacggagtgccgggagtactgggccggacccaactttggtatcaccaactttgacaacatcctgtttgccatcttgaccgtgttccagtgcatcaccatggagggctggactgacatcctctacaatacaaacgatgcagccggcaacacgtggaactggctctacttcatccctctcatcatcatcggctccttcttcatgctcaacctggtgctgggtgtgctctcaggagagtttgctaaggagcgcgagagagtggagaaccgccgcgccttcctgaagctccggcggcagcagcagattgagcgggagctcaacgggtacctggagtggatcttcaaggctgaggaagtcatgctggctgaggaggacaggaacgcagaggagaagtcccctctggatgcagtgttgaagagagcagctgccaagaagagcagaagcgacctgatccaggccgaggagggggagggccggctcacaggcctctgtgctcccggttccccgttcgcccgtgccagcctgaaaagcgggaagacggagagctcgtcgtacttccggaggaaggagaagatgttccgcttcttcatccggcgcatggtgaaggcacagagcttctactggacggtgctgtgtgtggtggcactgaacacactgtgcgtggccatggtgcactacaaccagccgcagcggctcaccacggcattgtactttgcagagtttgttttcctgggtctcttcctcacagagatgtccctgaagatgtatggcctggggcccaggagctacttccggtcctccttcaactgttttgactttggggtcatcgtgggcagtatctttgaagtggtctgggctgctgtcaagccagggacctccttcgggatcagtgttctgcgggctctgcgcttactgagaatcttcaaggtcaccaaatactggaactccctacggaacctggtggtgtctctgctcaactccatgaagtccatcatcagcctcctcttcctgctcttcctgttcattgttgtctttgctctgctggggatgcagctgtttggaggccagttcaactttaaagacgagaccccaacgaccaactttgacaccttccctgctgctatcctcaccgtcttccagatcctgaccggagaggactggaatgcggtcatgtaccacgggatcgagtcgcaaggtggcgtcagcagaggaatgttctcctccttctacttcattgtcctcactctgtttgggaactacaccctgctgaatgtcttcctggccattgctgtggacaaccttgccaatgctcaggagctgaccaaggatgaagaggagatggaagaagcagccaatcagaaactcgccttgcagaaggccaaggaagtggctgaagtcagccccatgtctgctgcgaacatctccattgccgccaggcagcagaactcggccaaagcgcgctcggtgtgggagcagcgagccagccagctgcggctgcagaacctgagggccagctgcgaggcgctgtacagcgagatggaccccgaggagaggctgcgctacgccaccgcgcgccacctgcggcccgacgtgaagacgcacctggaccggccgctggtggttgagcccggccgcgacgctccgcggggccccccgggggggaagtcccggccggatggctcggaggcccccgagggtgcggatcccccgcgcaggcatcaccggcaccgagacaaggacaaggctcccgccacggtcccctcggcgggggaacaggacagagccgaagctctgagggcagagggcggggagctggggccccgcgaggagcgggggcggccacgccgcagccgcagcaaggaggccccgggcgccccggaggtgcgcagcgatcgcggccgtggcccgtgtcctgaaggtggtcggcgccaccaccggcgcgggtcaccggaggaggcagctgagagggagccccgccgccaccgcgcgcaccggcacgggcccgacccgggcaaggagggcccagcgtccgggaccaggggcgagcggcgcgcacgacaccgcacgggcccccgggcgtgcccccgggaggctgagagcagcgaggagcctgcgcggcggcaccgggctcggcacaaggcgccgcccacccaggagaccgcggagaaggacaaggaggctgccgagaaggggggcgaggccacggaggccgagaaggacaaggaggctcggaaccaccagcccaaggagctgccctgtgacctggaggccatcggcatgctgggagtgggcgctgtgcatacgctgcccagcacctgtctgcagaaggtggaggagcagccagaggatgcggacaaccagcggaacgtcacccgcatgggtagtcagcccccggacaccagcaccactgtgcacattcctgtgacgctgaccggcccccccggggagaccacggtagtgcccagtggaaacgtggacctggaaagtcaagcagaggggaagaaggaggtggaaaccagcgatgtgatgaggagcggccctcggcccattgtcccgtacagttccatgttctgtttaagcccaaccaacctgctgcgccgctgctgccactacattgtgaccatgcgatacttcgagatggtcatcctcgtggtcatcgccctgagcagcatcgccctggctgccgaggaccccgtgagaacagactctcccaggaataatgctctgaagtacatggactacatcttcacaggcgtcttcaccttcgagatggtgataaagatgatcgatttgggactgctgctacaccctggagcctatttccgggatctgtggaacattctggacttcattgtggtcagtggggccctggtggcatttgctttctcaggatccaaagggaaagacatcagcaccatcaaatccctgagagtcctgcgtgtgctgcggccactcaagaccatcaagcggctgcccaagctcaaggccgtgtttgactgtgtggtgaactcactgaaaaacgtcctcaacatcctgatcgtctacatgctcttcatgttcatctttgccgtcattgccgtgcagctcttcaaagggaagttcttctactgcacggacgaatccaaggagctggagcgtgactgcaggggtcagtatttggattatgagaaggaggaagtggaagctcaaccgaggcagtggaagaaatatgacttccactatgacaacgtgctctgggctctgctaacgctcttcacggtgtccacaggggaagggtggcccatggtgctgaaacactctgtggatgccacctacgaggagcagggcccgagccccgggtaccgcatggagctctccatcttctacgtggtctactttgtggtctttcccttcttctttgtcaacatcttcgtggccttgatcatcatcaccttccaggagcagggggacaaggtgatgtccgagtgcagcctggagaagaacgagagggcttgcatcgactttgccatcagcgccagacccctgacgcggtacatgccccagaacaagcagtcgttccagtataagacgtggacgttcgtggtgtccccaccttttgagtacttcatcatggctatgatcgccctcaacactgtggtgctgatgatgaagttctacgatgcgccgtacgagtatgagctaatgctgaaatgcctgaacatcgtgttcacgtccatgttctccatggagtgtgtgctgaagatcatcgccttcggggtgctgaactatttcagagatgcctggaatgtctttgactttgtcacggtgttgggaagtattactgatattttggtaacagagattgcgaacaacttcatcaacctcagcttcctgcgcctgttccgggccgcgcggctcatcaagctgctgcgccagggctacaccatccgcatcctgctttggaccttcgtgcagtccttcaaggcgctgccctacgtgtgtctcctcatcgccatgctcttcttcatctacgccatcattggcatgcaggtttttgggaacatcgccctggacgatgacaccagcatcaacaggcacaataacttccggacgtttctgcaggccttgatgttgctgttcaggagcgccacgggggaggcctggcacgagataatgctgtcgtgcctcagcagccgggcctgcgacgagcactccaatgccagcgagtgcggcagcgactttgcctacttttacttcgtctccttcatcttcctctgctcctttctgatgttgaacctctttgtggctgtgatcatggacaattttgagtaccttacacgggactcttccatcttagggcctcaccacctggatgagttcatccgggtctgggctgaatacgacccggccgcgtgtgggcgcatcagttacagtgacatgtttgagatgctgaagcacatgtccccacccctggggctggggaagaagtgccctgctcgagtcgcgtacaagcgcctggtccgcatgaacatgcccatctccagtgaggacatgacggtgcacttcacatccacgctgatggctctcattcggacagcgctggacatcaagctggcaccagctgggaccaagcagcaccagtgcgacgcagagctgaggaaggagatttcctgtgtgtgggccaatctgccccagaagaccctggacttactggtaccaccccataagcctgacgagatgacggtgggcaaagtctatgcagctctgatgatcttcgacttctacaaacagaacaaaaccagcagagaccagactcagcaagcccccggaggcctgtcccagctgggccccgtgtccctgttccacccgctgaaggccacgctggagcagactcagccggcgctgcgtggagcccgggctttcctgcggcagaagagttctgcctccctgagcaacggcggggccgtacaaacccaggagagcggcatcaaggaatcggtttcttggggcactcagaggacccaggatgtgctttgcgaggccagggcacccctggagcggggccactcagctgagatcccagtcgggcagcctgggacactggctgtagatgtccagatgcagaacatgactctgagtggccctgacgccgagccgcagccagggctggagagccagggccgagccgcctccatgccccgcctggccgcagaaacacagcccgcgcctgacgccagccccatgaagcgctccatctccacgctggccccgcgcccccacacggcccggctcggcagcaccgcgctggaccgcccagcccccagccaggccccgcaccatcaccaccaccgctgccaccgccgcagggacaggaagcagcggtccctggagaagggtcccagcctgtctgctgacacagatggggcccccgacagcacggtggggccaggactgcccacgggggagggcccccccggctgcaggcgggagcgggagcgcaggcaggagcggggccggtcccaggagcggaggcagccctcgtcctcctcctcggagaagcatcgcttctactcctgtgaccgcttcgggggccgcgaacccccacagcccaagccctccctcagtagtcaccccacgtcgccgacggccgggcaggagccgggaccccacccgcaggggagtggttcggttcacgggagccccctgctgtccacatctggtgctagcaccccgggccgggggcggaggcagctcccccagacccccttgaccccccgccccagcgtcacctacaagacggccaactcctcgcctgtccacttcgccggggcaccgagcggcctccccgccttctcccccggccggctcagccggggcctctcagaacacaacgccctgctgcagagggacccgctcagccggcccctggcgcccggctcgcgcatcggctccgacccctacctggggcagcggctggacagcgaggcgcccgcccgtgccctgcccgaggacgcgcccgcctttgaggagaccgcggccagcaactcgggccgctcctcccggacctcgtacgtgtcctcactgacctcgcagcccccgccgctgcgccgcgtgcccaacggctaccactgcactctggggctgggcgggggcggccgggcgcggcgcggctgccaccaccccgaccgcgaccgccggtgctagccgctgcgcgcccgcccccggcggtgggcttcatcttccgcgctgggctgcggggacgcgggcaccgaggccctggacggggcctgggcggaggcccctgctcccctccccctcctccctgacagcagacggatcaataaagccccttccggcgggacgcggctgtctctgccccctgacgtgctgccttcccaggtctcccaccacaggctgatcctgtgtcagagaccccggtgtcagtgtgcaggggtggggggtgcggggcctgtgtcggacccccagggttaatggggggagcgtcagtgtgtgggagcacctgtgacccgtgttggtgggaggagttgcactgcctggaggcataggggagcttaggggtctcgagggagccgggggaggagctgaggttgctcctggtccctggtcgcggggcggcaggtggacgctggacacactcagatctctcagtccctccccgaccccatcagccccaacagggaccaaacctggaggacgtgccggctgctggtctaaacagaacaggaagaaaatgaggaaaccggaaaatcagagccg

SEQ ID NO: 42 - protein, 2339 aa, Oryctolagus cuniculus
MVRFGDELGGRYGGAGGAERARGGGAGGAGGPGPGGLPPGQRVLYKQSIAQRARTMALYNPIPVKQNCFTVNRSLFVFSEDNVVRKYAKRITEWPPFEYMILATIIANCIVLALEQHLPDGDKTPMSERLDDTEPYFIGIFCFEAGIKILALGFVLHKGSYLRNGWNVMDFVVVLTGILATAGTDFDLRTLRAVRVLRPLKLVSGIPSLQVVLKSIMKAMVPLLQIGLLLFFAILMFAIIGLEFYMGKFHKACFPNSTDPDPVGDFPCGKEAPARLCEGDTECREYWAGPNFGITNFDNILFAILTVFQCITMEGWTDILYNTNDAAGNTWNWLYFIPLIIIGSFFMLNLVLGVLSGEFAKERERVENRRAFLKLRRQQQIERELNGYLEWIFKAEEVMLAEEDRNAEEKSPLDAVLKRAAAKKSRSDLIQAEEGEGRLTGLCAPGSPFARASLKSGKTESSSYFRRKEKMFRFFIRRMVKAQSFYWTVLCVVALNTLCVAMVHYNQPQRLTTALYFAEFVFLGLFLTEMSLKMYGLGPRSYFRSSFNCFDFGVIVGSIFEVVWAAVKPGTSFGISVLRALRLLRIFKVTKYWNSLRNLVVSLLNSMKSIISLLFLLFLFIVVFALLGMQLFGGQFNFKDETPTTNFDTFPAAILTVFQILTGEDWNAVMYHGIESQGGVSRGMFSSFYFIVLTLFGNYTLLNVFLAIAVDNLANAQELTKDEEEMEEAANQKLALQKAKEVAEVSPMSAANISIAARQQNSAKARSVWEQRASQLRLQNLRASCEALYSEMDPEERLRYATARHLRPDVKTHLDRPLVVEPGRDAPRGPPGGKSRPDGSEAPEGADPPRRHHRHRDKDKAPATVPSAGEQDRAEALRAEGGELGPREERGRPRRSRSKEAPGAPEVRSDRGRGPCPEGGRRHHRRGSPEEAAEREPRRHRAHRHGPDPGKEGPASGTRGERRARHRTGPRACPREAESSEEPARRHRARHKAPPTQETAEKDKEAAEKGGEATEAEKDKEARNHQPKELPCDLEAIGMLGVGAVHTLPSTCLQKVEEQPEDADNQRNVTRMGSQPPDTSTTVHIPVTLTGPPGETTVVPSGNVDLESQAEGKKEVETSDVMRSGPRPIVPYSSMFCLSPTNLLRRCCHYIVTMRYFEMVILVVIALSSIALAAEDPVRTDSPRNNALKYMDYIFTGVFTFEMVIKMIDLGLLLHPGAYFRDLWNILDFIVVSGALVAFAFSGSKGKDISTIKSLRVLRVLRPLKTIKRLPKLKAVFDCVVNSLKNVLNILIVYMLFMFIFAVIAVQLFKGKFFYCTDESKELERDCRGQYLDYEKEEVEAQPRQWKKYDFHYDNVLWALLTLFTVSTGEGWPMVLKHSVDATYEEQGPSPGYRMELSIFYVVYFVVFPFFFVNIFVALIIITFQEQGDKVMSECSLEKNERACIDFAISARPLTRYMPQNKQSFQYKTWTFVVSPPFEYFIMAMIALNTVVLMMKFYDAPYEYELMLKCLNIVFTSMFSMECVLKIIAFGVLNYFRDAWNVFDFVTVLGSITDILVTEIANNFINLSFLRLFRAARLIKLLRQGYTIRILLWTFVQSFKALPYVCLLIAMLFFIYAIIGMQVFGNIALDDDTSINRHNNFRTFLQALMLLFRSATGEAWHEIMLSCLSSRACDEHSNASECGSDFAYFYFVSFIFLCSFLMLNLFVAVIMDNFEYLTRDSSILGPHHLDEFIRVWAEYDPAACGRISYSDMFEMLKHMSPPLGLGKKCPARVAYKRLVRMNMPISSEDMTVHFTSTLMALIRTALDIKLAPAGTKQHQCDAELRKEISCVWANLPQKTLDLLVPPHKPDEMTVGKVYAALMIFDFYKQNKTSRDQTQQAPGGLSQLGPVSLFHPLKATLEQTQPALRGARAFLRQKSSASLSNGGAVQTQESGIKESVSWGTQRTQDVLCEARAPLERGHSAEIPVGQPGTLAVDVQMQNMTLSGPDAEPQPGLESQGRAASMPRLAAETQPAPDASPMKRSISTLAPRPHTARLGSTALDRPAPSQAPHHHHHRCHRRRDRKQRSLEKGPSLSADTDGAPDSTVGPGLPTGEGPPGCRRERERRQERGRSQERRQPSSSSSEKHRFYSCDRFGGREPPQPKPSLSSHPTSPTAGQEPGPHPQGSGSVHGSPLLSTSGASTPGRGRRQLPQTPLTPRPSVTYKTANSSPVHFAGAPSGLPAFSPGRLSRGLSEHNALLQRDPLSRPLAPGSRIGSDPYLGQRLDSEAPARALPEDAPAFEETAASNSGRSSRTSYVSSLTSQPPPLRRVPNGYHCTLGLGGGGRARRGCHHPDRDRRC

SEQ ID NO: 43 - DNA, 7121 bp, Bos taurus
  CDS (126)..(7121)
ctgggcgaggtctggtgcgggtccggcggctccgcggctgctccgctagagcgcagggcgcacctcgcgccttccgatccccgcggggccgccgggccgggggatgcgctcagcgcccgggagccatggtccgcttcggggacgagctgggcggccgctatgggggccccggcggtggggagcgggcgcggggcggcggggccggcgttgcgggcggcccgggccccggggggctgcagccgggccagcgggtcctctacaagcagtcgatcgcgcagcgcgcgcggaccatggcgctgtacaaccccatcccggtcaagcagaactgcttcaccgtcaaccgctcgctcttcgtcttcagcgaggacaacgtcgtccgcaaatacgccaagcgcatcaccgagtggcctccgtttgagtacatgattctggccaccatcatcgccaactgtatcgtgctggctctggagcagcacctcccagacggagacaagacgcccatgtctgagcggctggacgacacggagccctacttcatcggcatcttctgcttcgaggctgggatcaagatcatcgcgctgggctttgtcttgcacaagggctcctacctgcggaatggctggaacgtcatggacttcgtggtggtcctcacggggatcctcgccacagctggcactgactttgatctgcgaacactgcgggctgtgcgtgtactgaggcccctgaagctggtgtctgggattccaagtttgcaggtggtgctcaagtccatcatgaaggccatggtcccgctcctgcagattgggctgcttctctttttcgccatcctcatgttcgccatcattggcctcgagttctacatgggcaaattccacaaggcttgtttccccaacagcacagacgcagatccggtgggtgacttcccgtgcggcagggaggccccggcgcggctgtgcgagggcgacactgagtgccgggagtactgggcgggccccaacttcggcatcaccaacttcgacaacatcctgttcgccatcctgacggtgttccagtgcatcaccatggagggctggaccgacatcctctacaacaccaacgacgcggctggcaacacctggaactggctctacttcctgcccctgattatcatcggctcgttcttcatgctcaacctggtgctgggcgtgctctcaggagagtttgccaaggagcgtgagcgggtggagaaccgccgggcctttctgaagctccgccggcagcagcagatcgagcgggagctcaatgggtacctggagtggatcttcaaggccgaggaggtcatgctggccgaggaggacaagaacgcagaggagaagtcccctctggatgtgttgaagagagcagccaccaagaagagccgaaacgacctgatccacgcggaggagggagaggaccgctttgcagacctctgtgctgtggggtcccccttcgcccgagccagcctcaagagtgggaagacggagagctcgtcatacttccgcaggaaggagaagatgttccggttcttcatccggcgcctggtgaaggcgcagagcttctactgggtggtgctgtgtgtggtggccctgaacacgctgtgcgtggccatggtgcactaccagcagccccagcggctcaccacggcgctgtactttgcagagtttgttttcctgggtctcttcctcacagagatgtccctgaagatgtatggcctggggcccaggagctacttccggtcctccttcaactgcttcgatttcggggtcattgtggggagcattttcgaagtggtctgggcggccatcaagccaggaacctccttcggcatcagtgtgctgcgggcgctgcggctgctgagaatcttcaaagtcaccaagtactggagctccctgcggaacctggtggtgtccctgctcaactccatgaagtccatcatcagcctgctcttcctgctcttcctcttcatcgtggtcttcgctctgctgggcatgcagctcttcgggggacagttcaacttccaggatgagactccgacaaccaactttgacaccttccctgccgccatcctcactgtcttccagatcctgaccggggaggactggaatgcggtgatgtaccatgggatcgaatcacaaggtggagtcagcaagggcatgttctcgtctgtgtacttcattgtcctgacgctgtttgggaactacaccctgctgaacgtcttcctggccatcgctgtggacaacctcgccaatgctcaggagctgaccaaggacgaagaggagatggaagaagcagccaatcagaagcttgctctgcaaaaggccaaagaagtggctgaagtcagccccatgtctgccgcgaacatctccattgctgccaggcagcagaactcggccaaggcgcgctcggtgtgggagcagcgggccagccagctgcggctgcagaacctgcgggccagctgtgaagcgctgtatagcgagatggaccccgaggagcgcctgcgcttcgccacttcccgccacctgcggcccgacatgaagacgcacctggaccggccgcttgtggtggagccaggccgggatggcgcgcgggggcctgcggggggcaaggcccggcccgagggcggagaggccggcgagggtgccgacccgccgcgcaggcaccaccggcaccgcgacagggacagggacaaggccgcagccccggcgggggagccagacagggcggacgccccgaaggcggagggcggggagccgggggcccgggaggagcgggcgcgaccgcgccgcagccgcagcaaggaggccgcaggcccccgcgaggccaggagcgagcgaggccgcgggctgggcccggacggcggccggcgccaccaccggcgcggctccccggaggaggccgcggagcgcgagccccggcgccaccgcgcgcaccggcacgcgccggagccgagcagggagggcgcgcccgggagcaagggcgagcggcgagcgcggcaccgcggcggctcccgggccgggccccgggaggccgagagcggcgaggagccctcccggcggcaccgagcgcgacacaaggcgccgcccgcacacgaggaggcggagaaggaggccgaggcggaggacagggacaaggagccccgaaaccaccagcccagggagtcgcactgtgacctggaggccagtgtggtcattggtgtgggccctgtgcatgccctgcccagcacttgtctagagaaggtggaagagcagccagaggatgcagacaatcagcggaatgtgactcgcatgggcagtcagccctcagatctgagcaccgctgtgcgtatcccagtgacgctgaccgggcctcccggggagaccacggttgttcccagtggtaacgtggacctggaaagccaagcagaaggaaagaaggaggtggaagctgatgacgtgatgaggagtggcccccgacccattgtcccgtacagctccatgttctgcttgagccccaccaacctgctccgccgcttctgccactacattgtgaccatgcagtacttcgagatggtcattctcgtggtcattgccctgagtagcattgccctggctgcggaggatcctgtgcagacagactcgcccaggaacaacgtgctgaagtacatggactacatcttcacaggtgtcttcacctttgagatggtgataaagatgatcgatttgggactgctgctgcaccccggtgcctacttccgggatctgtggaacattctggacttcattgtggtcagtggggccctggtggcgtttgccttctcaggctccaaagggaaagacatcagcaccatcaagtccctgagggtcctgcgtgtgctgaggccactcaagaccatcaagcggctgcccaagctcaaagctgtgtttgactgtgtggtgaactccctgaagaacgtcctcaacatcctgatcgtctacatgctcttcatgttcatatttgctgtcatcgcggtacagctcttcaaggggaagtttttctactgcaccgacgagtccaaggagctggagcgggactgcaggggccagtatttggactacgagaaggaggaggtggaagctcagccgcggcagtggaagaaatacgacttccactatgacaacgtgctgtgggccctgctaacgctgttcactgtgtccacgggcgaagggtggcccatggtgctgaaacactcggtggatgccacctacgaggagcagggccccagccccgggttccgcatggagctgtccatcttctatgtggtctacttcgtggtcttccccttcttctttgtgaacatctttgtggccttgatcatcatcaccttccaggagcagggggacaaagtcatgtctgagtgcagcttggagaagaacgagagggcttgtatcgacttcgccattagcgccaaacccctgacgcggtacatgccccagaacaagcagtcgttccagtacaagacgtggacgttcgtggtctcaccaccctttgagtatttcatcatggccatgatagccctcaacacagtggtgctgatgatgaagttctacgatgcgccctacgagtacgaactgatgttgaagtgtctgaacatcgtgttcacgtccatgttctccatggagtgtgtgctcaaggtcatcgcctttggagtgctgaactatttcagagacgcctggaatgtctttgactttgtcactgtgttgggaagtattactgatattttagtaacagagattgcgaacaacttcatcaacctcagcttcctccgcctcttccgtgctgcccgactcatcaagctgctccgccaaggctacaccatccgcattctgctctggaccttcgtccagtccttcaaggccctgccctatgtgtgcctgctcatcgccatgctgttcttcatctacgccatcatcggcatgcaggtgtttggaaacattgctctggatgacgacaccagcatcaaccgtcacaataactttcggacatttcttcaggccttgatgctgctcttcaggagtgccaccggggaggcctggcatgagatcatgctgtcctgcctcagcagtcgggcctgtgatgagctcgccaatgccacggagtgtgggagtgacttcgcctacttctactttgtctccttcatcttcctttgctcctttctgatgttgaacctctttgtggctgtgatcatggacaattttgagtaccttacgcgggactcttccatcctagggcctcaccacctggacgaattcatccgggtctgggccgagtacgacccggctgcgtgtgggcgcatcagttacaatgacatgtttgagatgctgaaacacatgtccccgcccctggggctggggaagaaatgccctgctcgtgttgcgtacaagcgcctggttcgtatgaacatgcccatctccaatgacgacatgaccgtccacttcacatccacgctgatggccctcatccggactgcattggagatcaagctggctccagctggcacgaagcagcatcagtgtgacgcagagctgaggaaggagatctcctccgtgtgggccaatctgccccagaagaccctggacttactggtgccaccccataaacccgacgagatgacggtggggaaggtgtacgcagctctgatgatattcgacttctacaaacagaacaaaaccaccagagaccagattcaccaagctcctggaggcctggcccagatgggccccgtgtccctgttccaccccctgaaggccacgctggagcagactcagccggctgtgctccgaggagcccgcgttttccttcggcagaagagctcggcctccctcagcaacggtggggcagtacaaacccaagagggcggcatcaaggagtctgtctcctggggcactcagaggacccaggaggtcccctgcgaggtgaggacacccctggagcgtggccactccacagagatcccggtgcagacaggaaaaccggctgtggacgtccagatgcagagcatggtcctgagaggccctgacggggagccccaacctgggctggagagccagggccgagcagcttccatgccccgcctggcagcagagactcagccacccccagatgccagccccatgaagcgctccatctccacactggcaccccagaggccccacgtggctcatctgtgcaccgcggccctggaccgcgctccagccagccaggccgcccccccccaccaccaccgctgccatcgccgcagggaccggaagcagaagtccctggagaaggggcccggcctgtctgcagacacagatggcgcgccctacagcaccgcggggccgggccccccgccgcccccaggggatggggcagcaggctgccggcgggagcgcaggcaggagcggggccggtcccaggagcggaggcagccctcctcctcttcctctgagaagcagcgcttctactcctgtgaccgctttgggggccgtgagcccccgcagcccaagccctctctcagcagccatcccacgtcgcccacggccgggcaggagccaggacccccgagacagggtagcggctccgtgaacgggagccccctgctgtcgacatctggtgctagcacccctggccgcggcgggcggaggcagctcccccagaccccgctgaccccccgtcccagcatcacctataagacggccaactcctcacctgtccactttgccggggctcagaccagcctccctgccttttcccccggccggcttagccgtggcctttcggaacacaacgccctgctccagagagaccccctcagccagccgctggcccccagctctcggattggctccgacccttacctggggcagcgtctggacagtgaggctgcggcccgcacccagcttgaggacacgctcaccttcgaggaggccgtggccaccaactcgggccgctcctcccggacttcctacgtgtcctccctgacctcgcagtcccaccccctccgccgggtgcccaacggctaccactgcactctggggctcagctcgggcgggggcggccgggggcggcacagctaccaccaccctgaccaagaccactggtgctag

SEQ ID NO: 44 - protein, 2331 aa, Bos taurus
MVRFGDELGGRYGGPGGGERARGGGAGVAGGPGPGGLQPGQRVLYKQSIAQRARTMALYNPIPVKQNCFTVNRSLFVFSEDNVVRKYAKRITEWPPFEYMILATIIANCIVLALEQHLPDGDKTPMSERLDDTEPYFIGIFCFEAGIKIIALGFVLHKGSYLRNGWNVMDFVVVLTGILATAGTDFDLRTLRAVRVLRPLKLVSGIPSLQVVLKSIMKAMVPLLQIGLLLFFAILMFAIIGLEFYMGKFHKACFPNSTDADPVGDFPCGREAPARLCEGDTECREYWAGPNFGITNFDNILFAILTVFQCITMEGWTDILYNTNDAAGNTWNWLYFLPLIIIGSFFMLNLVLGVLSGEFAKERERVENRRAFLKLRRQQQIERELNGYLEWIFKAEEVMLAEEDKNAEEKSPLDVLKRAATKKSRNDLIHAEEGEDRFADLCAVGSPFARASLKSGKTESSSYFRRKEKMFRFFIRRLVKAQSFYWVVLCVVALNTLCVAMVHYQQPQRLTTALYFAEFVFLGLFLTEMSLKMYGLGPRSYFRSSFNCFDFGVIVGSIFEVVWAAIKPGTSFGISVLRALRLLRIFKVTKYWSSLRNLVVSLLNSMKSIISLLFLLFLFIVVFALLGMQLFGGQFNFQDETPTTNFDTFPAAILTVFQILTGEDWNAVMYHGIESQGGVSKGMFSSVYFIVLTLFGNYTLLNVFLAIAVDNLANAQELTKDEEEMEEAANQKLALQKAKEVAEVSPMSAANISIAARQQNSAKARSVWEQRASQLRLQNLRASCEALYSEMDPEERLRFATSRHLRPDMKTHLDRPLVVEPGRDGARGPAGGKARPEGGEAGEGADPPRRHHRHRDRDRDKAAAPAGEPDRADAPKAEGGEPGAREERARPRRSRSKEAAGPREARSERGRGLGPDGGRRHHRRGSPEEAAEREPRRHRAHRHAPEPSREGAPGSKGERRARHRGGSRAGPREAESGEEPSRRHRARHKAPPAHEEAEKEAEAEDRDKEPRNHQPRESHCDLEASVVIGVGPVHALPSTCLEKVEEQPEDADNQRNVTRMGSQPSDLSTAVRIPVTLTGPPGETTVVPSGNVDLESQAEGKKEVEADDVMRSGPRPIVPYSSMFCLSPTNLLRRFCHYIVTMQYFEMVILVVIALSSIALAAEDPVQTDSPRNNVLKYMDYIFTGVFTFEMVIKMIDLGLLLHPGAYFRDLWNILDFIVVSGALVAFAFSGSKGKDISTIKSLRVLRVLRPLKTIKRLPKLKAVFDCVVNSLKNVLNILIVYMLFMFIFAVIAVQLFKGKFFYCTDESKELERDCRGQYLDYEKEEVEAQPRQWKKYDFHYDNVLWALLTLFTVSTGEGWPMVLKHSVDATYEEQGPSPGFRMELSIFYVVYFVVFPFFFVNIFVALIIITFQEQGDKVMSECSLEKNERACIDFAISAKPLTRYMPQNKQSFQYKTWTFVVSPPFEYFIMAMIALNTVVLMMKFYDAPYEYELMLKCLNIVFTSMFSMECVLKVIAFGVLNYFRDAWNVFDFVTVLGSITDILVTEIANNFINLSFLRLFRAARLIKLLRQGYTIRILLWTFVQSFKALPYVCLLIAMLFFIYAIIGMQVFGNIALDDDTSINRHNNFRTFLQALMLLFRSATGEAWHEIMLSCLSSRACDELANATECGSDFAYFYFVSFIFLCSFLMLNLFVAVIMDNFEYLTRDSSILGPHHLDEFIRVWAEYDPAACGRISYNDMFEMLKHMSPPLGLGKKCPARVAYKRLVRMNMPISNDDMTVHFTSTLMALIRTALEIKLAPAGTKQHQCDAELRKEISSVWANLPQKTLDLLVPPHKPDEMTVGKVYAALMIFDFYKQNKTTRDQIHQAPGGLAQMGPVSLFHPLKATLEQTQPAVLRGARVFLRQKSSASLSNGGAVQTQEGGIKESVSWGTQRTQEVPCEVRTPLERGHSTEIPVQTGKPAVDVQMQSMVLRGPDGEPQPGLESQGRAASMPRLAAETQPPPDASPMKRSISTLAPQRPHVAHLCTAALDRAPASQAAPPHHHRCHRRRDRKQKSLEKGPGLSADTDGAPYSTAGPGPPPPPGDGAAGCRRERRQERGRSQERRQPSSSSSEKQRFYSCDRFGGREPPQPKPSLSSHPTSPTAGQEPGPPRQGSGSVNGSPLLSTSGASTPGRGGRRQLPQTPLTPRPSITYKTANSSPVHFAGAQTSLPAFSPGRLSRGLSEHNALLQRDPLSQPLAPSSRIGSDPYLGQRLDSEAAARTQLEDTLTFEEAVATNSGRSSRTSYVSSLTSQSHPLRRVPNGYHCTLGLSSGGGGRGRHSYHHPDQDHWC

SEQ ID NO: 45 - protein, 33 aa, Homo sapiens
  MISC_FEATURE (1)..(33): Amino acid sequence encoded by exon 37a; amino acid at position 1 is partly encoded by exon 36, partly by exon 37a.  Identical amino acid sequence is found in Homo sapiens, Mus musculus and Rattus norvegicus
CCRIHYKDMYSLLRCIAPPVGLGKNCPRRLAYK

SEQ ID NO: 46 - protein, 32 aa, Rattus norvegicus
CCRIHYKDMYSLLRCIAPPVGLGKNCPRRLAY

SEQ ID NO: 47 - protein, 32 aa, Rattus norvegicus
CGRISYNDMFEMLKHMSPPLGLGKKCPARVAY